(19) United States
(12) Patent Application Publication
(10) Pub. No.: US 2004/0180916 A1
(43) Pub. Date: Sep. 16, 2004

(54) Treatment of pain with combinations of nalbuphine and other kappa-opioid receptor agonists and opioid receptor antagonists

(75) Inventor: Jon D. Levine, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA

(21) Appl. No.: 10/734,308

(22) Filed: Dec. 12, 2003

Related U.S. Application Data
(60) Provisional application No. 60/433,217, filed on Dec. 13, 2002.

(51) Int. Cl. A61K 31/485

(52) U.S. Cl. 514/282

(57) ABSTRACT

Methods and compositions for treating, managing or ameliorating pain in a subject (preferably, a mammal, and more preferably, a human) comprising administration of a centrally acting (i.e., crosses the blood brain barrier) agonist of a κ-opioid receptor and a centrally acting opioid antagonist such that the analgesia achieved by this administration is greater than with administration of either the κ-opioid receptor agonist or the opioid antagonist alone. Preferably the κ-opioid receptor is nalbuphine or a salt or prodrug of nalbuphine and the opioid antagonist is naloxone or a salt or prodrug of naloxone. Preferred methods of administration include mucosal (e.g. intranasal or pulmonary) and intravenous. Other κ-opioid receptors include pentazocine and butorphanol.

### CROSS-REFERENCES TO RELATED APPLICATIONS

This application claims the benefit of the priority of U.S. provisional application 60/433,217 filed Dec. 13, 2002, which is incorporated herein in its entirety.

### STATEMENT AS TO RIGHTS TO INVENTIONS MADE UNDER FEDERALLY SPONSORED RESEARCH OR DEVELOPMENT

The invention was made with government support under Grant Number NR 03923 of the National Institutes of Health. The government has certain rights in this invention.

### FIELD OF INVENTION

The present invention provides methods for treating, managing, and ameliorating pain, such as pain, including inflammatory pain, neuropathic pain, acute and chronic pain, and regional and generalized pain syndromes while avoiding adverse side effects such as abuse potential. In particular, the present invention provides methods of treating, managing, and ameliorating pain by administration of a generally low dose of a kappa opioid agonist with an opioid antagonist. The present invention also provides pharmaceutical compositions, pharmaceutical kits, and combination therapies for treating, managing, and ameliorating pain.

### BACKGROUND OF THE INVENTION

Opioids are a group of compounds that have opium or morphine-like properties. Opioids are primarily used to treat pain, but may have other pharmacological effects including drowsiness, respiratory depression, and constipation, as well as abuse potential and tolerance. An opioid agonist is a compound that binds to an opioid receptor and forms a complex which elicits pharmacological responses particular to the nature of the receptor. Kappa (κ)-opioid receptor agonists are compounds that induce analgesia predominantly by acting on kappa-opioid receptors. Examples of kappa-opioid agonists are nalbuphine, pentazocine, butorphanol, benzomorphan, and benzacetamide, phenothiazine, thiazine, and benzodiazepine derivatives.

Opioid antagonists are compounds that pharmacologically block or reverse all (or substantially all) the effects of opioid agonists. Non-selective opioid antagonists are those antagonists that act at least on κ, μ, and δ opioid receptors. Opioid antagonists are generally used to reverse the effects of opioid agonist overdose and treatment of opioid addiction. Examples of opioid antagonists include naloxone, naltrexone, methylnaltrexone and nalmefene.

To date, severe pain is treated with opioids, particularly μ opioids, such as morphine, which have significant adverse side effects, including abuse potential. What is needed is an analgesic that is sufficiently potent to treat severe and chronic pain with minimal adverse side effects. The present invention fills such a need since the combinations of κ-opioid receptor agonists and opioid antagonists have high analgesic potency with reduced adverse side effects compared to morphine and other μ opioid agonists.

### BRIEF SUMMARY OF INVENTION

The present invention provides methods of treating, managing or ameliorating pain (including, e.g., inflammatory pain, neuropathic pain, acute pain, chronic pain, generalized pain syndromes, post-operative and post-procedural pain, etc.) in a subject (preferably, a mammal, and more preferably, a human) comprising administration of a centrally acting (i.e., crosses the blood brain barrier) agonist of a κ-opioid receptor and a centrally acting opioid antagonist such that the analgesia achieved by this administration is greater than with administration of either the κ-opioid receptor agonist or the opioid antagonist alone (in certain circumstances, analgesia is achieved by administration of the combination whereas administration of the agonist or antagonist alone results in anti-analgesia or less analgesia than administration of the combination).

The invention further provides pharmaceutical compositions comprising combinations of a centrally acting κ-opioid agonist and an opioid antagonist that provide pain relief greater than the pain relief achieved with either the agonist or antagonist alone (and, preferably, greater than the additive analgesic effects of the agonist and antagonist on their own). The centrally acting κ-opioid agonist may be a benzomorphan derivative, such as, but not limited to, butorphanol, ketazocine, ethylketazocine, pentazocine, dezocine, and bremazocine; a benzacetamide derivative, such as, but not limited to U50,488, U69,593, U62,066 (spiradoline), PD 117302, CI-977, DuP 747, ICI 197067, ICI 199441, BRL 52537A, or BRL 52656A; a phenothiazine derivative, such as but not limited to Rp 60180; a thiazine derivative, such as but not limited to R-84760; or a benzodiazepine derivative, such as, but not limited to, Tifluadom. In certain doses, the agonist exhibits sexual dimorphism when administered alone, i.e., at certain doses induces more analgesia in women than men or causes anti-analgesia in men and analgesia in women in studies of groups comprising 15, 20, 30 or more subjects. In preferred embodiments, the κ-opioid receptor agonist is butorphanol or pentazocine, or, most preferably, nalbuphine. The opioid antagonist is also centrally-acting and is preferably naloxone, naltrexone, methylnaltrexone or nalmefene. The opioid antagonist is preferably a non-selective opioid antagonist, i.e., a compound that antagonizes at least κ, μ, and δ opioid receptors. The κ-opioid agonists and the opioid antagonists of the present invention may be in any pharmaceutically acceptable form, including the free base, a salt, a prodrug, or a mixture thereof.

In particular embodiments, the invention provides methods of intravenous and mucosal (e.g., nasal and pulmonary administration) administration of an amount of a centrally acting κ-opioid receptor agonist and an amount of an opioid antagonist that results in greater analgesia than results from administration of either the agonist or the antagonist alone. In one preferred embodiment, the invention provides methods of intravenous administration or mucosally administering a centrally acting κ-opioid receptor agonist with 0.02 mg to 8 mg, 0.02 to 7 mg, 0.02 mg to 6 mg, 0.02 to 5 mg, 0.02 to 4 mg, 0.02 to 3 mg, 0.02 to 2 mg, 0.02 to 2 mg, 0.1 mg to 8 mg, 0.1 mg to 7 mg, 0.1 mg to 6 mg, 0.1 mg to 5 mg, 0.1 mg to 4 mg, 0.1 mg to 3 mg, 0.1 mg to 2 mg, 0.1 mg to 1 mg, 0.2 mg to 8 mg, 0.2 mg to 7 mg, 0.2 mg to 6 mg, 0.2 mg to 4 mg, 0.2 mg to 3 mg, 0.2 mg to 2 mg, 0.2 mg to 1 mg, 0.4 mg to 8 mg, 0.4 to 7 mg, 0.4 to 6 mg, 0.4 mg to 5 mg, 0.4 mg to 4 mg, 0.4 mg to 3 mg, 0.4 mg to 2 mg, 0.4 mg to 1 mg, 0.5 mg to 8 mg, 0.5 mg to 6 mg, 0.5 mg to 5 mg, 0.5 mg to 4 mg, 0.5 mg to 3 mg, 0.5 mg to 3 mg, 0.5 to 2 mg, 0.5 mg to 1 mg, 1 mg to 8 mg, 1 mg to 6 mg, 1 mg to 5 mg, 1 mg to 4 mg, 1 mg to 3 mg, 1 mg to 2 mg, 5 mg to 8 mg, 4 mg to 7 mg, 3 mg to 5 mg, 0.02 mg to 1 mg, preferably 0.1 mg to 0.8 mg, 0.2 mg to 0.8 mg, 0.3 mg to 0.8 mg, 0.4 mg to 0.8 mg, 0.5 mg to 0.8 mg, 0.1 mg to 0.7 mg, 0.2 mg to 0.7 mg, 0.3 mg to 0.7 mg, 0.4 mg to 0.7 mg, 0.1 mg to 0.6 mg, 0.2 mg to 0.6 mg, 0.3 to 0.6 mg, 0.1 mg to 0.5 mg, 0.15 mg to 0.5 mg, 0.2 mg to 0.5 mg, 0.25 mg to 0.5 mg, 0.3 mg to 0.5 mg, 0.35 mg to 0.5 mg, 0.1 mg to 0.45 mg, 0.15 mg to 0.45 mg, 0.2 mg to 0.45 mg, 0.25 mg to 0.45 mg, 0.1 mg to 0.3 mg, 0.13 mg to 0.3 mg, 0.2 mg to 0.3 mg, 0.1 mg to 0.25 mg, 0.15 mg to 0.25 mg, 0.1 mg to 0.2 mg, 0.1 mg to 0.15 mg of a hydrochloride salt of the opioid antagonists described herein, and most preferably, of naloxone hydrochloride. Typical amounts of such hydrochloride salt are 0.1 mg, 0.15 mg, 0.2 mg, 0.25 mg, 0.3 mg, 0.35 mg, 0.4 mg, 0.45 mg, 0.5 mg, 0.55 mg, 0.6 mg 0.7 mg, 0.75 mg, 0.8 mg, 1 mg, 1.6 mg, 2 mg, 2.4 mg, 3 mg, 4 mg, 5 mg, 6 mg, 7 mg and 8 mg.

The invention also comprises administration of the amount of opioid antagonist (preferably naloxone) free base, a non-hydrochloride salt, a prodrug, or a mixture thereof that would result in the same blood concentration of the active naloxone or other opioid antagonist (or in certain embodiments, the active metabolite thereof) as would intravenous administration of the amount of the hydrochloride salt specified, i.e., contains an equivalent amount (i.e., the same number of moles) of the active opioid antagonist, or, in certain embodiments, such as sustained release formulations, results in release into the blood stream of an equivalent amount, but over a period of 15 minutes, 30 minutes, 1 hour, 2 hours, 4 hours, 6 hours, 8 hours, 10 hours, 12 hours, or 24 hours.

The present invention also encompasses methods comprising administration of opioid antagonist (preferably naloxone) free base, non-hydrochloride salt, prodrug, or mixture thereof in amounts equivalent to administration of the above amounts of the hydrochloride salt, i.e., an amount of naloxone or other opioid antagonist free base, non-hydrochloride salt, prodrug, or mixture thereof that produces the same blood concentration of the opioid antagonist (e.g., naloxone) or its active metabolite as the administration of a particular amount of hydrochloride salt. For example, approximately 0.1 mg naloxone hydrochloride salt is equivalent to approximately 0.09 mg naloxone free base, approximately 0.15 mg naloxone hydrochloride salt is approximately equivalent to 0.135 mg naloxone free base, approximately 0.2 mg of naloxone hydrochloride salt is equivalent to approximately 0.18 mg naloxone free base, and approximately 0.25 mg of naloxone hydrochloride salt is equivalent to approximately 0.225 mg naloxone free base.

In general, the amount of κ-opioid agonist to be administered with the antagonist is in the range of 5% to 100% of the recommended analgesic dose (e.g., as provided in the Physician's Desk Reference or other commonly used reference). Preferably, the dose of the κ-opioid agonist is 5% to 90%, 10% to 90%, 5% to 85%, 10% to 85%, 15% to 85%, 20% to 80%, 5% to 75%, 10% to 75%, 15% to 70%, 15.% to 60%, 5% to 55%, 10% to 55%, 15% to 50%, 5% to 50%, 5% to 45%, 5% to 40%, 5% to 35%, 5% to 30%, 10% to 40%, 10% to 35%, 10% to 30%, 15% to 40%, 15% to 35%, 15% to 30%, 5% to 25%, 10% to 25%, 15% to 25%, 5% to 20%, and 5% to 15% of the recommended analgesic dose.

In certain embodiments of the invention, the method comprises administration of nalbuphine, as the κ-opioid agonist, preferably by intravenous or intramucosal administration (e.g., by intranasal or pulmonary) administration, and more preferably such administration of nalbuphine hydrochloride salt. The nalbuphine hydrochloride salt is preferably administered in amounts 6.25 to 49 times greater, 6.25 to 40 times greater, 6.25 to 35 times greater, 6.25 to 30 times greater, 6.25 to 20 times greater, 8 to 35 times greater, 8 to 30 times greater, 8 to 25 times greater, 10 to 30 times greater, 10 to 20 times greater, 15 to 25 times greater, 20 to 25 times greater, 10 to 15 times greater, 9 to 15 times greater, 5 to 10 times greater, 30 to 49 times greater, 20 to 30 times greater, 15 to 20 times greater, 13 to 15 times greater, 11 to 13 times greater, 9 to 11 times greater, 7 to 9 times greater, or 6.25 to 7 times greater than the amount of opioid antagonist hydrochloride salt, preferably naloxone hydrochloride salt, administered.

Non-limiting examples of the present invention are methods comprising administration of 3 mg of nalbuphine hydrochloride salt with 0.4 mg naloxone hydrochloride salt (i.e., 7.5 times greater nalbuphine hydrochloride salt than the naloxone hydrochloride salt); 1.25 mg nalbuphine hydrochloride salt with 0.1 mg naloxone hydrochloride salt, 2.5 mg nalbuphine hydrochloride salt with 0.2 mg naloxone hydrochloride salt; 5 mg nalbuphine hydrochloride salt with 0.4 mg naloxone hydrochloride salt, 10 mg nalbuphine hydrochloride salt with 0.8 mg naloxone hydrochloride salt, 20 mg nalbuphine hydrochloride salt with 1.6 mg naloxone hydrochloride salt, 25 mg nalbuphine hydrochloride salt with 2.0 mg naloxone hydrochloride salt, and 30 mg nalbuphine hydrochloride salt with 2.4 mg naloxone hydrochloride salt (i.e., 12.5 times greater, by weight, nalbuphine hydrochloride salt than the naloxone hydrochloride salt); 5 mg nalbuphine hydrochloride salt with 0.2 mg naloxone hydrochloride salt and 10 mg nalbuphine hydrochloride salt with 0.4 mg naloxone hydrochloride salt (i.e., 25 times greater nalbuphine hydrochloride salt than the naloxone hydrochloride salt); 4.9 mg nalbuphine hydrochloride salt with 0.1 mg naloxone hydrochloride salt and 9.8 mg nalbuphine hydrochloride salt with 0.2 mg naloxone hydrochloride salt (i.e., 49 times greater, by weight, nalbuphine hydrochloride salt than naloxone hydrochloride salt). Alternatively, 1 mg to 50 mg, 1 mg to 45 mg, 1 mg to 40 mg, 1 mg to 35 mg, 1 mg to 30 mg, 1 mg to 25 mg, 1 mg to 20 mg, 1 mg to 15 mg, 1 mg to 10 mg, 1 mg to 9 mg, 1 mg to 8 mg, 2 mg to 8 mg, 3 mg to 8 mg, 1 mg to 7 mg, 2 mg to 7 mg, 3 mg to 7 mg, 4 mg to 7 mg, 1 mg to 6 mg, 2 mg to 6 mg, 3 mg to 6 mg, 1 mg to 5 mg, 2 mg to 5 mg, 1 mg to 4 mg, 1 mg to 3 mg, 1 mg to 2 mg nalbuphine hydrochloride salt is administered.

In certain preferred embodiments in which nalbuphine hydrochloride salt is administered as the opioid agonist, 0.02 mg to 8 mg, preferably, 0.1 mg to 0.8 mg naloxone hydrochloride salt is administered as the opioid antagonist hydrochloride salt. Non-limiting examples of the method of the present invention comprises 1 mg, 1.25 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 15 mg, 20 mg, 25 mg, 30 mg, 40 mg, or 50 mg of nalbuphine hydrochloride salt with 0.1 mg, 0.15 mg, 0.2 mg, 0.2 mg, 0.25 mg, 0.3 mg, 0.35 mg, 0.4 mg, 0.5 mg, 0.6 mg, 0.7 mg, 0.8 mg, 1 mg, 1.6 mg, 2.0 mg, 2.4 mg, 3.0 mg, 4 mg, 5 mg, 6 mg, 7 mg, or 8 mg of naloxone hydrochloride salt.

For example, specific embodiments of the invention comprise of intravenous or mucosal (e.g., nasal or pulmonary) administration of 0.2 mg of naloxone hydrochloride salt with 1 mg nalbuphine hydrochloride salt, 0.1 mg naloxone hydrochloride salt with 1.25 mg nalbuphine hydrochloride salt, 0.2 mg of naloxone hydrochloride salt with 1.5 mg nalbuphine hydrochloride salt, 0.2 mg of naloxone hydrochloride salt with 2 mg nalbuphine hydrochloride salt, 0.2 mg of naloxone hydrochloride salt with 2.5 mg nalbuphine hydrochloride salt, 0.2 mg of naloxone hydrochloride salt with 3 mg nalbuphine hydrochloride salt, 0.2 mg of naloxone hydrochloride salt with 3.5 mg nalbuphine hydrochloride salt, 0.2 mg of naloxone hydrochloride salt with 4 mg nalbuphine hydrochloride salt, 0.2 mg of naloxone hydrochloride salt with 4.5 mg nalbuphine hydrochloride salt, 0.2 mg of naloxone hydrochloride salt with 5 mg nalbuphine hydrochloride salt, 0.2 mg of naloxone hydrochloride salt with 5.5 mg nalbuphine hydrochloride salt, 0.2 mg of naloxone hydrochloride salt with 6 mg nalbuphine hydrochloride salt, 0.2 mg of naloxone hydrochloride salt with 6.5 mg nalbuphine hydrochloride salt, 0.2 mg of naloxone hydrochloride salt with 7 mg nalbuphine hydrochloride salt 0.2 mg of naloxone hydrochloride salt with 7.5 mg nalbuphine hydrochloride salt, 0.2 mg of naloxone hydrochloride salt with 8 mg nalbuphine hydrochloride salt, 0.2 mg of naloxone hydrochloride salt with 8.5 mg nalbuphine hydrochloride salt, 0.2 mg of naloxone hydrochloride salt with 9 mg nalbuphine hydrochloride salt, 0.2 mg of naloxone hydrochloride salt with 9.5 mg nalbuphine hydrochloride salt, 0.2 mg of naloxone hydrochloride salt with 10 mg nalbuphine hydrochloride salt, 0.4 mg of naloxone hydrochloride salt with 1 mg nalbuphine hydrochloride salt, 0.4 mg of naloxone hydrochloride salt with 1.5 mg nalbuphine hydrochloride salt, 0.4 mg of naloxone hydrochloride salt with 2.0 mg nalbuphine hydrochloride salt, 0.4 mg of naloxone hydrochloride salt with 2.5 mg nalbuphine hydrochloride salt, 0.4 mg of naloxone hydrochloride salt with 3 mg nalbuphine hydrochloride salt, 0.4 mg of naloxone hydrochloride salt with 3.5 mg nalbuphine hydrochloride salt, 0.4 mg of naloxone hydrochloride salt with 4 mg nalbuphine hydrochloride salt, 0.4 mg of naloxone hydrochloride salt with 4.5 mg nalbuphine hydrochloride salt, 0.4 mg of naloxone hydrochloride salt with 5 mg nalbuphine hydrochloride salt, 0.4 mg of naloxone hydrochloride salt with 5.5 mg nalbuphine hydrochloride salt, 0.4 mg of naloxone hydrochloride salt with 6 mg nalbuphine hydrochloride salt, 0.4 mg of naloxone hydrochloride salt with 6.5 mg nalbuphine hydrochloride salt, 0.4 mg of naloxone hydrochloride salt with 7 mg nalbuphine hydrochloride salt, 0.4 mg of naloxone hydrochloride salt with 7.5 mg nalbuphine hydrochloride salt, 0.4 mg of naloxone hydrochloride salt with 8 mg nalbuphine hydrochloride salt, 0.4 mg of naloxone hydrochloride salt with 8.5 mg nalbuphine hydrochloride salt, 0.4 mg of naloxone hydrochloride salt with 9 mg nalbuphine hydrochloride salt, 0.4 mg of naloxone hydrochloride salt with 9.5 mg nalbuphine hydrochloride salt, 0.4 mg of naloxone hydrochloride salt with 10 mg nalbuphine hydrochloride salt, and 0.8 mg naloxone hydrochloride salt with 10 mg nalbuphine hydrochloride salt.

The present invention also encompasses methods comprising intravenous or mucosal administration of nalbuphine free base, non-hydrochloride salt, prodrug, or a mixture thereof in amounts equivalent to the specified amounts of nalbuphine hydrochloride salt, provided that 5 mg of nalbuphine free base is not administered with 0.4 mg of naloxone free base intravenously. As stated above with regard to naloxone, an equivalent amount of nalbuphine free base, non-hydrochloride salt, prodrug, or mixture thereof is an amount that produces the same blood concentration of nalbuphine or active metabolite of nalbuphine as would intravenous administration of the specified amount of nalbuphine hydrochloride salt or the same amount of nalbuphine released into the blood stream over a period of 15 minutes, 30 minutes, 1 hour, 2 hours, 4 hours, 6 hours, 8 hours, 10 hours, 12 hours, or 24 hours. For example, approximately 1 mg of nalbuphine hydrochloride salt administered intravenously is equivalent to approximately 0.9 mg of nalbuphine free base administered intravenously.

In certain embodiments, the method of the invention comprises intravenous administration of pentazocine as the κ-opioid agonist, preferably as pentazocine hydrochloride salt. In particular, the invention provides methods of administering intravenously an opioid antagonist hydrochloride salt, preferably naloxone hydrochloride salt, and 18 to 120 times greater, 25 to 120 times greater, 18 to 110 times greater, 25 to 110 times greater. 18 to 100 times greater, 25 to 100 times greater, 18 to 95 times greater, 25 to 90 times greater, 30 to 90 times greater, 18 to 85 times greater, 18 to 80 times greater, 20 to 80 times greater, 20 to 60 times greater, 20 to 50 times greater, 25 to 55 times greater, 35 to 80 times greater, 20 to 75 times greater, 25 to 70 times greater, 40 to 100 times greater, 50 to 100 times greater, 55 to 95 times greater, 45 to 90 times greater, 40 to 70 times greater, 18 to 50 times greater, 18 to 40 times greater, or 18 to 35 times greater, 18 to 30 times greater, by weight, pentazocine hydrochloride salt. Non-limiting examples of the methods of the present invention comprise intravenous administration of 10 mg of pentazocine hydrochloride salt with 0.4 mg of naloxone hydrochloride salt (i.e., 25 times greater, by weight, pentazocine hydrochloride salt than the naloxone hydrochloride salt), 15 mg of pentazocine hydrochloride salt with 0.3 mg naloxone hydrochloride salt (i.e., 30 times greater, by weight, pentazocine hydrochloride salt than the naloxone hydrochloride salt), and 25 mg of pentazocine hydrochloride salt with 0.5 mg naloxone hydrochloride salt (i.e., 50 times greater pentazocine hydrochloride salt than naloxone hydrochloride salt). Preferably, the weight of pentazocine hydrochloride salt administered is 3 mg to 50 mg, 4 mg to 50 mg, 5 mg to 50 mg, 6 mg to 50 mg, 7 mg to 50 mg, 3 mg to 45 mg, 5 mg to 45 mg, 10 mg to 45 mg, 15 mg to 45 mg, 5 mg to 40 mg, 10 mg to 40 mg, 3 mg to 35 mg, 4 mg to 35 mg, 5 mg to 35 mg, 10 mg to 35 mg, 3 mg to 30 mg, 4 mg to 30 mg, 5 mg to 30 mg, 3 mg to 25 mg, 4 mg to 25 mg, 3 mg to 20 mg, 4 mg to 20 mg, 5 mg to 25 mg, 10 mg to 25 mg, 15 mg to 30 mg, 15 mg to 25 mg, 10 mg to 20 mg, and 10 mg to 15 mg.

In certain embodiments in which pentazocine hydrochloride salt is administered, 0.02 mg to 8 mg, preferably 0.1 mg to 0.8 mg of naloxone hydrochloride is preferably administered as the opioid antagonist hydrochloride salt. Non-limiting examples of the present invention are methods comprising administration of 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 30 mg, 35 mg, 40 mg, 45 mg, and 50 mg pentazocine hydrochloride salt with 0.1 mg, 0.15 mg, 0.2 mg, 0.2 mg, 0.25 mg, 0.3 mg, 0.35 mg, 0.4 mg, 0.8 mg, 1 mg, 2 mg, 3 mg, 4 mg, 5 mg, 6 mg, 7 mg, or 8 mg naloxone hydrochloride salt.

Additionally, in certain embodiments, the invention comprises administration equivalent amounts of pentazocine free base, non-hydrochloride salt, prodrug, or mixture thereof to the specified amounts of pentazocine hydrochloride salt, i.e., contains the same amount (i.e., same number of moles) of active pentazocine and/or results in the same blood concentration of pentazocine or active metabolite of pentazocine as a particular amount of pentazocine hydrochloride salt, or, in certain embodiments, such as sustained release formulations, results in release into the blood stream of an equivalent amount, but over a period of 15 minutes, 30 minutes, 1 hour, 2 hours, 4 hours, 6 hours, 8 hours, 10 hours, 12 hours, or 24 hours.

In other embodiments, the method of the invention comprises intravenous administration of butorphanol, e.g. as the tartrate salt, as the κ-opioid agonist. The butorphanol tartrate salt form is preferably administered intravenously at 0.3 to 10 times greater, 0.3 to 9 times greater, 0.3 to 8 times greater, 0.5 to 10 times greater, 0.3 to 7 times greater, 0.5 to 7 times greater, 0.3 to 6 times greater. 0.5 to 6 times greater, 0.3 to 5 times greater, 0.5 to 5 times greater, 0.3 to 4 times greater, 0.5 to 4 times greater, 0.3 to 3 times greater, or 0.5 to 3 times greater, by weight, than the amount of opioid antagonist hydrochloride salt, preferably naloxone hydrochloride salt, administered. Non-limiting examples of the present invention are methods comprising administration of 0.3 mg of butorphanol tartrate salt with 0.3 mg of naloxone hydrochloride salt (i.e., 1 time greater, by weight, of butorphanol tartrate salt than naloxone hydrochloride salt), 0.5 mg of butorphanol tartrate salt with 0.2 mg naloxone hydrochloride salt (i.e., 2.5 times greater, by weight, butorphanol tartrate salt than naloxone hydrochloride salt), and 0.8 mg of butorphanol tartrate salt with 0.4 mg naloxone hydrochloride salt (i.e., 2 times greater, by weight butorphanol tartrate salt than naloxone hydrochloride salt). Alternatively, the weight of butorphanol tartrate salt administered is 0.2 mg to 2 mg, 0.2 mg to 1.9 mg, 0.2 mg to 1.8 mg, 0.2 mg to 1.7 mg, 0.25 mg to 1.9 mg, 0.25 mg to 1.8 mg, 0.25 mg to 1.75 mg, 0.25 mg to 1.5 mg, 0.25 mg to 1 mg, or 0.2 mg to 1 mg.

In certain preferred embodiments comprising administration of butorphanol tartrate salt, 0.02 mg to 8 mg, preferably 0.1 mg to 0.8 mg of naloxone hydrochloride salt is administered as the opioid antagonist hydrochloride. Non-limiting examples of the present invention are methods comprising administration of 0.2 mg, 0.3 mg, 0.4 mg, 0.5 mg, 0.6 mg, 0.7 mg, 0.8 mg, 0.9 mg, 1.0 mg, 1.1 mg, 1.1 mg, 1.2 mg, 1.3 mg, 1.4 mg, 1.5 mg, 1.6 mg, 1.7 mg, 1.8 mg, or 2.0 mg of butorphanol tartrate salt with 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.7 mg, 0.75 mg, 0.8 mg, 1 mg, 2 mg, 3 mg, 4 mg, 5 mg, 6 mg, 7 mg, or 8 mg naloxone hydrochloride salt.

Additionally, the invention comprises administration of equivalent amounts of butorphanol free base, non-tartrate salt, prodrug, or mixture to the specified amounts of butorphanol tartrate salt, i.e., contains an equivalent amount (i.e., the same number of moles) of the active butorphanol, or, in certain embodiments, such as sustained release formulations, results in release into the blood stream of an equivalent amount, but over a period of 15 minutes, 30 minutes, 1 hour, 2 hours, 4 hours, 6 hours, 8 hours, 10 hours, 12 hours, or 24 hours.

The invention further provides methods of treating, ameliorating or managing pain by administering the κ-opioid agonist/opioid antagonist combinations of the invention by modes of administration other than intravenous administration and, in certain embodiments, other than oral administration for gastro-intestinal uptake, preferably, by mucosal administration, such as, but not limited to, sublingual, intranasal, inhalation (e.g., pulmonary), suppository (e.g., rectal) but also by buccal, intramuscular, subcutaneous or other parenteral administration, or, in certain cases, by oral, transdermal (e.g., patch), etc. These methods comprise administering, by a method other than intravenous administration, amounts of the κ-opioid agonist free base, salt, prodrug, or mixture thereof and the opioid antagonist free base, salt, prodrug, or mixture thereof that result in the same blood concentration of the κ-opioid agonist or the active metabolite of the κ-opioid agonist and opioid antagonist or active metabolite of opioid antagonist as would result from intravenous administration of the amounts of the κ-opioid agonist and opioid antagonists discussed above for intravenous administration. The exact amounts of the κ-opioid agonist and opioid antagonist will vary depending on the method of administration chosen due to different absorption rates and bioavailability of the agonist and antagonist.

In certain preferred embodiments of the invention, the method of administration is sublingual (or other oral cavity administration). In embodiments comprising sublingual administration of nalbuphine, the amount of nalbuphine hydrochloride salt is 1 to 60 times greater, 1 to 50 times greater, 1 to 45 times greater, 1 to 40 times greater, 5 to 50 times greater, 5 to 40 times greater, 5 to 35 times greater, 10 to 40 times greater, 15 to 40 times greater, 10 to 30 times greater, 15 to 30 times greater, 1 to 30 times greater, 1 to 20 times greater, 1 to 15 times greater, or 1 to 9 times greater, by weight, than the amount of opioid antagonist hydrochloride salt, preferably naloxone hydrochloride salt, administered. Non-limiting examples of methods of the present invention comprise sublingual administration of 8 mg nalbuphine hydrochloride salt with 0.4 mg naloxone hydrochloride salt (i.e., 20 times greater, by weight, nalbuphine hydrochloride salt than the opioid antagonist hydrochloride salt), 15 mg nalbuphine hydrochloride salt with 3 mg naloxone hydrochloride salt (i.e., 5 times greater, by weight, nalbuphine hydrochloride salt than the opioid antagonist hydrochloride salt), and 30 mg nalbuphine hydrochloride salt with 4 mg naloxone hydrochloride salt (i.e., 7.5 times greater, by weight, nalbuphine hydrochloride salt than the opioid antagonist hydrochloride salt). Alternatively, 5 mg to 65 mg, 5 mg to 60 mg, 5 mg to 55 mg, 5 mg to 50 mg, 5 mg to 40 mg, 5 mg to 35 mg, 6 mg to 55 mg, 6 mg to 50 mg, 6 mg to 45 mg, 6 mg to 40 mg, 6 mg to 30 mg, 7 mg to 40 mg, 7 mg to 35 mg, or 7.5 to 30 mg of nalbuphine hydrochloride salt is administered sublingually with an amount of naloxone that enhances nalbuphine analgesia.

In certain preferred embodiments of the invention in which the method comprises sublingual administration of nalbuphine hydrochloride salt, naloxone hydrochloride salt is preferably administered sublingually as the opioid antagonist hydrochloride in amounts preferably 0.1 mg to 10 mg, 0.1 mg to 9 mg, 0.1 mg to 8 mg, 0.1 mg to 7 mg, 0.1 mg to 6 mg, 0.2 mg to 10 mg, 0.2 mg to 9 mg, 0.2 mg to 8 mg, 0.2 mg to 7 mg, 0.2 mg to 6 mg, 0.2 mg to 5 mg, 0.3 mg to 10 mg, 0.3 mg to 9 mg, 0.3 mg to 7 mg, 0.3 mg to 6 mg, 0.3 mg to 5 mg, 0.3 mg to 4 mg, 0.4 mg to 4 mg, 0.5 mg to 4 mg, 0.4 mg to 3.5 mg, 0.5 mg to 3.5 mg, 0.6 mg to 3.5 mg, 0.4 mg to 3 mg, 0.5 mg to 3 mg, 0.4 mg to 2.8 mg, 0.6 mg to 2.7 mg, 0.4 mg to 2.5 mg, 0.5 mg to 2.2 mg, 0.4 mg to 2 mg, 0.6 mg to 2 mg, 0.4 mg to 1.5 mg, or 0.4 mg to 1 mg. Non-limiting examples of the present invention are methods comprising administration of 5 mg, 5.5 mg, 6 mg, 6.5 mg, 7 mg, 7.5 mg, 8 mg, 8.5 mg, 9 mg, 9.5 mg 10 mg, 15 mg, 20 mg, 30 mg, 35 mg, 40 mg, 45 mg or 50 mg nalbuphine hydrochloride salt with naloxone hydrochloride salt.

For example, in specific embodiments of the present invention, the method comprises sublingual administration of 0.4 mg of naloxone hydrochloride salt with 5 mg of nalbuphine hydrochloride salt, 0.4 mg of naloxone hydrochloride salt with 6 mg of nalbuphine hydrochloride salt, 0.4 mg of naloxone hydrochloride salt with 7 mg of nalbuphine hydrochloride salt, 0.4 mg of naloxone hydrochloride salt with 8 mg of nalbuphine hydrochloride salt, 0.4 mg of naloxone hydrochloride salt with 9 mg of nalbuphine hydrochloride salt, 0.4 mg of naloxone hydrochloride salt with 10 mg of nalbuphine hydrochloride salt, 0.4 mg of naloxone hydrochloride salt with 15 mg of nalbuphine hydrochloride salt, 0.4 mg of naloxone hydrochloride salt with 20 mg of nalbuphine hydrochloride salt, 0.4 mg of naloxone hydrochloride salt with 25 mg of nalbuphine hydrochloride salt, and 0.4 mg of naloxone hydrochloride salt with 30 mg of nalbuphine hydrochloride salt.

As discussed above, the invention also encompasses sublingual administration of amounts of nalbuphine and/or naloxone free base, non-hydrochloride salt, prodrug, or mixtures thereof equivalent to the amounts of nalbuphine hydrochloride salt and naloxone hydrochloride salt recited immediately above. In other embodiments, the invention provides, for example, controlled or sustained release formulations that release equivalent amounts of nalbuphine and naloxone to the site of administration (e.g., the oral cavity) over a period of 15 minutes, 30 minutes, 1 hour, 2 hours, 4 hours, 6 hours, 8 hours, 10 hours, 12 hours, or 24 hours.

In certain embodiments comprising sublingual administration of pentazocine, the amount of pentazocine hydrochloride salt is 7.5 to 50 times greater, 7.5 to 45 times greater, 10 to 50 times greater, 10 to 40 times greater, 15 to 50 times greater, 15 to 45 times greater, 7.5 to 30 times greater, 7.5 to 25 times greater, 7.5 to 20 times greater, 10 to 30 times greater, or 7.5 to 15 times greater, by weight, than the amount of opioid antagonist hydrochloride salt administered. Alternatively, the amount of pentazocine hydrochloride salt administered sublingually is 30 mg to 100 mg, 30 to 90 mg, 40 mg to 100 mg, 30 mg to 85 mg, 40 mg to 85 mg, 30 mg to 70 mg, 40 mg to 70 mg, 30 mg to 60 mg, 40 mg to 60 mg, 30 mg to 50 mg, 50 mg to 80, or 30 mg to 45 mg.

In embodiments of the invention in which pentazocine hydrochloride salt is administered sublingually, naloxone hydrochloride salt is the preferred opioid antagonist hydrochloride salt and is administered in amounts 1 mg to 10 mg, 1 mg to 9 mg, 1 mg to 8 mg, 1 mg to 7 mg, 1 mg to 6 mg, 1 mg to 5 mg, 1 mg to 4 mg, 2 mg to 10 mg, 2 mg to 9 mg, 2 mg to 8 mg, 2 mg to 7 mg, 2 mg to 6 mg, 2 mg to 5 mg, 2 mg to 4 mg, 2 mg to 3.8 mg, 2 mg to 3.6 mg, 2 mg to 3.4 mg, 2 mg to 3.4 mg, 2.2 mg to 3.5 mg, 2 mg to 3.2 mg, 3 mg to 4 mg, or 2 mg to 3 mg. Non-limiting examples of the present invention are methods comprising sublingual administration of 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 50 mg, 55 mg, 60 mg, 65 mg, 70 mg, 75 mg, 80 mg, 85 mg, 90 mg, 95 mg or 100 mg pentazocine hydrochloride salt with 2 mg, 2.1 mg, 2.2 mg, 2.3 mg, 2.4 mg, 2.5 mg, 2.6 mg, 2.7 mg, 2.8 mg, 2.9 g, 3 mg, 3.1 mg, 3.2 mg, 3.3 mg, 3.4 mg, 3.5 mg, 3.6 mg, 3.7 mg, 3.8 mg, 3.9 mg, or 4.0 mg naloxone hydrochloride salt.

As discussed above, the invention also encompasses sublingual administration of amounts of pentazocine and/or naloxone free base, non-hydrochloride salt, prodrug, or mixtures thereof equivalent to the amounts of pentazocine hydrochloride salt and naloxone hydrochloride salt recited immediately above. In other embodiments, the invention provides, for example, controlled or sustained release formulations that release equivalent amounts of pentazocine and naloxone to the site of administration (e.g., the oral cavity) over a period of 15 minutes, 30 minutes, 1 hour, 2 hours, 4 hours, 6 hours, 8 hours, 10 hours, 12 hours, or 24 hours.

In certain embodiments comprising sublingual administration of butorphanol as the κ-opioid agonist, butorphanol tartrate salt is administered in amounts 0.1 to 60 times greater, 0.1 to 50 times greater, 0.1 to 45 times greater, 0.3 to 40 times greater, or 0.5 to 30 greater, 10 to 60 times greater, 20-60 times greater, and 10-30 times greater, by weight, than the opioid antagonist hydrochloride salt, preferably naloxone hydrochloride salt, administered. Non-limiting examples of the present invention are methods comprising sublingual administration of 0.5 mg butorphanol tartrate salt with 0.25 mg naloxone hydrochloride salt (i.e., 2 times greater, by weight, butorphanol tartrate salt than the opioid antagonist hydrochloride salt), 2 mg butorphanol tartrate salt with 0.2 mg naloxone hydrochloride salt (i.e., 10 times greater, by weight, butorphanol tartrate salt than the opioid antagonist hydrochloride salt), and 6 mg butorphanol tartrate salt with 0.3 mg naloxone hydrochloride salt (i.e., 20 times greater, by weight, butorphanol tartrate salt than the opioid antagonist hydrochloride salt). Alternatively, 0.1 mg to 10 mg, 0.1 mg to 9 mg, 0.1 mg to 8 mg, 0.1 mg to 7 mg, 0.1 mg to 6 mg, 0.2 mg to 9 mg, 0.2 mg to 8 mg, 0.2 mg to 7 mg, 0.2 mg to 6 mg, 0.2 mg to 5.5 mg, 0.3 to 6.5 mg, 0.4 mg to 7 mg, or 0.5 mg to 6 mg of butorphanol tartrate salt is administered sublingually.

If the κ-opioid agonist is butorphanol tartrate salt, then, preferably, 0.1 to 4 mg, 0.1 mg to 3.5 mg, 0.1 mg to 3 mg, 0.1 to 2.5 mg, 0.1 mg to 2 mg, 0.1 mg to 1 mg, 0.3 mg to 0.8 mg, or 0.1 mg to 0.8 mg of naloxone hydrochloride salt is administered. Non-limiting examples of the present invention are methods comprising administration of 0.1 mg, 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, or 7 mg butorphanol tartrate salt with 0.1 mg, 0.2 mg, 0.3 mg, 0.4 mg, 0.5 mg, 0.6 mg, 0.7 mg, or 0.85 mg naloxone hydrochloride salt.

As discussed above, the invention also encompasses sublingual administration of equivalent amounts of butorphanol and/or naloxone free base, other salt, prodrug, or mixtures thereof to the amounts of butorphanol tartrate salt and naloxone hydrochloride salt recited immediately above. In other embodiments, the invention provides, for example, controlled or sustained release formulations that release equivalent amounts of butorphanol and naloxone to the site of administration (e.g., the oral cavity) over a period of 15 minutes, 30 minutes, 1 hour, 2 hours, 4 hours, 6 hours, 8 hours, 10 hours, 12 hours, or 24 hours.

In a preferred form of the invention, the combination of the κ-opioid agonist, preferably nalbuphine, for example as the hydrochloride salt, and opioid antagonist, preferably naloxone, for example as the hydrochloride salt, is administered to the patient or subject (preferably a human) by mucosal administration, particularly by intranasal or pulmonary administration. In such administration a composition containing the two ingredients suitable for such administration, is used, as described hereinafter. In such administration, the amounts of the opioid agonist and antagonist, and the ratio of the one to the other, are as described above. Particularly good results are obtained by administration of the combination wherein the weight ratio of nalbuphine component to naloxone component is from about 10:1 to about 15:1, most preferably about 12.5:1 and where the amount of nalbuphine (as the hydrochloride salt) is 5 mg and the amount of naloxone (as the hydrochloride salt) is about 0.4 mg.

The two ingredients also may be administered at one-quarter of the appropriate dosage (e.g., 1.25 nalbuphine hydrochloride and 0.1 mg naloxone hydrochloride), at one-half of that dosage (e.g., 2.5 mg nalbuphine hydrochloride and 0.2 mg naloxone hydrochloride) or at twice (e.g., 10 mg nalbuphine hydrochloride and 0.8 mg naloxone hydrochloride) or four times that dosage (e.g., 20 mg nalbuphine hydrochloride and 1.6 mg naloxone hydrochloride). Such dosage variations may be used, for example such that patients of less body mass or less pain would receive higher doses and patients with greater body mass or more severe pain would receive higher doses.

More specifically, in one embodiment the invention comprises a method of treating pain comprising mucosally administering to an individual in need of said treatment (a) 0.02 mg to 8 mg, preferably 0.1 mg to 0.8 mg of a hydrochloride salt of an opioid antagonist or an equivalent amount of opioid antagonist free base, prodrug, non-hydrochloride salt, or mixture thereof, wherein said opioid antagonist is naloxone, naltrexone, methylnaltrexone, nalmefene, nalorphine, levalorphan, oxylorphan or cyprenorphine, and (b) an amount of nalbuphine free base, hydrochloride salt, prodrug, non-hydrochloride salt or mixture thereof that results in greater analgesia than administration of either said opioid antagonist hydrochloride salt or said nalbuphine alone. The invention further comprises compositions comprising such ingredients in such amounts.

In another embodiment, the invention comprises a method of treating pain comprising administering to an individual in need of said treatment (a) 0.02 mg to 8 mg, preferably 0.1 mg to 0.8 mg of a hydrochloride salt of an opioid antagonist or an equivalent amount of opioid antagonist free base, prodrug, non-hydrochloride salt, or mixture thereof, wherein said opioid antagonist is naloxone, naltrexone, methylnaltrexone, nalmefene, nalorphine, levalorphan, oxylorphan or cyprenorphine, and (b) an amount of a free base, hydrochloride salt, prodrug, non-hydrochloride salt or mixture thereof of a kappa opioid, that results in greater analgesia than administration of either said opioid antagonist hydrochloride salt or said kappa-opioid, wherein the kappa-opioid is pentazocine, butorphanol, ketazocine, ethylketazocine, dezocine, bremazocine, a benzacetamide derivative, a phenothiazine derivative, a thiazine derivative, or a benzodiazepine derivative, provided that 60 mg of pentazocine free base or an equivalent amount of pentazocine hydrochloride salt, non-hydrochloride salt, prodrug, or mixture thereof, is not administered with 0.4 mg naloxone free base or equivalent amount of naloxone hydrochloride salt, non-hydrochloride salt, prodrug, or mixture thereof. Here, too the invention also comprises such compositions.

In yet another embodiment the invention comprises a method of treating pain comprising administering to an individual in need of said treatment (a) from about 0.1 to about 0.8 mg of a hydrochloride salt of naloxone or an equivalent amount of naloxone free base, prodrug, non-hydrochloride salt, or mixture thereof; and either (b) from about 1 to about 2.5 mg, or (c) from about 8.5 to about 10.0 mg, of nalbuphine hydrochloride, or an equivalent amount of nalbuphine free base, prodrug, non-hydrochloride salt or mixture thereof. Here, too, the invention further comprises compositions for use in both methods.

In still another embodiment the invention comprises a method of treating pain comprising administering to an individual in need of said treatment (a) 0.2 mg of a hydrochloride salt of naloxone or an equivalent amount of naloxone free base, prodrug, non-hydrochloride salt, or mixture thereof; and (b) 2.5 mg of nalbuphine hydrochloride, or an equivalent amount of nalbuphine free base, prodrug, non-hydrochloride salt or mixture thereof. The invention also comprises a method of treating pain comprising administering to an individual in need of said treatment (a) 0.8 mg of a hydrochloride salt of naloxone or an equivalent amount of naloxone free base, prodrug, non-hydrochloride salt, or mixture thereof; and (b) 10 mg of nalbuphine hydrochloride, or an equivalent amount of nalbuphine free base, prodrug, non-hydrochloride salt or mixture thereof. Here, too, the invention further comprises compositions for use in both methods.

The invention also provides pharmaceutical compositions, formulations, and dosage units comprising the κ-opioid agonist-opioid antagonist combinations of the invention.

### BRIEF DESCRIPTION OF DRAWINGS

FIGS. 1a-d depicts the effects on postoperative pain of administration of 2.5 mg, 5 mg, and 10 mg nalbuphine hydrochloride salt and 0.4 mg naloxone hydrochloride salt alone and in combination with each other in 264 subjects who had undergone surgery for removal of third molar (“wisdom”) teeth.

FIGS. 2a-d depicts the effects on postoperative pain of administration of 2.5 mg, 5 mg, and 10 mg nalbuphine hydrochloride salt and 0.4 mg naloxone hydrochloride salt alone and in combination with each other in 281 patients who had undergone surgery for removal of third molar (“wisdom”) teeth.

FIGS. 3a-b depicts the effects on postoperative pain of administration of 5 mg nalbuphine hydrochloride salt with 0.1 mg, 0.2 mg, and 0.4 mg naloxone hydrochloride salt in 52 patients who had undergone surgery for removal of third molar (“wisdom”) teeth.

FIGS. 4a-b shows the effects on postoperative pain of administration of 2.5 mg nalbuphine hydrochloride salt and 2.5 mg nalbuphine hydrochloride salt with 0.4 mg naloxone hydrochloride salt in 67 patients who had undergone surgery for removal of third molar (“wisdom”) teeth.

FIGS. 5a-b shows the effects on postoperative pain of administration of 2.5 mg of nalbuphine hydrochloride alone and in combination with 0.2 mg naloxone hydrochloride to 65 patients who had undergone oral surgery for removal of third molar (“wisdom”) teeth.

FIG. 6 shows visual analog scale (VAS) pain scores for three patients who received repeated intravenous administrations of 5 mg nalbuphine hydrochloride plus 0.4 mg naloxone hydrochloride to treat postoperative pain following Le Fort I osteotomy.

FIG. 7 shows visual analog scale (VAS) pain scores for three patients who received repeated intravenous administrations of 5 mg nalbuphine hydrochloride plus 0.4 mg naloxone hydrochloride to treat postoperative pain following Le Fort I osteotomy.

FIG. 8 shows visual analog scale (VAS) pain scores for three patients who received repeated intravenous administrations of 5 mg nalbuphine hydrochloride plus 0.4 mg naloxone hydrochloride to treat postoperative pain following Le Fort I osteotomy.

FIG. 9 shows visual analog scale (VAS) pain scores for three patients who received 5 mg nalbuphine hydrochloride with 0.4 mg naloxone hydrochloride by intravenous catheter for treatment of medically refractory trigeminal neuropathy.

FIG. 10 shows the relative change in pain for three patients who received 5 mg nalbuphine hydrochloride with 0.4 mg naloxone hydrochloride by intravenous catheter for treatment of medically refractory trigeminal neuropathy.

FIG. 11 shows the effects on postoperative pain of intranasal administration of 5 mg of nalbuphine hydrochloride alone and in combination with 0.4 mg naloxone hydrochloride to 5 patients who had undergone oral surgery for removal of third molar (“wisdom”) teeth. This figure includes a comparison with intravenous administration of the combination of 5 mg nalbuphine hydrochloride and 0.4 mg naloxone hydrochloride.

### DETAILED DESCRIPTION OF THE INVENTION

According to the present invention, greater analgesia is achieved by administration of a centrally acting κ-opioid receptor agonist with a centrally acting opioid antagonist than administration of either the κ-opioid receptor agonist or the opioid antagonist alone (and, in certain embodiments, greater than the additive analgesic effect of the agonist and antagonist when administered alone). As used herein, “pain” includes all types of pain, including pain in both the peripheral and central nervous systems. The present invention, therefore, provides potent analgesics that are effective for the treatment, management, and amelioration of pain, including, but not limited to, inflammatory pain, neuropathic pain, acute pain, traumatic pain, infection-related pain, postoperative or post-procedural pain, nociceptive pain, dental pain, migraine, cluster headaches, tension headaches, neuralgia, cancer pain, resistant pain, pain resulting from burns, labor and delivery pain, postpartum pain, irritable bowel syndrome, fibromyalgia, pancreatic pain, myocardial infarction pain, temporal-mandibulla disorders, including both pain in the central nervous system as well as pain in the peripheral nervous system, chronic pain, and regional and generalized pain syndromes, and reduces the likelihood of adverse effects, such as, but not limited to, drowsiness, intestinal problems, development of physical dependence, and tolerance, etc. The terms “alleviating”, “suppressing” and “inhibiting” refer to any indicia of success in the treatment or alleviating of pain, including both objective and subjective parameters such as abatement, diminishing of symptoms, making the pain symptom or condition more tolerable to the patient or subject, decreasing the frequency or duration of the pain, or preventing or decreasing the onset of pain expected to occur after an event.

The present invention encompasses certain combinations of centrally acting (i.e., cross the blood brain barrier) κ-opioid receptor agonists and pure opioid antagonists (preferably, non-selective opioid antagonists that antagonize at least κ, μ, and δ opioid receptors) that produce analgesia, but would induce anti-analgesia or produce less analgesia when the agonist or the antagonist is administered alone. The present invention teaches methods and compositions that use κ-opioid receptor agonists to effectively treat pain with fewer side effects associated with administration of μ receptor opioid agonists, such as, but not limited to dysphoria and potential for addiction or dependency on the μ opioid.

Further, in certain embodiments, the present invention encompasses certain combinations of κ-opioid receptor agonists and opioid antagonists that produce enhanced analgesia in both male and female subjects whereas administration of the κ-opioid agonist alone in human and/or animal subjects (preferably human) produces gender dimorphic effects, i.e. produces significantly more analgesia in females than males or produces analgesia in females and anti-analgesia in males, in groups comprising 10, 15, 20, 25, 30, or more subjects.

### Analgesic Compositions

The present invention provides methods for the treatment of pain and analgesic pharmaceutical compositions comprising combinations of centrally-acting-opioid receptor agonist (preferably, exhibiting gender dimorphism at certain doses) and centrally-acting, preferably non-selective opioid antagonists that provide greater pain relief than achieved with administration of either the agonist or antagonist alone (or, in certain embodiments, greater than the additive analgesic effects of the agonist and antagonist administered alone). Suitable κ-opioid receptor agonists include, but are not limited to benzomorphan derivatives, such as, but not limited to, nalbuphine, butorphanol, ketazocine, ethylketazocine, pentazocine, dezocine, and bremazocine; a benzacetamide derivative, such as, but not limited to U50,488, U69,593, U62,066 (spiradoline), PD 117302, CI-977, DuP 747, ICI 197067, ICI 199441, BRL 52537A, or BRL 52656A; a phenothiazine derivative, such as but not limited to Rp 60180; a thiazine derivative, such as, but not limited to R-84760; or a benzodiazepine derivative, such as, but not limited to Tifluadom. In preferred embodiments of the invention, the κ-opioid agonist is butorphanol or pentazocine, or, most preferably, nalbuphine. Suitable opioid antagonists include, but are not limited to nalorphine, levalorphan, oxylorphan, cyprenorphine, preferably, naltrexone, methylnaltrexone and nalnefene, or, most preferably, naloxone.

The κ-opioid receptor agonists and opioid antagonists of this invention may be in any pharmaceutically acceptable form, e.g., in the form of the free base compound, a pharmaceutically acceptable salt, a prodrug, or a mixture thereof. As used herein and unless otherwise indicated, the term “free base” of the κ-opioid agonist or opioid antagonist refers to the pure form of the respective agonist or antagonist. As used herein and unless otherwise indicated, the term “pharmaceutically acceptable” means approved by a regulatory agency of the Federal, state, or other foreign government or listed in the U.S. Pharmacopoeia or other generally recognized pharmacopeias for use in animals, more particularly humans.

The term “pharmaceutically acceptable salts” is meant to include salts of the active compounds which are prepared with relatively nontoxic acids or bases, depending on the particular substituents found on the compounds described herein. By “pharmaceutically acceptable” is meant that the salt in question is or can be approved by a regulatory agency of the Federal, state, or other foreign government or listed in the U.S. Pharmacopoeia or other generally recognized pharmacopeias for use in animals, more particularly in humans. When compounds of the present invention contain relatively acidic functionalities, base addition salts can be obtained by contacting the neutral form of such compounds with a sufficient amount of the desired base, either neat or in a suitable inert solvent.

Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts”, Journal of Pharmaceutical Science, 1977, 66, 1-19, the text of which is hereby incorporated herein, in its entirety).

The κ-opioid agonist and the antagonist of the invention may be in the form of the free base, a salt, a prodrug, or a mixture of two or more of these. Although certain specific embodiments of the invention provide amounts and/or dosages for a specific form of the κ-opioid agonist and/or the opioid antagonist, i.e., the agonist or the antagonist free base, a pharmaceutically acceptable salt, prodrug, or mixture thereof, the equivalent amount of any form of the agonist or antagonist may be used in this invention. As used herein and unless otherwise indicated, the term “equivalent amount” or “amount equivalent to” is defined as the amount of agonist or antagonist free base, salt, prodrug, or mixture (or any other pharmaceutically acceptable form) thereof that contains the same weight of the active ingredient (i.e., the same number of moles of naloxone, nalbuphine or other opioid antagonists and κ-opioid agonists, etc.), or produces the same blood concentration of the respective agonist or antagonist or active metabolite of agonist or antagonist as produced by an amount of the agonist or antagonist in specified form. In certain embodiments, for example, approximately 1.0 mg of naloxone hydrochloride salt contains approximately 0.9 mg of naloxone free base and approximately 0.9 mg of naloxone free base produces the same blood concentration of naloxone or active metabolite of naloxone when administered intravenously as does intravenous administration of approximately 1 mg of naloxone hydrochloride salt, therefore, an equivalent amount of naloxone free base to 1 mg of naloxone hydrochloride salt would be approximately 0.9 mg.

The actual equivalent amounts of one form to another form may depend on how the respective forms are administered due to different absorption rates of the agonist or antagonist based the on method of administration. Unless otherwise indicated, the different forms of the agonist or antagonist are administered by the same method. If the different forms of the agonist or antagonist are administered by different methods, the equivalent amounts of the forms are amounts that produce the same blood concentration of the respective agonist or antagonist, or active metabolite of the agonist or antagonist. In certain embodiments, an equivalent amount includes an amount of the agonist and/or antagonist that is released over a period of time, e.g., 15 minutes, 30 minutes, 1 hour, 2 hours, 4 hours, 6 hours, 8 hours, 10 hours, 12 hours, or 24 hours, into the site of delivery, even if the administration does not achieve the same blood concentration of the agonist and/or antagonist.

Methods of determining equivalent amounts of one form to another form, for example, naloxone hydrochloride salt to naloxone free base, are readily known in the art. Any method commonly known in the art may be used to measure the blood concentration of the κ-opioid agonist, the opioid antagonist, or active metabolites of the agonist or antagonist. For example, methods for measuring the blood concentration of opioids, such as nalbuphine, and of opioid antagonists are well-known in the art (see, e.g., Pao et al., 2000, “High-performance liquid chromatographic method for the simultaneous determination of nalbuphine and its prodrug, sebacoyl dinalbuphine ester, in dog plasma and application to pharmacokinetic studies in dogs,” J. Chromatogr. B Biomed. Sci. Appl. 746(2):241-7; Sung et al., 2000, “Delivery of nalbuphine and its prodrugs across skin by passive diffusion and iontophoresis,” J. Control. Release 67(1): 1-8; de Cazanove et al., 1997, “Determination of nalbuphine in human plasma by high-performance liquid chromatography with electrochemical detection. Application to pharmacokinetic study,” J. Chromatogr. B Biomed. Sci. Appl. 690(1-2): 203-10; Ho et al., 1996 Apr. 12, “Determination of nalbuphine by high-performance liquid chromatography with ultraviolet detection: application to human and rabbit pharmacokinetic studies,” J. Chromatogr. B Biomed. Appl. 678(2): 289-96; Nicolle et al., 1995 Jan. 6, “Rapid and sensitive high-performance liquid chromatographic assay for nalbuphine in plasma,” J. Chromatogr. B Biomed. Appl. 663(1):111-7; Wetzelsberger et al., 1988 December, “Internally standardized method for the determination of nalbuphine in human plasma by means of high performance liquid chromatography with electrochemical coulometric detection,” Arzneimittelforschung 38(12):1768-71; Dube et al., 1988 May 13, “Determination of nalbuphine by high-performance liquid chromatography with electrochemical detection: application to clinical samples from postoperative patients,” J. Chromatogr. 427(1):113-20; Lo et al., 1987 November, “The disposition and bioavailability of intravenous and oral nalbuphine in healthy volunteers,” J. Clin. Pharmacol. 27(11): 866-73; incorporated herein by reference in their entireties; Ameyibor et al., 1997 Dec. 5, “Resolution and quantitation of pentazocine enantiomers in human serum by reversed-phase high-performance liquid chromatography using sulfated beta-cyclodextrin as chiral mobile phase additive and solid-phase extraction,” J. Chromatogr. B. Biomed. Sci. Appl. 703(1-2):273; Suzuki et al., 1997 November, “Pharmacokinetics of pentazocine and its occupancy of opioid receptors in rat brain,” Biol. Pharm. Bull. 20(11):1193-8; Kelly et al., 1994 September-October, “HPLC separation of pentazocine enantiomers in serum using an ovomucoid chiral stationary phase,” Biomed. Chromatogr. 8(5):255-7; Misztal et al., 1991 June, “Determination of pentazocine in human plasma by high performance liquid chromatography,” Pharmazie, 46(6): 464-5; Moeller et al., 1990 Aug. 24, “High-performance liquid chromatographic determination of pentazocine in plasma,” J. Chromatogr. 530(1):200-5; Kintz et al., 1990 Apr., “Simultaneous screening and quantification of several nonopiate narcotic analgesics and phencyclidine in human plasma using capillary gas chromatography,” Methods Find Exp. Clin. Pharmacol. 12(3):193-6; Shibanoki et al., 1987 Oct. 30, “Application of high-performance liquid chromatography with electrochemical detection for monitoring the concentration of pentazocine in human blood,” J. Chromatogr. 421(2):425-9; Anderson et al., 1982 Jan. 8, “High-performance liquid chromatographic analysis of pentazocine in blood and plasma,” J. Chromatogr. 227(1): 239-43; Clemans et al., 1979 May, “Plasma pentazocine radioimmunoassay,” J. Pharm. Sci. 68(5): 626-8; and Williams et al., 1974 Jan., “Pentazocine radioimmunoassay,” 7(1):119-43.

Certain embodiments of the invention may include a pharmaceutically acceptable carrier. The term “carrier” refers to a diluent, adjuvant (e.g., Freund's adjuvant (complete or incomplete)), excipient, or vehicle with which the therapeutic is administered. Such pharmaceutical carriers can be sterile liquids, such as water and oils, including those of petroleum, animal, vegetable, or synthetic origin, such as peanut oil, soybean oil, mineral oil, sesame oil, and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica, gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. These compositions can take the form of solutions, suspensions, emulsions, tablets, pills, capsules, powders, sustained-release formations and the like. Oral formulations can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

The invention encompasses pharmaceutical compositions comprising an amount of a κ-opioid receptor agonist and opioid antagonist that produces greater analgesia than the administration of either the agonist or antagonist alone (or greater than their additive effects) together with a suitable amount of carrier so as to provide the form for proper administration to the patient. The formulation of the invention should suit the mode of administration. The amount of the compositions of the invention which will be effective in treating, managing, or ameliorating pain can be determined by standard clinical techniques. The precise dose to be employed in the formulation will depend on the route of administration, the intensity of pain the subject is experiencing, and the size and weight of the subject, the subject gender and should be decided according to the judgment of the practitioner and each patient's circumstances.

In certain preferred embodiments of the invention, the pharmaceutical compositions comprise an amount of a κ-opioid receptor agonist and an amount of an opioid receptor antagonist, such that the combination provides greater analgesia than either agonist or antagonist alone and/or has reduced side effects compared to either agonist or antagonist alone. The opioid antagonist is preferably naloxone.

More specifically, preferred pharmaceutical compositions of this invention are formulated for intravenous administration or mucosal administration (e.g., nasal or pulmonary administration) and comprise of naloxone hydrochloride salt in amounts of 0.02 mg to 8 mg, 0.02 to 7 mg, 0.02 mg to 6 mg, 0.02 to 5 mg, 0.02 to 4 mg, 0.02 to 3 mg, 0.02 to 2 mg, 0.02 to 2 mg, 0.1 mg to 8 mg, 0.1 mg to 7 mg, 0.1 mg to 6 mg, 0.1 mg to 5 mg, 0.1 mg to 4 mg, 0.1 mg to 3 mg, 0.1 mg to 2 mg, 0.1 mg to 1 mg, 0.2 mg to 8 mg, 0.2 mg to 7 mg, 0.2 mg to 6 mg, 0.2 mg to 4 mg, 0.2 mg to 3 mg, 0.2 mg to 2 mg, 0.2 mg to 1 mg, 0.4 mg to 8 mg, 0.4 to 7 mg, 0.4 to 6 mg, 0.4 mg to 5 mg, 0.4 mg to 4 mg, 0.4 mg to 3 mg, 0.4 mg to 2 mg, 0.4 mg to 1 mg, 0.5 mg to 8 mg, 0.5 mg to 6 mg, 0.5 mg to 5 mg, 0.5 mg to 4 mg, 0.5 mg to 3 mg, 0.5 mg to 3 mg, 0.5 to 2 mg, 0.5 mg to 1 mg, 1 mg to 8 mg, 1 mg to 6 mg, 1 mg to 5 mg, 1 mg to 4 mg, 1 mg to 3 mg, 1 mg to 2 mg, 5 mg to 8 mg, 4 mg to 7 mg, 3 mg to 5 mg, 0.02 mg to 1 mg, preferably 0.1 mg to 0.8 mg, 0.2 mg to 0.8 mg, 0.3 mg to 0.8 mg, 0.4 mg to 0.8 mg, 0.5 mg to 0.8 mg, 0.1 mg to 0.7 mg, 0.2 mg to 0.7 mg, 0.3 mg to 0.7 mg, 0.4 mg to 0.7 mg, 0.1 mg to 0.6 mg, 0.2 mg to 0.6 mg, 0.3 to 0.6 mg, 0.1 mg to 0.5 mg, 0.15 mg to 0.5 mg, 0.2 mg to 0.5 mg, 0.25 mg to 0.5 mg, 0.3 mg to 0.5 mg, 0.35 mg to 0.5 mg, 0.1 mg to 0.45 mg, 0.15 mg to 0.45 mg, 0.2 mg to 0.45 mg, 0.25 mg to 0.45 mg, 0.1 mg to 0.3 mg, 0.13 mg to 0.3 mg, 0.2 mg to 0.3 mg, 0.1 mg to 0.25 mg, 0.15 mg to 0.25 mg, 0.1 mg to 0.2 mg, 0.1 mg to 0.15 mg. Typical amounts of naloxone hydrochloride salt are 0.1 mg, 0.15 mg, 0.2 mg, 0.25 mg, 0.3 mg, 0.35 mg, 0.4 mg, 0.45 mg, 0.5 mg, 0.55 mg, 0.6 mg 0.7 mg, 0.75 mg, 0.8 mg, 1 mg, 1.6 mg, 2 mg, 2.4 mg, 3 mg, 4 mg, 5 mg, 6 mg, 7 mg and 8 mg naloxone hydrochloride salt. The pharmaceutical compositions of the preferred embodiments of the invention may alternatively comprise of an equivalent amount of naloxone free base, non-hydrochloride salt, prodrug or mixture thereof to the recited amounts of naloxone hydrochloride salt.

The present invention also provides pharmaceutical compositions formulated for administration by a method other than intravenous administration. In these preferred embodiments, the pharmaceutical composition comprises an amount of naloxone free base, pharmaceutically acceptable salt, prodrug, or mixture thereof equivalent (i.e., resulting in the same blood concentration) as intravenous administration of 0.02 mg to 8 mg, 0.02 to 7 mg, 0.02 mg to 6 mg, 0.02 to 5 mg, 0.02 to 4 mg, 0.02 to 3 mg, 0.02 to 2 mg, 0.02 to 2 mg, 0.1 mg to 8 mg, 0.1 mg to 7 mg, 0.1 mg to 6 mg, 0.1 mg to 5 mg, 0.1 mg to 4 mg, 0.1 mg to 3 mg, 0.1 mg to 2 mg, 0.1 mg to 1 mg, 0.2 mg to 8 mg, 0.2 mg to 7 mg, 0.2 mg to 6 mg, 0.2 mg to 4 mg, 0.2 mg to 3 mg, 0.2 mg to 2 mg, 0.2 mg to 1 mg, 0.4 mg to 8 mg, 0.4 to 7 mg, 0.4 to 6 mg, 0.4 mg to 5 mg, 0.4 mg to 4 mg, 0.4 mg to 3 mg, 0.4 mg to 2 mg, 0.4 mg to 1 mg, 0.5 mg to 8 mg, 0.5 mg to 6 mg, 0.5 mg to 5 mg, 0.5 mg to 4 mg, 0.5 mg to 3 mg, 0.5 mg to 3 mg, 0.5 to 2 mg, 0.5 mg to 1 mg, 1 mg to 8 mg, 1 mg to 6 mg, 1 mg to 5 mg, 1 mg to 4 mg, 1 mg to 3 mg, 1 mg to 2 mg, 5 mg to 8 mg, 4 mg to 7 mg, 3 mg to 5 mg, 0.02 mg to 1 mg, preferably 0.1 mg to 0.8 mg, 0.2 mg to 0.8 mg, 0.3 mg to 0.8 mg, 0.4 mg to 0.8 mg, 0.5 mg to 0.8 mg, 0.1 mg to 0.7 mg, 0.2 mg to 0.7 mg, 0.3 mg to 0.7 mg, 0.4 mg to 0.7 mg, 0.1 mg to 0.6 mg, 0.2 mg to 0.6 mg, 0.3 to 0.6 mg, 0.1 mg to 0.5 mg, 0.15 mg to 0.5 mg, 0.2 mg to 0.5 mg, 0.25 mg to 0.5 mg, 0.3 mg to 0.5 mg, 0.35 mg to 0.5 mg, 0.1 mg to 0.45 mg, 0.15 mg to 0.45 mg, 0.2 mg to 0.45 mg, 0.25 mg to 0.45 mg, 0.1 mg to 0.3 mg, 0.13 mg to 0.3 mg, 0.2 mg to 0.3 mg, 0.1 mg to 0.25 mg, 0.15 mg to 0.25 mg, 0.1 mg to 0.2 mg, 0.1 mg to 0.15 mg naloxone hydrochloride salt.

In certain embodiments, the pharmaceutical composition of the present invention comprises an amount of nalbuphine free base, pharmaceutically acceptable salt, prodrug, or mixture thereof equivalent (i.e., resulting in the same blood concentration) as intravenous administration of 1 mg to 50 mg, 1 mg to 45 mg, 1 mg to 40 mg, 1 mg to 35 mg, 1 mg to 30 mg, 1 mg to 25 mg, 1 mg to 20 mg, 1 mg to 15 mg, 1 mg to 10 mg, 1 mg to 9 mg, 1 mg to 8 mg, 2 mg to 8 mg, 3 mg to 8 mg, 1 mg to 7 mg, 2 mg to 7 mg, 3 mg to 7 mg, 4 mg to 7 mg, 1 mg to 6 mg, 2 mg to 6 mg, 3 mg to 6 mg, 1 mg to 5 mg, 2 mg to 5 mg, 1 mg to 4 mg, 1 mg to 3 mg, 1 mg to 2 mg nalbuphine hydrochloride salt.

Any method in the art may be used to measure the blood concentration. For example, but not by way of limitation, blood concentration of κ-opioid agonists and opioid antagonists may be measured by high-performance liquid chromatography or any assays described, supra. Blood concentration of κ-opioid agonists and antagonists may be measured immediately after administration, less than or at approximately 10 minutes after administration, less than or at approximately 15 minutes after administration, less than or at approximately 20 minutes after administration, less than or at approximately 30 minutes after administration, less than or at approximately 45 minutes after administration, less than or at approximately 1 hour after administration, less than or at approximately 2 hours after administration, less than or approximately 3 hours after administration, less than or approximately 4 hours after administration, less than or approximately 5 hours after administration, or less than or approximately 6 hours after administration. Depending on the specific non-intravenous mode of administration, a composition for non-intravenous administration may comprise a ratio of the κ-opioid agonist to opioid antagonist that is lesser, equal, or greater than the ratio of κ-opioid agonist to opioid agonist in an equivalent composition for intravenous administration. Also depending on the specific non-intravenous mode of administration, a composition for administration other than intravenous may comprise lesser, equal, or greater amounts of κ-opioid agonist and opioid antagonist, by weight, than an equivalent composition for intravenous administration.

In certain preferred embodiments of the invention, pharmaceutical compositions formulated for sublingual administration, the κ-opioid receptor agonist is nalbuphine hydrochloride salt and the opioid antagonist is naloxone hydrochloride salt and the composition comprises 0.1 mg to 10 mg, 0.1 mg to 9 mg, 0.1 mg to 8 mg, 0.1 mg to 7 mg, 0.1 mg to 6 mg, 0.2 mg to 10 mg, 0.2 mg to 9 mg, 0.2 mg to 8 mg, 0.2 mg to 7 mg, 0.2 mg to 6 mg, 0.2 mg to 5 mg, 0.3 mg to 10 mg, 0.3 mg to 9 mg, 0.3 mg to 7 mg, 0.3 mg to 6 mg, 0.3 mg to 5 mg, 0.3 mg to 4 mg, 0.4 mg to 4 mg, 0.5 mg to 4 mg, 0.4 mg to 3.5 mg, 0.5 mg to 3.5 mg, 0.6 mg to 3.5 mg, 0.4 mg to 3 mg, 0.5 mg to 3 mg, 0.4 mg to 2.8 mg, 0.6 mg to 2.7 mg, 0.4 mg to 2.5 mg, 0.5 mg to 2.2 mg, 0.4 mg to 2 mg, 0.6 mg to 2 mg, 0.4 mg to 1.5 mg, or 0.4 mg to 1 mg naloxone hydrochloride salt or equivalent amounts of naloxone free base, non-chloride salt, prodrug, or mixture thereof.

If the pharmaceutical composition formulated for sublingual administration comprises pentazocine hydrochloride salt as the K-Opioid receptor agonist and naloxone hydrochloride salt as the opioid antagonist, the amount of naloxone hydrochloride salt is preferably 1 mg to 10 mg, 1 mg to 9 mg, 1 mg to 8 mg, 1 mg to 7 mg, 1 mg to 6 mg, 1 mg to 5 mg, 1 mg to 4 mg, 2 mg to 10 mg, 2 mg to 9 mg, 2 mg to 8 mg, 2 mg to 7 mg, 2 mg to 6 mg, 2 mg to 5 mg, 2 mg to 4 mg, 2 mg to 3.8 mg, 2 mg to 3.6 mg, 2 mg to 3.4 mg, 2 mg to 3.4 mg, 2.2 mg to 3.5 mg, 2 mg to 3.2 mg, 3 mg to 4 mg, or 2 mg to 3 mg or equivalent amounts of naloxone free base, non-chloride salt, prodrug, or mixture thereof.

In other specific embodiments of the present invention, pharmaceutical compositions formulated for sublingual administration in which butorphanol tartrate salt is the κ-opioid receptor agonist and naloxone is the opioid antagonist, comprise 0.1 to 4 mg, 0.1 mg to 3.5 mg, 0.1 mg to 3 mg, 0.1 to 2.5 mg, 0.1 mg to 2 mg, 0.1 mg to 1 mg, 0.3 mg to 0.8 mg, or 0.1 mg to 0.8 mg of naloxone hydrochloride salt or equivalent amounts of naloxone free base, non-hydrochloride chloride salt, prodrug, or mixture thereof.

In general, the amounts of κ-opioid agonist in the pharmaceutical compositions of this invention are in the range of 5% to 100% of the recommended analgesic dose (e.g., as provided in the Physician's Desk Reference or other commonly used reference). Preferably, the dose the κ-opioid agonist is 5% to 90%, 10% to 90%, 5% to 85%, 10% to 85%, 15% to 85%, 20% to 80%, 5% to 75%, 10% to 75%, 15% to 70%, 15% to 60%, 5% to 55%, 10% to 55%, 15% to 50%, 5% to 50%, 5% to 45%, 5% to 40%, 5% to 35%, 5% to 30%, 10% to 40%, 10% to 35%, 10% to 30%, 15% to 40%, 15% to 35%, 15% to 30%, 5% to 25%, 10% to 25%, 15% to 25%, 5% to 20%, and 5% to 15% of the recommended analgesic dose. However, as mentioned above, the compositions may contain a fraction or a multiple of the recommended analgesic dose.

In a specific embodiment of the invention, the pharmaceutical composition is an intravenous formulation or mucosal formulation comprising a κ-opioid receptor agonist and an opioid antagonist that produces more analgesia and/or reduced side effects than either the agonist or antagonist alone. In preferred embodiments, the pharmaceutical composition comprises of an amount of an opioid antagonist hydrochloride salt, preferably naloxone hydrochloride salt, and 6.25 to 49 times greater, 6.25 to 40 times greater, 6.25 to 35 times greater, 6.25 to 30 times greater, 6.25 to 20 times greater, 8 to 35 times greater, 8 to 30 times greater, 8 to 25 times greater, 10 to 30 times greater, 10 to 20 times greater, 15 to 25 times greater, 20 to 25 times greater, 10 to 15 times greater, 9 to 15 times greater, 5 to 10 times greater, 30 to 49 times greater, 20 to 30 times greater, 15 to 20 times greater, 13 to 15 times greater, 11 to 13 times greater, 9 to 11 times greater, 7 to 9 times greater, or 6.25 to 7 times greater by weight, nalbuphine hydrochloride salt than the amount of opioid antagonist hydrochloride salt. Non-limiting examples of intravenous formulations of the present invention are 3 mg of nalbuphine hydrochloride salt with 0.4 mg naloxone hydrochloride salt (i.e., 7.5 times greater nalbuphine hydrochloride salt than the opioid antagonist hydrochloride salt; 1.25 mg nalbuphine hydrochloride salt with 0.1 mg naloxone hydrochloride salt, 2.5 mg nalbuphine hydrochloride salt with 0.2 mg naloxone hydrochloride salt, 5 mg nalbuphine hydrochloride salt with 0.4 mg naloxone hydrochloride salt, 10 mg nalbuphine hydrochloride salt with 0.8 mg naloxone hydrochloride salt, 20 mg nalbuphine hydrochloride salt with 1.6 mg naloxone hydrochloride salt, 25 mg nalbuphine hydrochloride salt with 2.0 mg naloxone hydrochloride salt, and 30 mg nalbuphine hydrochloride salt with 2.4 mg naloxone hydrochloride salt (i.e., 12.5 times greater, by weight, nalbuphine hydrochloride salt than the naloxone hydrochloride salt); 5 mg nalbuphine hydrochloride salt with 0.2 mg naloxone hydrochloride salt and 10 mg nalbuphine hydrochloride salt with 0.4 mg naloxone hydrochloride salt (i.e., 25 times greater nalbuphine hydrochloride salt than the naloxone hydrochloride salt); 4.9 mg nalbuphine hydrochloride salt with 0.1 mg naloxone hydrochloride salt and 9.8 mg nalbuphine hydrochloride salt with 0.2 mg naloxone hydrochloride salt (i.e., 49 times greater, by weight, nalbuphine hydrochloride salt than naloxone hydrochloride salt).

Alternatively, preferred embodiments of pharmaceutical compositions for intravenous administration comprise 1 mg to 50 mg, 1 mg to 45 mg, 1 mg to 40 mg, 1 mg to 35 mg, 1 mg to 30 mg, 1 mg to 25 mg, 1 mg to 20 mg, 1 mg to 15 mg, 1 mg to 10 mg, 1 mg to 9 mg, 1 mg to 8 mg, 2 mg to 8 mg, 3 mg to 8 mg, 1 mg to 7 mg, 2 mg to 7 mg, 3 mg to 7 mg, 4 mg to 7 mg, 1 mg to 6 mg, 2 mg to 6 mg, 3 mg to 6 mg, 1 mg to 5 mg, 2 mg to 5 mg, 1 mg to 4 mg, 1 mg to 3 mg, 1 mg to 2 mg nalbuphine hydrochloride salt. In certain preferred embodiments in which nalbuphine hydrochloride salt is the κ-opioid agonist, preferably, 0.02 mg to 8 mg, preferably, 0.1 mg to 0.8 mg naloxone hydrochloride salt is the opioid antagonist hydrochloride salt. Non-limiting examples of the method of the present invention comprises 1 mg, 1.25 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 15 mg, 20 mg, 25 mg, 30 mg, 40 mg, or 50 mg of nalbuphine hydrochloride salt with 0.1 mg, 0.15 mg, 0.2 mg, 0.2 mg, 0.25 mg, 0.3 mg, 0.35 mg, 0.4 mg, 0.5 mg, 0.6 mg, 0.7 mg, 0.8 mg, 1 mg, 1.6 mg, 2 mg, 2.4 mg, 3 mg, 4 mg, 5 mg, 6 mg, 7 mg and 8 mg of naloxone hydrochloride salt.

For example, specific embodiments of the invention comprise of intravenous administration of 0.1 mg naloxone hydrochloride salt with 1.25 mg nalbuphine hydrochloride salt, 0.2 mg of naloxone hydrochloride salt with 1 mg nalbuphine hydrochloride salt, 0.2 mg of naloxone hydrochloride salt with 1.5 mg nalbuphine hydrochloride salt, 0.2 mg of naloxone hydrochloride salt with 2 mg nalbuphine hydrochloride salt, 0.2 mg of naloxone hydrochloride salt with 2.5 mg nalbuphine hydrochloride salt, 0.2 mg of naloxone hydrochloride salt with 3 mg nalbuphine hydrochloride salt, 0.2 mg of naloxone hydrochloride salt with 3.5 mg nalbuphine hydrochloride salt, 0.2 mg of naloxone hydrochloride salt with 4 mg nalbuphine hydrochloride salt, 0.2 mg of naloxone hydrochloride salt with 4.5 mg nalbuphine hydrochloride salt, 0.2 mg of naloxone hydrochloride salt with 5 mg nalbuphine hydrochloride salt, 0.2 mg of naloxone hydrochloride salt with 5.5 mg nalbuphine hydrochloride salt, 0.2 mg of naloxone hydrochloride salt with 6 mg nalbuphine hydrochloride salt, 0.2 mg of naloxone hydrochloride salt with 6.5 mg nalbuphine hydrochloride salt, 0.2 mg of naloxone hydrochloride salt with 7 mg nalbuphine hydrochloride salt 0.2 mg of naloxone hydrochloride salt with 7.5 mg nalbuphine hydrochloride salt, 0.2 mg of naloxone hydrochloride salt with 8 mg nalbuphine hydrochloride salt, 0.2 mg of naloxone hydrochloride salt with 8.5 mg nalbuphine hydrochloride salt, 0.2 mg of naloxone hydrochloride salt with 9 mg nalbuphine hydrochloride salt, 0.2 mg of naloxone hydrochloride salt with 9.5 mg nalbuphine hydrochloride salt, 0.2 mg of naloxone hydrochloride salt with 10 mg nalbuphine hydrochloride salt, 0.4 mg of naloxone hydrochloride salt with 1 mg nalbuphine hydrochloride salt, 0.4 mg of naloxone hydrochloride salt with 1.5 mg nalbuphine hydrochloride salt, 0.4 mg of naloxone hydrochloride salt with 2.0 mg nalbuphine hydrochloride salt, 0.4 mg of naloxone hydrochloride salt with 2.5 mg nalbuphine hydrochloride salt, 0.4 mg of naloxone hydrochloride salt with 3 mg nalbuphine hydrochloride salt, 0.4 mg of naloxone hydrochloride salt with 3.5 mg nalbuphine hydrochloride salt, 0.4 mg of naloxone hydrochloride salt with 4 mg nalbuphine hydrochloride salt, 0.4 mg of naloxone hydrochloride salt with 4.5 mg nalbuphine hydrochloride salt, 0.4 mg of naloxone hydrochloride salt with 5 mg nalbuphine hydrochloride salt, 0.4 mg of naloxone hydrochloride salt with 5.5 mg nalbuphine hydrochloride salt, 0.4 mg of naloxone hydrochloride salt with 6 mg nalbuphine hydrochloride salt, 0.4 mg of naloxone hydrochloride salt with 6.5 mg nalbuphine hydrochloride salt, 0.4 mg of naloxone hydrochloride salt with 7 mg nalbuphine hydrochloride salt, 0.4 mg of naloxone hydrochloride salt with 7.5 mg nalbuphine hydrochloride salt, 0.4 mg of naloxone hydrochloride salt with 8 mg nalbuphine hydrochloride salt, 0.4 mg of naloxone hydrochloride salt with 8.5 mg nalbuphine hydrochloride salt, 0.4 mg of naloxone hydrochloride salt with 9 mg nalbuphine hydrochloride salt, 0.4 mg of naloxone hydrochloride salt with 9.5 mg nalbuphine hydrochloride salt, 0.4 mg of naloxone hydrochloride salt with 10 mg nalbuphine hydrochloride salt, and 0.8 mg naloxone hydrochloride salt with 10 mg nalbuphine hydrochloride salt. The present invention further encompasses pharmaceutical compositions comprising nalbuphine free base, non-hydrochloride salt, prodrug, or mixture thereof in equivalent amounts to nalbuphine hydrochloride salt intravenously administered, provided that the composition does not comprise 5 mg of nalbuphine free base is not combined with 0.4 mg of naloxone free base when the composition is administered intravenously. In other embodiments, the invention provides, for example, controlled or sustained release formulations that release equivalent amounts of nalbuphine and naloxone to the site of administration (e.g., the oral cavity) over a period of 15 minutes, 30 minutes, 1 hour, 2 hours, 4 hours, 6 hours, 8 hours, 10 hours, 12 hours, or 24 hours.

In another embodiment of the invention, the pharmaceutical composition for intravenous administration or mucosal administration (e.g., nasal or pulmonary administration) comprises pentazocine free base, prodrug, non-hydrochloride salt, most preferably in the hydrochloride salt, or mixture thereof, is administered as the κ-opioid receptor agonist with the opioid antagonist, preferably naloxone, to treat, manage, or ameliorate pain. The amount of pentazocine hydrochloride salt in the intravenous formulation is preferably 18 to 120 times greater, 25 to 120 times greater, 18 to 110 times greater, 25 to 110 times greater. 18 to 100 times greater, 25 to 100 times greater, 18 to 95 times greater, 25 to 90 times greater, 30 to 90 times greater, 35 to 90 times greater, 18 to 85 times greater, 20 to 80 times greater, 20 to 60 times greater, 20 to 50 times greater, 25 to 55 times greater, 35 to 80 times greater, 20 to 75 times greater, 25 to 70 times greater, 40 to 100 times greater, 50 to 100 times greater, 55 to 95 times greater, 45 to 90 times greater, 40 to 70 times greater, 18 to 50 times greater, 18 to 40 times greater, or 18 to 35 times greater, 18 to 30 times greater, by weight, pentazocine hydrochloride salt than the opioid antagonist hydrochloride salt, preferably, naloxone hydrochloride salt.

Examples of intravenous formulations and mucosal formulations of the present invention are, but not limited to, 10 mg of pentazocine hydrochloride salt with 0.4 mg of naloxone hydrochloride salt (i.e., 25 times greater, by weight, pentazocine hydrochloride salt than naloxone hydrochloride salt), 15 mg of pentazocine hydrochloride salt with 0.3 mg naloxone hydrochloride salt (i.e., 30 times greater, by weight, pentazocine hydrochloride salt than naloxone hydrochloride salt), and 25 mg of pentazocine hydrochloride salt with 0.5 mg naloxone hydrochloride salt (i.e., 50 times greater pentazocine hydrochloride salt than naloxone hydrochloride salt). In preferred embodiments of the invention, the amounts of pentazocine hydrochloride salt in the pharmaceutical composition, by weight, are 3 mg to 50 mg, 4 mg to 50 mg, 5 mg to 50 mg, 6 mg to 50 mg, 7 mg to 50 mg, 3 mg to 45 mg, 5 mg to 45 mg, 10 mg to 45 mg, 15 mg to 45 mg, 5 mg to 40 mg, 10 mg to 40 mg, 3 mg to 35 mg, 4 mg to 35 mg, 5 mg to 35 mg, 10 mg to 35 mg, 3 mg to 30 mg, 4 mg to 30 mg, 5 mg to 30 mg, 3 mg to 25 mg, 4 mg to 25 mg, 3 mg to 20 mg, 4 mg to 20 mg, 5 mg to 25 mg, 10 mg to 25 mg, 15 mg to 30 mg, 15 mg to 25 mg, 10 mg to 20 mg, and 10 mg to 15 mg.

In certain embodiments in which pentazocine hydrochloride salt is the κ-opioid agonist, the composition comprises 0.02 mg to 8 mg, 0.02 to 7 mg, 0.02 mg to 6 mg, 0.02 to 5 mg, 0.02 to 4 mg, 0.02 to 3 mg, 0.02 to 2 mg, 0.02 to 2 mg, 0.1 mg to 8 mg, 0.1 mg to 7 mg, 0.1 mg to 6 mg, 0.1 mg to 5 mg, 0.1 mg to 4 mg, 0.1 mg to 3 mg, 0.1 mg to 2 mg, 0.1 mg to 1 mg, 0.2 mg to 8 mg, 0.2 mg to 7 mg, 0.2 mg to 6 mg, 0.2 mg to 4 mg, 0.2 mg to 3 mg, 0.2 mg to 2 mg, 0.2 mg to 1 mg, 0.4 mg to 8 mg, 0.4 to 7 mg, 0.4 to 6 mg, 0.4 mg to 5 mg, 0.4 mg to 4 mg, 0.4 mg to 3 mg, 0.4 mg to 2 mg, 0.4 mg to 1 mg, 0.5 mg to 8 mg, 0.5 mg to 6 mg, 0.5 mg to 5 mg, 0.5 mg to 4 mg, 0.5 mg to 3 mg, 0.5 mg to 3 mg, 0.5 to 2 mg, 0.5 mg to 1 mg, 1 mg to 8 mg, 1 mg to 6 mg, 1 mg to 5 mg, 1 mg to 4 mg, 1 mg to 3 mg, 1 mg to 2 mg, 5 mg to 8 mg, 4 mg to 7 mg, 3 mg to 5 mg, 0.02 mg to 1 mg, preferably 0.1 mg to 0.8 mg, 0.2 mg to 0.8 mg, 0.3 mg to 0.8 mg, 0.4 mg to 0.8 mg, 0.5 mg to 0.8 mg, 0.1 mg to 0.7 mg, 0.2 mg to 0.7 mg, 0.3 mg to 0.7 mg, 0.4 mg to 0.7 mg, 0.1 mg to 0.6 mg, 0.2 mg to 0.6 mg, 0.3 to 0.6 mg, 0.1 mg to 0.5 mg, 0.15 mg to 0.5 mg, 0.2 mg to 0.5 mg, 0.25 mg to 0.5 mg, 0.3 mg to 0.5 mg, 0.35 mg to 0.5 mg, 0.1 mg to 0.45 mg, 0.15 mg to 0.45 mg, 0.2 mg to 0.45 mg, 0.25 mg to 0.45 mg, 0.1 mg to 0.3 mg, 0.13 mg to 0.3 mg, 0.2 mg to 0.3 mg, 0.1 mg to 0.25 mg, 0.15 mg to 0.25 mg, 0.1 mg to 0.2 mg, 0.1 mg to 0.15 mg of naloxone hydrochloride as the opioid antagonist hydrochloride salt. Non-limiting examples of the present invention are intravenous formulations comprising 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 30 mg, 35 mg, 40 mg, 45 mg, and 50 mg pentazocine hydrochloride salt with 0.1 mg, 0.15 mg, 0.2 mg, 0.2 mg, 0.25 mg, 0.3 mg, 0.35 mg, 0.4 mg, 0.45 mg, 0.5 mg, 0.55 mg, 0.6 mg 0.7 mg, 0.75 mg, 0.8 mg, 1 mg, 1.6 mg, 2 mg, 2.4 mg, 3 mg, 4 mg, 5 mg, 6 mg, 7 mg and 8 mg naloxone hydrochloride salt.

The present invention further encompasses pharmaceutical compositions comprising pentazocine free base, non-hydrochloride salt, prodrug, or mixture thereof in equivalent amounts to pentazocine hydrochloride salt intravenously administered. In other embodiments, the invention provides, for example, controlled or sustained release formulations that release equivalent amounts of pentazocine and naloxone to the site of administration (e.g., the oral cavity) over a period of 15 minutes, 30 minutes, 1 hour, 2 hours, 4 hours, 6 hours, 8 hours, 10 hours, 12 hours, or 24 hours.

In another particular embodiment of the invention, the pharmaceutical composition for intravenous administration comprises butorphanol free base, a pharmaceutically acceptable salt, a prodrug, or a mixture thereof as the κ-opioid receptor agonist. In preferred embodiments of the invention, the pharmaceutical composition for intravenous administration comprises butorphanol tartrate salt in amounts 0.3 to 10 times greater, 0.3 to 9 times greater, 0.3 to 8 times greater, 0.5 to 10 times greater, 0.3 to 7 times greater, 0.5 to 7 times greater, 0.3 to 6 times greater. 0.5 to 6 times greater, 0.3 to 5 times greater, 0.5 to 5 times greater, 0.3 to 4 times greater, 0.5 to 4 times greater, 0.3 to 3 times greater, or 0.5 to 3 times greater, by weight, than the opioid antagonist hydrochloride salt, preferably naloxone hydrochloride salt. Examples of intravenous formulations of the present invention are, but not limited to, 0.3 mg of butorphanol tartrate salt with 0.3 mg of naloxone hydrochloride salt (i.e., 1 time greater, by weight, of butorphanol tartrate salt than naloxone hydrochloride salt), 0.5 mg of butorphanol tartrate salt with 0.2 mg naloxone hydrochloride salt (i.e., 2.5 times greater, by weight, butorphanol tartrate salt than the naloxone hydrochloride salt), and 0.8 mg of butorphanol tartrate salt with 0.4 mg naloxone hydrochloride salt (i.e., 2 times greater, by weight butorphanol tartrate salt than naloxone hydrochloride salt).

Alternatively, the weight of butorphanol tartrate salt in the intravenous formulation or mucosal formulation is preferably 0.2 mg to 2 mg, 0.2 mg to 1.9 mg, 0.2 mg to 1.8 mg, 0.2 mg to 1.7 mg, 0.25 mg to 1.9 mg, 0.25 mg to 1.8 mg, 0.25 mg to 1.75 mg, 0.25 mg to 1.5 mg, 0.25 mg to 1 mg, or 0.2 mg to 1 mg. In certain preferred embodiments of the present invention, the pharmaceutical composition for intravenous administration comprises butorphanol tartrate salt and preferably 0.02 mg to 8 mg, 0.02 to 7 mg, 0.02 mg to 6 mg, 0.02 to 5 mg, 0.02 to 4 mg, 0.02 to 3 mg, 0.02 to 2 mg, 0.02 to 2 mg, 0.1 mg to 8 mg, 0.1 mg to 7 mg, 0.1 mg to 6 mg, 0.1 mg to 5 mg, 0.1 mg to 4 mg, 0.1 mg to 3 mg, 0.1 mg to 2 mg, 0.1 mg to 1 mg, 0.2 mg to 8 mg, 0.2 mg to 7 mg, 0.2 mg to 6 mg, 0.2 mg to 4 mg, 0.2 mg to 3 mg, 0.2 mg to 2 mg, 0.2 mg to 1 mg, 0.4 mg to 8 mg, 0.4 to 7 mg, 0.4 to 6 mg, 0.4 mg to 5 mg, 0.4 mg to 4 mg, 0.4 mg to 3 mg, 0.4 mg to 2 mg, 0.4 mg to 1 mg, 0.5 mg to 8 mg, 0.5 mg to 6 mg, 0.5 mg to 5 mg, 0.5 mg to 4 mg, 0.5 mg to 3 mg, 0.5 mg to 3 mg, 0.5 to 2 mg, 0.5 mg to 1 mg, 1 mg to 8 mg, 1 mg to 6 mg, 1 mg to 5 mg, 1 mg to 4 mg, 1 mg to 3 mg, 1 mg to 2 mg, 5 mg to 8 mg, 4 mg to 7 mg, 3 mg to 5 mg, 0.02 mg to 1 mg, more preferably 0.1 mg to 0.8 mg, 0.2 mg to 0.8 mg, 0.3 mg to 0.8 mg, 0.4 mg to 0.8 mg, 0.5 mg to 0.8 mg, 0.1 mg to 0.7 mg, 0.2 mg to 0.7 mg, 0.3 mg to 0.7 mg, 0.4 mg to 0.7 mg, 0.1 mg to 0.6 mg, 0.2 mg to 0.6 mg, 0.3 to 0.6 mg, 0.1 mg to 0.5 mg, 0.15 mg to 0.5 mg, 0.2 mg to 0.5 mg, 0.25 mg to 0.5 mg, 0.3 mg to 0.5 mg, 0.35 mg to 0.5 mg, 0.1 mg to 0.45 mg, 0.15 mg to 0.45 mg, 0.2 mg to 0.45 mg, 0.25 mg to 0.45 mg, 0.1 mg to 0.3 mg, 0.13 mg to 0.3 mg, 0.2 mg to 0.3 mg, 0.1 mg to 0.25 mg, 0.15 mg to 0.25 mg, 0.1 mg to 0.2 mg, 0.1 mg to 0.15 mg naloxone hydrochloride salt as the opioid antagonist hydrochloride. Non-limiting examples of the present invention are compositions comprising 0.2 mg, 0.3 mg, 0.4 mg, 0.5 mg, 0.6 mg, 0.7 mg, 0.8 mg, 0.9 mg, 1.0 mg, 1.1 mg, 1.1 mg, 1.2 mg, 1.3 mg, 1.4 mg, 1.5 mg, 1.6 mg, 1.7 mg, 1.8 mg, or 2.0 mg of butorphanol tartrate salt with 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.7 mg, 0.75 mg, 0.8 mg, 1 mg, 1.6 mg, 2 mg, 2.4 mg, 3 mg, 4 mg, 5 mg, 6 mg, 7 mg and 8 mg naloxone hydrochloride salt.

The present invention further encompasses pharmaceutical compositions comprising butorphanol free base, non-tartrate salt, prodrug, or mixture thereof in equivalent amounts to butorphanol tartrate salt intravenously administered. In other embodiments, the invention provides, for example, controlled or sustained release formulations that release equivalent amounts of butorphanol and naloxone to the site of administration (e.g., the oral cavity) over a period of 15 minutes, 30 minutes, 1 hour, 2 hours, 4 hours, 6 hours, 8 hours, 10 hours, 12 hours, or 24 hours.

The present invention also includes pharmaceutical compositions for the treatment of pain comprising of a κ-opioid receptor agonist and an opioid antagonist that is to be administered by a method other than by intravenous administration, and in certain embodiments, also other than oral administration for gastro-intestinal uptake. In certain embodiments, the pharmaceutical composition comprises an opioid antagonist hydrochloride salt, preferably naloxone hydrochloride salt, and 6.25 to 49 times greater, 6.25 to 40 times greater, 6.25 to 35 times greater, 6.25 to 30 times greater, 6.25 to 20 times greater, 8 to 35 times greater, 8 to 30 times greater, 8 to 25 times greater, 10 to 30 times greater, 10 to 20 times greater, 15 to 25 times greater, 20 to 25 times greater, 10 to 15 times greater, 9 to 15 times greater, 5 to 10 times greater, 30 to 49 times greater, 20 to 30 times greater, 15 to 20 times greater, 13 to 15 times greater, 11 to 13 times greater, 9 to 11 times greater, 7 to 9 times greater, or 6.25 to 7 times greater, by weight, nalbuphine hydrochloride salt than the opioid antagonist hydrochloride salt.

In specific embodiments in which the pharmaceutical compositions formulated for administration other than intravenous administration, the composition comprises an amount of nalbuphine free base, prodrug, salt, or mixture thereof equivalent to 1 mg to 50 mg, 1 mg to 45 mg, 1 mg to 40 mg, 1 mg to 35 mg, 1 mg to 30 mg, 1 mg to 25 mg, 1 mg to 20 mg, 1 mg to 15 mg, 1 mg to 10 mg, 1 mg to 9 mg, 1 mg to 8 mg, 2 mg to 8 mg, 3 mg to 8 mg, 1 mg to 7 mg, 2 mg to 7 mg, 3 mg to 7 mg, 4 mg to 7 mg, 1 mg to 6 mg, 2 mg to 6 mg, 3 mg to 6 mg, 1 mg to 5 mg, 2 mg to 5 mg, 1 mg to 4 mg, 1 mg to 3 mg, 1 mg to 2 mg nalbuphine hydrochloride salt administered intravenously.

In certain embodiments of the invention in which the pharmaceutical composition is to be administered sublingually (or other oral cavity administration), the amount of nalbuphine hydrochloride salt is preferably 1 to 60 times greater, 1 to 50 times greater, 1 to 45 times greater, 1 to 40 times greater, 5 to 50 times greater, 5 to 40 times greater, 5 to 35 times greater, 10 to 40 times greater, 15 to 40 times greater, 10 to 30 times greater, 15 to 30 times greater, 1 to 30 times greater, 1 to 20 times greater, 1 to 15 times greater, or 1 to 9 times greater by weight, than the amount of the opioid antagonist hydrochloride salt, preferably naloxone hydrochloride salt. Non-limiting examples of pharmaceutical compositions for sublingual administration are 8 mg nalbuphine hydrochloride salt with 0.4 mg naloxone hydrochloride salt (i.e., 20 times greater, by weight, nalbuphine hydrochloride salt than the naloxone hydrochloride salt), 15 mg nalbuphine hydrochloride salt with 3 mg naloxone hydrochloride salt (i.e., 5 times greater, by weight, nalbuphine hydrochloride salt than the naloxone hydrochloride salt), and 30 mg nalbuphine hydrochloride salt with 4 mg naloxone hydrochloride salt (i.e., 7.5 times greater, by weight, nalbuphine hydrochloride salt than the naloxone hydrochloride salt).

Alternatively, in certain embodiments of the present invention, the pharmaceutical composition for sublingual administration (or other oral cavity administration) comprises 5 mg to 65 mg, 5 mg to 60 mg, 5 mg to 55 mg, 5 mg to 50 mg, 5 mg to 40 mg, 5 mg to 35 mg, 6 mg to SS mg, 6 mg to 50 mg, 6 mg to 45 mg, 6 mg to 40 mg, 6 mg to 30 mg, 7 mg to 40 mg, 7 mg to 35 mg, or 7.5 to 30 mg of nalbuphine hydrochloride salt. In the preferred embodiment of the invention in which nalbuphine is the opioid agonist in the composition, naloxone hydrochloride salt is the preferred opioid antagonist. More preferably, the pharmaceutical composition intended for sublingual administration comprises 0.1 mg to 10 mg, 0.1 mg to 9 mg, 0.1 mg to 8 mg, 0.1 mg to 7 mg, 0.1 mg to 6 mg, 0.2 mg to 10 mg, 0.2 mg to 9 mg, 0.2 mg to 8 mg, 0.2 mg to 7 mg, 0.2 mg to 6 mg, 0.2 mg to 5 mg, 0.3 mg to 10 mg, 0.3 mg to 9 mg, 0.3 mg to 7 mg, 0.3 mg to 6 mg, 0.3 mg to 5 mg, 0.3 mg to 4 mg, 0.4 mg to 4 mg, 0.5 mg to 4 mg, 0.4 mg to 3.5 mg, 0.5 mg to 3.5 mg, 0.6 mg to 3.5 mg, 0.4 mg to 3 mg, 0.5 mg to 3 mg, 0.4 mg to 2.8 mg, 0.6 mg to 2.7 mg, 0.4 mg to 2.5 mg, 0.5 mg to 2.2 mg, 0.4 mg to 2 mg, 0.6 mg to 2 mg, 0.4 mg to 1.5 mg, or 0.4 mg to 1 mg naloxone hydrochloride salt. Non-limiting examples of the present invention are methods comprising administration of 5 mg, 5.5 mg, 6 mg, 6.5 mg, 7 mg, 7.5 mg, 8 mg, 8.5 mg, 9 mg, 9.5 mg 10 mg, 15 mg, 20 mg, 30 mg, 35 mg, 40 mg, 45 mg or 50 mg nalbuphine hydrochloride salt with naloxone hydrochloride salt. For example, in specific embodiments of the present invention, the method comprises sublingual administration of 0.4 mg of naloxone hydrochloride salt with 5 mg of nalbuphine hydrochloride salt, 0.4 mg of naloxone hydrochloride salt with 6 mg of nalbuphine hydrochloride salt, 0.4 mg of naloxone hydrochloride salt with 7 mg of nalbuphine hydrochloride salt, 0.4 mg of naloxone hydrochloride salt with 8 mg of nalbuphine hydrochloride salt, 0.4 mg of naloxone hydrochloride salt with 9 mg of nalbuphine hydrochloride salt, 0.4 mg of naloxone hydrochloride salt with 10 mg of nalbuphine hydrochloride salt, 0.4 mg of naloxone hydrochloride salt with 15 mg of nalbuphine hydrochloride salt, 0.4 mg of naloxone hydrochloride salt with 20 mg of nalbuphine hydrochloride salt, 0.4 mg of naloxone hydrochloride salt with 25 mg of nalbuphine hydrochloride salt, and 0.4 mg of naloxone hydrochloride salt with 30 mg of nalbuphine hydrochloride salt.

The present invention further encompasses pharmaceutical compositions comprising equivalent amounts of nalbuphine free base, non-hydrochloride salt, prodrug or mixture thereof to nalbuphine hydrochloride salt and equivalent amounts of the opioid antagonist free base, non-hydrochloride salt, prodrug, or mixture thereof to the opioid hydrochloride salt, or that releases the same amount of nalbuphine and antagonist into the site over a period of 15 minutes, 30 minutes, 1 hour, 2 hours, 4 hours, 6 hours, 8 hours, 10 hours, 12 hours, or 24 hours even if the same blood concentration is not achieved, provided that the when the opioid antagonist is naloxone free base, the amount of nalbuphine free base is not 5 mg and the amount of naloxone free base is not 0.4 mg.

In another preferred embodiment of the present invention, the pharmaceutical composition for administration other than by intravenous administration or oral administration for gastro-intestinal uptake comprises pentazocine, a prodrug, a pharmaceutically acceptable salt, or mixture thereof as the opioid agonist and an opioid antagonist in amounts equivalent to intravenous administration of the opioid antagonist hydrochloride salt and 18 to 120 times greater, 25 to 120 times greater, 18 to 110 times greater, 25 to 110 times greater. 18 to 100 times greater, 25 to 100 times greater, 18 to 95 times greater, 25 to 90 times greater, 30 to 90 times greater, 35 to 90 times greater, 18 to 85 times greater, 20 to 80 times greater, 20 to 60 times greater, 20 to 50 times greater, 25 to 55 times greater, 35 to 80 times greater, 20 to 75 times greater, 25 to 70 times greater, 40 to 100 times greater, 50 to 100 times greater, 55 to 95 times greater, 45 to 90 times greater, 40 to 70 times greater, 18 to 50 times greater, 18 to 40 times greater, 18 to 35 times greater, or 18 to 30 times greater, by weight, pentazocine hydrochloride salt than the opioid antagonist hydrochloride salt. Alternatively, the pharmaceutical composition comprises pentazocine, a prodrug, a pharmaceutically acceptable salt, or mixture thereof in amounts equivalent to 3 mg to 50 mg, 4 mg to 50 mg, 5 mg to 50 mg, 6 mg to 50 mg, 7 mg to 50 mg, 3 mg to 45 mg, 5 mg to 45 mg, 10 mg to 45 mg, 15 mg to 45 mg, 5 mg to 40 mg, 10 mg to 40 mg, 3 mg to 35 mg, 4 mg to 35 mg, 5 mg to 35 mg, 10 mg to 35 mg, 3 mg to 30 mg, 4 mg to 30 mg, 5 mg to 30 mg, 3 mg to 25 mg, 4 mg to 25 mg, 3 mg to 20 mg, 4 mg to 20 mg, 5 mg to 25 mg, 10 mg to 25 mg, 15 mg to 30 mg, 15 mg to 25 mg, 10 mg to 20 mg, and 10 mg to 15 mg pentazocine hydrochloride salt administered intravenously.

If the method of administration is sublingual, certain embodiments of the invention comprise of an opioid antagonist hydrochloride salt, preferably naloxone hydrochloride salt and 7.5 to 50 times greater, 7.5 to 45 times greater, 10 to 50 times greater, 10 to 40 times greater, 15 to 50 times greater, 15 to 45 times greater, 7.5 to 30 times greater, 7.5 to 25 times greater, 7.5 to 20 times greater, 10 to 30 times greater, or 7.5 to 15 times greater, by weight, pentazocine hydrochloride salt than the amount of opioid antagonist hydrochloride salt. Alternatively, in certain embodiments of the invention, the amount of pentazocine hydrochloride salt in the sublingual composition is 30 mg to 100 mg, 30 to 90 mg, 40 mg to 100 mg, 30 mg to 85 mg, 40 mg to 85 mg, 30 mg to 70 mg, 40 mg to 70 mg, 30 mg to 60 mg, 40 mg to 60 mg, 30 mg to 50 mg, 50 mg to 80, or 30 mg to 45 mg. In preferred embodiments of the invention for sublingual administration comprising pentazocine hydrochloride salt as the κ-opioid agonist, naloxone hydrochloride salt is the preferred opioid antagonist hydrochloride salt in amounts of 1 mg to 10 mg, 1 mg to 9 mg, 1 mg to 8 mg, 1 mg to 7 mg, 1 mg to 6 mg, 1 mg to 5 mg, 1 mg to 4 mg, 2 mg to 10 mg, 2 mg to 9 mg, 2 mg to 8 mg, 2 mg to 7 mg, 2 mg to 6 mg, 2 mg to 5 mg, 2 mg to 4 mg, 2 mg to 3.8 mg, 2 mg to 3.6 mg, 2 mg to 3.4 mg, 2 mg to 3.4 mg, 2.2 mg to 3.5 mg, 2 mg to 3.2 mg, 3 mg to 4 mg, or 2 mg to 3 mg. Non-limiting examples of the present invention are pharmaceutical compositions for sublingual administration comprising 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 50 mg, 55 mg, 60 mg, 65 mg, 70 mg, 75 mg, 80 mg, 85 mg, 90 mg, 95 mg or 100 mg pentazocine hydrochloride salt with 2 mg, 2.1 mg, 2.2 mg, 2.3 mg, 2.4 mg, 2.5 mg, 2.6 mg, 2.7 mg, 2.8 mg, 2.9 g, 3 mg, 3.1 mg, 3.2 mg, 3.3 mg, 3.4 mg, 3.5 mg, 3.6 mg, 3.7 mg, 3.8 mg, 3.9 mg, or 4.0 mg naloxone hydrochloride salt.

The present invention further encompasses pharmaceutical compositions comprising equivalent amounts of pentazocine free base, non-hydrochloride salt, prodrug or mixture thereof to pentazocine hydrochloride salt and equivalent amounts of the opioid antagonist free base, non-hydrochloride salt, prodrug, or mixture thereof to the opioid hydrochloride salt, or that releases the same amount of pentazocine and antagonist into the site over a period of 15 minutes, 30 minutes, 1 hour, 2 hours, 4 hours, 6 hours, 8 hours, 10 hours, 12 hours, or 24 hours even if the same blood concentration is not achieved, provided that if the pharmaceutical composition is for oral administration with gastro-intestinal uptake, the composition does not comprise 50 mg pentazocine hydrochloride salt and 0.5 mg naloxone hydrochloride salt.

In certain preferred embodiments, the pharmaceutical composition for administration other than intravenous administration comprises butorphanol, a prodrug, a pharmaceutically acceptable salt, or mixture thereof as the κ-opioid receptor agonist and an opioid antagonist, pharmaceutically acceptable salt, prodrug, or mixture thereof in amount equivalent to the opioid antagonist hydrochloride salt and 0.3 to 10 times greater, 0.3 to 9 times greater, 0.3 to 8 times greater, 0.5 to 10 times greater, 0.3 to 7 times greater, 0.5 to 7 times greater, 0.3 to 6 times greater. 0.5 to 6 times greater, 0.3 to 5 times greater, 0.5 to 5 times greater, 0.3 to 4 times greater, 0.5 to 4 times greater, 0.3 to 3 times greater, or 0.5 to 3 times greater, by weight, butorphanol tartrate salt than the opioid antagonist hydrochloride salt, preferably naloxone hydrochloride salt, administered intravenously. Alternatively, the amount of butorphanol, a prodrug, a pharmaceutically acceptable salt, or mixture thereof in the composition is equivalent to 0.2 mg to 2 mg, 0.2 mg to 1.9 mg, 0.2 mg to 1.8 mg, 0.2 mg to 1.7 mg, 0.25 mg to 1.9 mg, 0.25 mg to 1.8 mg, 0.25 mg to 1.75 mg, 0.25 mg to 1.5 mg, 0.25 mg to 1 mg, or 0.2 mg to 1 mg butorphanol tartrate salt administered intravenously.

If the pharmaceutical composition is intended to be administered sublingually, the amount of butorphanol tartrate salt is preferably 0.1 to 60 times greater, 0.1 to 50 times greater, 0.1 to 45 times greater, 0.3 to 40 times greater, or 0.5 to 30 greater, 10 to 60 times greater, 20 to 50 times greater, or 10 to 30 times greater, by weight, than the amount of opioid antagonist hydrochloride salt, preferably naloxone hydrochloride salt. Non-limiting examples of pharmaceutical compositions for sublingual administration are 0.5 mg butorphanol tartrate salt with 0.25 mg naloxone hydrochloride salt (i.e., 2 times greater, by weight, butorphanol tartrate salt than naloxone hydrochloride salt), 2 mg butorphanol tartrate salt with 0.2 mg naloxone hydrochloride salt (i.e., 10 times greater, by weight, butorphanol tartrate salt than naloxone hydrochloride salt), and 6 mg butorphanol tartrate salt with 0.3 mg naloxone hydrochloride salt (i.e., 20 times greater, by weight, butorphanol tartrate salt naloxone hydrochloride salt). Alternatively, the pharmaceutical composition for sublingual administration comprises 0.1 mg to 10 mg, 0.1 mg to 9 mg, 0.1 mg to 8 mg, 0.1 mg to 7 mg, 0.1 mg to 6 mg, 0.2 mg to 9 mg, 0.2 mg to 8 mg, 0.2 mg to 7 mg, 0.2 mg to 6 mg, 0.2 mg to 5.5 mg, 0.3 to 6.5 mg, 0.4 mg to 7 mg, or 0.5 mg to 6 mg of butorphanol tartrate salt as the κ-opioid agonist.

If the κ-opioid agonist in the pharmaceutical compositions for sublingual administration is butorphanol tartrate salt, then, preferably, the opioid antagonist is naloxone hydrochloride salt in amounts of 0.1 to 4 mg, 0.1 mg to 3.5 mg, 0.1 mg to 3 mg, 0.1 to 2.5 mg, 0.1 mg to 2 mg, 0.1 mg to 1 mg, 0.3 mg to 0.8 mg, or 0.1 mg to 0.8 mg. Non-limiting examples of the present invention are methods comprising administration of 0.1 mg, 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, or 7 mg butorphanol tartrate salt with 0.1 mg, 0.2 mg, 0.3 mg, 0.4 mg, 0.5 mg, 0.6 mg, 0.7 mg, or 0.85 mg naloxone hydrochloride salt. The present invention further encompasses pharmaceutical compositions comprising equivalent amounts of butorphanol free base, non-tartrate salt, prodrug or mixture thereof to butorphanol tartrate salt and equivalent amounts of the opioid antagonist free base, non-hydrochloride salt, prodrug, or mixture thereof to the opioid hydrochloride salt, or that releases the same amount of butorphanol and antagonist into the site over a period of 15 minutes, 30 minutes, 1 hour, 2 hours, 4 hours, 6 hours, 8 hours, 10 hours, 12 hours, or 24 hours even if the same blood concentration is not achieved.

In a preferred form of the invention, the combination of the κ-opioid agonist, preferably nalbuphine, for example as the hydrochloride salt, and opioid antagonist, preferably naloxone, for example as the hydrochloride salt, is administered to the patient or subject (preferably a human) by mucosal administration, particularly by intranasal or pulmonary administration. In such administration a composition containing the two ingredients suitable for such administration, is used, as described hereinafter. In such administration, the amounts of the opioid agonist and antagonist, and the ratio of the one to the other, are as described above. Particularly good results are generally obtained by administration of the combination wherein the weight ratio of nalbuphine component to naloxone component is from about 10:1 to about 15:1, most preferably about 12.5:1 and where the amount of nalbuphine (as the hydrochloride salt) is 5 mg and the amount of naloxone (as the hydrochloride salt) is about 0.4 mg. Similarly, the two may be administered at one-quarter of that dosage (e.g., 1.25 mg nalbuphine hydrochloride and 0.1 mg naloxone hydrochloride), at one-half the dosage (e.g., 2.5 mg nalbuphine hydrochloride and 0.2 mg naloxone hydrochloride) at twice (e.g., 10 mg nalbuphine hydrochloride and 0.8 mg naloxone hydrochloride) or at four times that dosage (e.g., 20 mg hydrochloride and 1.6 mg naloxone hydrochloride). Such dosage variations may be used, for example, for patients with lesser body mass or less pain the dose may be lower and for patients with greater body mass or more severe pain the dose may be larger.

### Administration and Formulations

Methods of administering κ-opioid receptor agonist and opioid antagonists are well known in the art. Methods of administration include, but are not limited to, parenteral administration (e.g., intradermal, intramuscular, intra peritoneal, intravenous, and subcutaneous) and mucosal (e.g., intranasal, oral, and rectal routes). In preferred embodiments of the present invention, the κ-opioid receptor agonist and opioid antagonist and pharmaceutical compositions thereof are administered intravenously or (most preferably) mucosally, including, but not limited to nasal, sublingual (or other oral cavity administration), pulmonary (i.e., inhaled into the lungs, such as by an inhaler or nebulizer), and rectal administration. The κ-opioid receptor agonist and opioid antagonist and pharmaceutical compositions thereof may be administered alone or together with other biologically active agents, e.g., as described in this section. Administration can be systemic or local.

The κ-opioid agonist and opioid antagonist of the invention may be administered by different methods, although, preferably, the agonist and antagonist are administered by the same method, and most preferably, by mucosal administration, by intravenous administration, or sublingual administration (or other oral cavity administration not for significant gastro-intestinal uptake). For example, the opioid agonist and opioid antagonist may be administered intravenously or mucosally together, or the opioid agonist administered intramuscularly and the opioid antagonist administered intravenously. Other routes of administration include intramuscular, subcutaneous and intrathecal injection. For parenteral administration (intravenous, intramuscular, subcutaneous, or intrathecal injection), the opioid agonist and antagonist of the invention are preferably in a sterile aqueous solution that may also contain other dissolved substances such as, but not limited to, preservatives, stabilizers, and pH adjusting agents.

The κ-opioid receptor agonist and opioid antagonist are preferably administered simultaneously, but can be administered sequentially in any order. Sequential administration is carried out within a time period such that the opioid antagonist modulates the effects (i.e., analgesia and/or adverse side effects) of the κ-opioid agonist. Preferably, the opioid agonist and antagonist are administered within 10 to 12 hours, 6 to 8 hours, 3 to 6 hours, 3 hours, 2 hours, 1 hour, 15 to 45 minutes, or 10 to 15 minutes, and most preferably, substantially at the same time. The compositions can also be formulated for and administered according to patient controlled analgesia methods known in the art.

The invention encompasses pharmaceutical compositions comprising an amount of a κ-Opioid receptor agonist and opioid antagonist that produces greater analgesia than the administration of either the agonist or antagonist alone, together with a suitable amount of carrier so as to provide the form for proper administration to the patient. The formulation of the invention should suit the mode of administration. The amount of the compositions of the invention which will be effective in treating, managing, or ameliorating pain can be determined by standard clinical techniques. The precise dose to be employed in the formulation will depend on the route of administration, the intensity of pain the subject is experiencing, gender of the subject, and the size and weight of the subject, and should be decided according to the judgment of the practitioner and each patient's circumstances.

The pharmaceutical compositions of the invention are formulated to be compatible with their intended route of administration. Examples of routes of administration include, but are not limited to, parenteral, e.g., intravenous, intradermal, subcutaneous, and mucosal, e.g., nasal, sublingual, pulmonary, or rectal. In a specific embodiment, the composition is formulated in accordance with routine procedures as a pharmaceutical composition adapted for mucosal, intravenous or sublingual administration to human beings.

In one preferred embodiment, a pharmaceutical composition is formulated in accordance with routine procedures for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. In specific embodiments, the dosage form for intravenous administration comprises 1 mg, 1.25 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 15 mg, 20 mg, 25 mg, 30 mg, 40 mg, or 50 mg of nalbuphine hydrochloride salt with 0.1 mg, 0.15 mg, 0.2 mg, 0.2 mg, 0.25 mg, 0.3 mg, 0.35 mg, 0.4 mg, 0.5 mg, 0.6 mg, 0.7 mg, 0.8 mg, 1 mg, 1.6 mg, 2 mg, 2.4 mg, 3 mg, 4 mg, 5 mg, 6 mg, 7 mg and 8 mg of naloxone hydrochloride salt.

If the compositions of the invention are to be administered orally, preferably sublingually, and, in certain embodiments, not for gastro-intestinal uptake, the compositions can be formulated orally in the form of, e.g., tablets, capsules, cachets, gelcaps, solutions, suspensions and the like. Tablets or capsules can be prepared by conventional means with pharmaceutically acceptable excipients such as binding agents (e.g., pregelatinzed maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well-known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated for slow release, controlled release or sustained release of a prophylactic or therapeutic agent(s).

The compositions of the invention may also be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The present invention also includes pharmaceutical compositions for the treatment of pain comprising of a κ-opioid receptor agonist and an opioid antagonist that is to be administered by a method other than intravenous administration. The composition, shape, and type of dosage forms of the invention will vary depending on their use and method of administration. The differences of the specific dosage forms encompassed by this invention are readily apparent to those skilled in the art. See, e.g., Remington 's Pharmaceutical Sciences, 18^{th} ed., Mack Publishing, Easton, Pa. (1990).

If the compositions of the invention are to be administered mucosally through the nasal cavity, the compositions can be formulated in an aerosol form, spray, mist or in the form of drops. In particular, the compositions of the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compositions of the invention may also be formulated in rectal compositions such as suppositories or retention enemas, e.g., containing conventional suppository bases such as cocoa butter or other glycerides.

The compositions of the invention may also be formulated for transdermal administration. For transdermal administration, the active compounds are formulated into ointments, salves, gels, or creams as generally known in the art. Pharmaceutical compositions adapted for transdermal administration can be provided as discrete patches intended to remain in intimate contact with the epidermis for a prolonged period of time. If the compositions of the invention are to be administered topically, the compositions can be formulated in the form of, e.g., an ointment, cream, transdermal patch, lotion, gel, spray, aerosol, solution, emulsion, or other form well-known to one of skill in the art. For nonsprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as Freon), or in a squeeze bottle. Moisturizers or humectants can also be added to pharmaceutical compositions and dosage forms if desired. Examples of such additional ingredients are well-known in the art.

In addition to the formulations described previously, the compositions of the invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt. Compositions of the present invention may also be prepared in unit dosage forms or in concentrated forms that may be diluted before administration.

A preferred method of administration of the compositions of this invention is mucosal administration, particularly intranasal administration or administration by inhalation (pulmonary administration). Pulmonary drug delivery can be achieved by several different approaches, including liquid nebulizers, aerosol-based metered dose inhalers (MDIs), and dry powder dispersion devices. Compositions for use in administrations of this type are typically dry powders or aerosols. For administration of aerosols, which is the preferred method of administration of this invention, the compositions are delivered by inhalers, some types of which are described below.

Dry powders contain, in addition to the active ingredient, a carrier, an absorption enhancer, and optionally other ingredients. The carrier is, for example, a mono-, di- or polysaccharide, a sugar alcohol or another polyol. Suitable carriers include lactose, glucose, raffinose, melezitose, lactitol, maltitol, trehalose, sucrose, mannitol; and starch. Lactose is particularly preferred, especially in the form of its monohydrate. Also included are absorption enhancers such as polypeptides, surfactants, alkyl glycosides, amine salts of fatty acids or phospholipids. The ingredients of the formulation typically must be in a finely divided form, i.e. their volume median diameter should generally be from about 30 to about 200 microns, as measured by a laser diffraction instrument or a coulter counter. The desired particle size may be produced using methods known in the art, e.g. milling, micronization or direct precipitation.

The intranasal route of administration provides numerous advantages over intravenous and intramuscular injections. For instance, one advantage of intranasal administration is convenience. An injectable system requires sterilization of the hypodermic syringe and in the institutional setting, leads to concerns among medical personnel about the risk of contracting disease by being accidentally stuck by a contaminated needle. Strict requirements for the safe disposal of the used needle and syringe must also be imposed in the institutional setting. In contrast, intranasal administration requires little time on the part of the patient and the attending medical personnel, and is far less burdensome on the institution than injectables.

A second important advantage of intranasal administration over IM and IV is patient acceptance of the drug delivery system. Intranasal administration is perceived as noninvasive, is not accompanied by pain, has no significant after-effects and produces the gratification of prompt relief in the patient exhibiting the symptom. This is of particular advantage when the patient is a child. Another important consideration is that the patient may be able to self-administer the prescribed dosage(s) of nasal spray.

For intranasal administration the compositions of this invention may be formulated as liquids or as solids. Such compositions may contain one or more adjuvants, agents for enhancing absorption of the active ingredients by permeation across the nasal membrane, and (for liquid compositions) an aqueous diluent, for instance water. Alternatively, the diluent may comprise an aqueous buffer such as phosphate buffer. The composition may further optionally include one or more polyhydric alcohols and one or more preservative agents such as, for example, gentamicin, bacitracin (0.005%), or cresol. The compositions may be administered to the nasal cavity in the form of a spray by using an atomizer, nebulizer, sprayer, dropper or other device which insures contact of the solution with the nasal mucous membrane. The device may be a simple one such as a simple nasal sprayer that may be used by the patient, or may be a more elaborate instrument for more accurate dispensing of the compositions, that may be used in a physician's office or a medical facility.

Nasal powder compositions can be made by mixing the active agent and the excipient, both possessing the desired particle size. Firstly, a solution of the active agent and the cyclodextrin excipients made, followed by precipitation, filtration and pulverization. It is also possible to remove the solvent by freeze drying, followed by pulverization of the powder in the desired particle size by using conventional techniques, known from the pharmaceutical literature. The final step is size classification for instance by sieving, to get particles that are preferably between 30 and 200 microns in diameter. Powders can be administered using a nasal insufflator, or they may be placed in a capsule set in an inhalation or insufflation device. A needle is penetrated through the capsule to make pores at the top and the bottom of the capsule and air is sent to blow out the powder particles. Powder formulation can also be administered in a jet-spray of an inert gas or suspended in liquid organic fluids.

In a specific embodiment, the pharmaceutical composition can be delivered in a controlled or sustained release system. In one embodiment, a pump may be used to achieve a controlled or sustained release (see Langer, Science, 249:1527-1533 (1990); Sefton, 1987, CRC Crit. Ref. Biomed. Eng. 14:10; Buschwald et al., 1980, Surgery 88:507; Saudek et al., 1989 N. Engl. J. Med. 321:574). In another embodiment, polymeric materials can be used to achieve controlled or sustained release of the κ-opioid receptor agonist and/or opioid antagonist (see e.g., Medical Applications of Controlled Release, Langer and Wise (eds.), CRC Pres., Boca Raton, Fla. 1974); Controlled Drug Bioavailability, Drug Product Design and Performance, Smolen and Ball (eds.), Wiley, New York (1984); Ranger and Peppas, 1983, J. Macromol. Sci. Rev. Macrol. Chem. 23:61; see also Levy et al., 1985 Science 228:190; During et al., 1989, Ann. Neurol. 25:351; Howard et al., 1989, J. Neurosurg. 71:105; U.S. Pat. No. 5,679,377; U.S. Pat. No. 5,916,597, U.S. Pat. No. 5,912,015; U.S. Pat. No. 5,989,463; U.S. Pat. No. 5,128,326; PCT Publication No. WO 99/12154; and PCT Publication No. WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polyactides (PLA), poly(lactideco-glycolides)(PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity to the therapeutic target, thus requiring only a fraction of the systematic dose (see, e.g., Goodson, in Medical Applications of Controlled Release, supra, vol. 2, pp. 115-138 (1984)).

In a specific embodiment, the pharmaceutical composition is formulated for differential release so that either the κ-opioid agonist or the opioid antagonist is released first and then the antagonist or the agonist, respectively is released 10 to 12 hours, 6 to 8 hours, 3 to 6 hours, 3 hours, 2 hours, 1 hour, 15 to 45 minutes, or 10 to 15 minutes after the first release, or according to the practitioner's judgment. In such a controlled or sustained release formulation, the amount of the κ-opioid agonist and opioid antagonist delivered over time may be equivalent to an amount delivered at one time by a different type of formulation for the same mode of administration.

### Methods of Treatment

The invention provides methods of treating, managing, or ameliorating pain by administration of a κ-opioid receptor agonist with an opioid antagonist such that the analgesia achieved by administration is greater than with administration of either the κ-opioid receptor agonist or the opioid antagonist alone (or, in certain embodiments, is greater than the additive analgesic effect of the agonist and antagonist administered alone). The subject is preferably a mammal, such as a non-primate (e.g., cows, pigs, horses, cats, dogs, rats, etc.) or a primate (e.g., monkey, such as a cynomolgous monkey, and human). In a preferred embodiment, the subject is a human. The subject can be male, female, adult, adolescent, or infant. The specific embodiments, the present invention encompasses methods and compositions for the treatment of pain in children, adolescents, and the elderly. In specific embodiments, the dose of opioid agonist and antagonist administered in accordance with the present invention is reduced based on the weight of the subject receiving treatment. In another preferred embodiment, the subject is not or preferably, has not been, addicted to opioids, particularly μ opioids, such as, but not limited to, morphine, codeine, or methadone, or κ-opioids, such as, but not limited to pentazocine.

Methods for assaying for pain are well known in the art. Certain methods for assaying for pain include the use of a visual analog scale (VAS). This is a subjective measurement of pain, in which persons participating in the study are requested to indicate a level of pain at a certain time on a 10-cm line. The patient makes an indication on the line at a value from 0 to 10 indicating the level of pain felt at that time (where 0 indicates no pain and 10 indicates the worst pain imaginable to the patient). Patients rated pain on the VAS at 20 minute intervals, both before and after administration of the active agents according to this invention. Tabulations were then made indicating increase or decrease in the pain level with time, with a negative value indicating a decrease in pain experienced by the patient and a positive value, an increase in such pain.

In accordance to this invention, the combination of κ-opioid receptor agonists and opioid antagonists can be used to treat or prevent acute or chronic and regional or generalized pain. For example, the combinations can be used for, but are not limited to, treating or preventing inflammatory pain, neuropathic pain, acute chronic pain, cancer pain, labor pain, myocardial infarction pain, pancreatic pain, colic pain, headache pain, pain associated with intensive care, pain associated with thalamic syndrome, and regional pain syndromes, e.g., reflex sympathetic dystrophy, sympathetic maintained pain syndrome and casalgia, and generalized pain syndromes, e.g., fibromyalgia, irritable bowel syndrome, temperal-mandibulla disorders, syndrome X, and migraine headaches. The present invention is particularly useful in treating, managing, or ameliorating post-operative pain.

Certain embodiments of the invention provide methods treating and lessening pain in subjects experiencing inflammatory pain. The inflammatory pain may be acute or chronic and can be due to any condition characterized by inflammation, including, but not limited to, sunburn, rheumatoid arthritis, osteoarthritis, colitis, carditis, dermatitis, myositis, neuritis, and collagen vascular diseases. The method of the present invention comprising administration of the combination of κ-opioid receptor agonists and opioid antagonists in amounts taught by this invention reduces both the acute pain and chronic hyperalgesia that the subject suffers.

In another embodiment, the invention provides methods for treating neuropathic pain in a subject. Such subjects can have neuropathy classified as a radiculopathy, mononeuropathy, mononeuropathy multiplex, polyneuropathy, or plexopathy. Diseases in these classes can be caused by a variety of nerve-damaging conditions or procedures, including, but not limited to, trauma, stroke, demyelinating diseases, abscess, surgery, amputation, inflammatory diseases of the nerves, causalgia, diabetes, collagen vascular diseases, trigeminal neuralgia, rheumatoid arthritis, toxins, chronic alcoholism, complex regional pain syndrome type I, complex regional pain syndrome type II, AIDS and other viral infections, including herpes, cancer (which can cause direct or remote (e.g., paraneoplastic) nerve damage), anti-viral therapies (e.g., AIDS or hepatitis therapies (such as nucleoside analogs and protease inhibitors)), and cancer treatment (e.g., vinca, alkaloids, taxanes, cyclophosphamide, and melphalan). Nerve damage causing hyperalgesia can be in peripheral or CNS nerves or both.

The invention encompasses methods of treating, managing, or ameliorating pain by administration of an amount of a κ-opioid receptor agonists and an opioid antagonist to produce greater analgesia than administration of either the agonist or antagonist alone. The precise dosage of the κ-opioid agonist and opioid antagonist will depend on the route of administration, the intensity of pain of the subject is experiencing, and the size and weight of the subject, and should be decided according to the judgment of the practitioner and each patient's circumstances. The κ-opioid agonist and opioid antagonist can be administered to subjects in dosages suitable for continuous administration, administration once daily, administration twice daily, administration three times a day, administration four times a day, administration six times a daily, administration every other day, and in other dosage regimens that the practitioner determines to be appropriate. The dosages can also be self-administered by the patient as patient controlled analgesic therapy.

In particular embodiments, the invention provides for methods of treating, managing, or ameliorating pain by intravenous administration of an amount of a κ-opioid receptor agonist and an amount of an opioid antagonist that results in greater analgesia than that which results from administration of either the agonist or the antagonist alone. The invention encompasses methods of intravenous administration or mucosal administration (e.g., nasal or pulmonary administration) of 0.02 mg to 8 mg, 0.02 to 7 mg, 0.02 mg to 6 mg, 0.02 to 5 mg, 0.02 to 4 mg, 0.02 to 3 mg, 0.02 to 2 mg, 0.02 to 2 mg, 0.1 mg to 8 mg, 0.1 mg to 7 mg, 0.1 mg to 6 mg, 0.1 mg to 5 mg, 0.1 mg to 4 mg, 0.1 mg to 3 mg, 0.1 mg to 2 mg, 0.1 mg to 1 mg, 0.2 mg to 8 mg, 0.2 mg to 7 mg, 0.2 mg to 6 mg, 0.2 mg to 4 mg, 0.2 mg to 3 mg, 0.2 mg to 2 mg, 0.2 mg to 1 mg, 0.4 mg to 8 mg, 0.4 to 7 mg, 0.4 to 6 mg, 0.4 mg to 5 mg, 0.4 mg to 4 mg, 0.4 mg to 3 mg, 0.4 mg to 2 mg, 0.4 mg to 1 mg, 0.5 mg to 8 mg, 0.5 mg to 6 mg, 0.5 mg to 5 mg, 0.5 mg to 4 mg, 0.5 mg to 3 mg, 0.5 mg to 3 mg, 0.5 to 2 mg, 0.5 mg to 1 mg, 1 mg to 8 mg, 1 mg to 6 mg, 1 mg to 5 mg, 1 mg to 4 mg, 1 mg to 3 mg, 1 mg to 2 mg, 5 mg to 8 mg, 4 mg to 7 mg, 3 mg to 5 mg, 0.02 mg to 1 mg, preferably 0.1 mg to 0.8 mg, 0.2 mg to 0.8 mg, 0.3 mg to 0.8 mg, 0.4 mg to 0.8 mg, 0.5 mg to 0.8 mg, 0.1 mg to 0.7 mg, 0.2 mg to 0.7 mg, 0.3 mg to 0.7 mg, 0.4 mg to 0.7 mg, 0.1 mg to 0.6 mg, 0.2 mg to 0.6 mg, 0.3 to 0.6 mg, 0.1 mg to 0.5 mg, 0.15 mg to 0.5 mg, 0.2 mg to 0.5 mg, 0.25 mg to 0.5 mg, 0.3 mg to 0.5 mg, 0.35 mg to 0.5 mg, 0.1 mg to 0.45 mg, 0.15 mg to 0.45 mg, 0.2 mg to 0.45 mg, 0.25 mg to 0.45 mg, 0.1 mg to 0.3 mg, 0.13 mg to 0.3 mg, 0.2 mg to 0.3 mg, 0.1 mg to 0.25 mg, 0.15 mg to 0.25 mg, 0.1 mg to 0.2 mg, 0.1 mg to 0.15 mg naloxone hydrochloride salt. Typical amounts of naloxone hydrochloride salt are 0.1 mg, 0.15 mg, 0.2 mg, 0.25 mg, 0.3 mg, 0.35 mg, 0.4 mg, 0.45 mg, 0.5 mg, 0.55 mg, 0.6 mg 0.7 mg, 0.75 mg, 0.8 mg, 1 mg, 1.6 mg, 2 mg, 2.4 mg, 3 mg, 4 mg, 5 mg, 6 mg, 7 mg and 8 mg naloxone hydrochloride salt.

The invention also encompasses administration of naloxone free base, non-hydrochloride salt, prodrug, or mixture thereof in equivalent amounts to intravenous administration of naloxone hydrochloride salt.

In general, the amount of κ-opioid agonist to be administered with the antagonist is in the range of 5% to 100% of the recommended analgesic dose (e.g., as provided in the Physician's Desk Reference or other commonly used reference). Preferably, the dose the κ-opioid agonist is 5% to 90%, 10% to 90%, 5% to 85%, 10% to 85%, 15% to 85%, 20% to 80%, 5% to 75%, 10% to 75%, 15% to 70%, 15% to 60%, 5% to 55%, 10% to 55%, 15% to 50%, 5% to 50%, 5% to 45%, 5% to 40%, 5% to 35%, 5% to 30%, 10% to 40%, 10% to 35%, 10% to 30%, 15% to 40%, 15% to 35%, 15% to 30%, 5% to 25%, 10% to 25%, 15% to 25%, 5% to 20%, and 5% to 15% of the recommended analgesic dose. However, as mentioned above, the amount administered may be a fraction or a multiple of the recommended dosage.

The precise dosages of the κ-opioid agonist and opioid antagonist will depend on the size and weight of the subject, the route of administration, and the intensity of pain the subject is experiencing, and should be decided according to the judgment of the practitioner and each patient's circumstances. For example, children and adolescents may be administered smaller amounts of the κ-opioid agonist and opioid antagonist compositions of the present invention than an adult and an obese adult may be administered larger dosages of the κ-opioid agonist and opioid antagonist composition than those dosages administered to a non-obese adult according to the methods of the present invention.

In a specific embodiment of the invention, the opioid agonist is nalbuphine free base, prodrug, or mixture thereof, but most preferably nalbuphine hydrochloride salt is administered. In specific embodiments, the amount of nalbuphine salt administered intravenously is 6.25 to 49 times greater, 6.25 to 40 times greater, 6.25 to 35 times greater, 6.25 to 30 times greater, 6.25 to 20 times greater, 8 to 35 times greater, 8 to 30 times greater, 8 to 25 times greater, 10 to 30 times greater, 10 to 20 times greater, 15 to 25 times greater, 20 to 25 times greater, 10 to 15 times greater, 9 to 15 times greater, 5 to 10 times greater, 30 to 49 times greater, 20 to 30 times greater, 15 to 20 times greater, 13 to 15 times greater, 11 to 13 times greater, 9 to 11 times greater, 7 to 9 times greater, or 6.25 to 7 times greater, by weight, than the opioid antagonist hydrochloride salt, preferably naloxone hydrochloride salt. In a specific embodiments, the opioid antagonist hydrochloride salt is preferably naloxone hydrochloride salt. As non-limiting examples, the method of the present invention comprises administration of 3 mg of nalbuphine hydrochloride salt with 0.4 mg naloxone hydrochloride salt (i.e., 7.5 times greater nalbuphine hydrochloride salt than the opioid antagonist hydrochloride salt); 1.25 mg nalbuphine hydrochloride salt with 0.1 mg naloxone hydrochloride salt, 2.5 mg nalbuphine hydrochloride salt with 0.2 mg naloxone hydrochloride salt, 5 mg nalbuphine hydrochloride salt with 0.4 mg naloxone hydrochloride salt, 10 mg nalbuphine hydrochloride salt with 0.8 mg naloxone hydrochloride salt, 20 mg nalbuphine hydrochloride salt with 1.6 mg naloxone hydrochloride salt, 25 mg nalbuphine hydrochloride salt with 2.0 mg naloxone hydrochloride salt, and 30 mg nalbuphine hydrochloride salt with 2.4 mg naloxone hydrochloride salt (i.e., 12.5 times greater, by weight, nalbuphine hydrochloride salt than naloxone hydrochloride salt); 5 mg nalbuphine hydrochloride salt with 0.2 mg naloxone hydrochloride salt and 10 mg nalbuphine hydrochloride salt with 0.4 mg naloxone hydrochloride salt (i.e., 25 times greater nalbuphine hydrochloride salt than the naloxone hydrochloride salt); 4.9 mg nalbuphine hydrochloride salt with 0.1 mg naloxone hydrochloride salt and 9.8 mg nalbuphine hydrochloride salt with 0.2 mg naloxone hydrochloride salt (i.e., 49 times greater, by weight, nalbuphine hydrochloride salt than naloxone hydrochloride salt).

In a specific embodiment, the weight of nalbuphine hydrochloride salt administered intravenously or mucosally is 1 mg to 50 mg, 1 mg to 45 mg, 1 mg to 40 mg, 1 mg to 35 mg, 1 mg to 30 mg, 1 mg to 25 mg, 1 mg to 20 mg, 1 mg to 15 mg, 1 mg to 10 mg, 1 mg to 9 mg, 1 mg to 8 mg, 2 mg to 8 mg, 3 mg to 8 mg, 1 mg to 7 mg, 2 mg to 7 mg, 3 mg to 7 mg, 4 mg to 7 mg, 1 mg to 6 mg, 2 mg to 6 mg, 3 mg to 6 mg, 1 mg to 5 mg, 2 mg to 5 mg, 1 mg to 4 mg, 1 mg to 3 mg, 1 mg to 2 mg. In certain preferred embodiments in which nalbuphine hydrochloride salt is administered as the opioid agonist, 0.02 mg to 8 mg, 0.02 to 7 mg, 0.02 mg to 6 mg, 0.02 to 5 mg, 0.02 to 4 mg, 0.02 to 3 mg, 0.02 to 2 mg, 0.02 to 2 mg, 0.1 mg to 8 mg, 0.1 mg to 7 mg, 0.1 mg to 6 mg, 0.1 mg to 5 mg, 0.1 mg to 4 mg, 0.1 mg to 3 mg, 0.1 mg to 2 mg, 0.1 mg to 1 mg, 0.2 mg to 8 mg, 0.2 mg to 7 mg, 0.2 mg to 6 mg, 0.2 mg to 4 mg, 0.2 mg to 3 mg, 0.2 mg to 2 mg, 0.2 mg to 1 mg, 0.4 mg to 8 mg, 0.4 to 7 mg, 0.4 to 6 mg, 0.4 mg to 5 mg, 0.4 mg to 4 mg, 0.4 mg to 3 mg, 0.4 mg to 2 mg, 0.4 mg to 1 mg, 0.5 mg to 8 mg, 0.5 mg to 6 mg, 0.5 mg to 5 mg, 0.5 mg to 4 mg, 0.5 mg to 3 mg, 0.5 mg to 3 mg, 0.5 to 2 mg, 0.5 mg to 1 mg, 1 mg to 8 mg, 1 mg to 6 mg, 1 mg to 5 mg, 1 mg to 4 mg, 1 mg to 3 mg, 1 mg to 2 mg, 5 mg to 8 mg, 4 mg to 7 mg, 3 mg to 5 mg, 0.02 mg to 1 mg, preferably 0.1 mg to 0.8 mg, 0.2 mg to 0.8 mg, 0.3 mg to 0.8 mg, 0.4 mg to 0.8 mg, 0.5 mg to 0.8 mg, 0.1 mg to 0.7 mg, 0.2 mg to 0.7 mg, 0.3 mg to 0.7 mg, 0.4 mg to 0.7 mg, 0.1 mg to 0.6 mg, 0.2 mg to 0.6 mg, 0.3 to 0.6 mg, 0.1 mg to 0.5 mg, 0.15 mg to 0.5 mg, 0.2 mg to 0.5 mg, 0.25 mg to 0.5 mg, 0.3 mg to 0.5 mg, 0.35 mg to 0.5 mg, 0.1 mg to 0.45 mg, 0.15 mg to 0.45 mg, 0.2 mg to 0.45 mg, 0.25 mg to 0.45 mg, 0.1 mg to 0.3 mg, 0.13 mg to 0.3 mg, 0.2 mg to 0.3 mg, 0.1 mg to 0.25 mg, 0.15 mg to 0.25 mg, 0.1 mg to 0.2 mg, 0.1 mg to 0.15 mg naloxone hydrochloride salt is administered as the opioid antagonist hydrochloride salt. Non-limiting examples of the method of the present invention comprise intravenous administration of 1 mg, 1.25 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 15 mg, mg, 25 mg, 30 mg, 40 mg, 50 mg, to 60 mg of nalbuphine hydrochloride salt with 0.1 mg, 0.15 mg, 0.2 mg, 0.2 mg, 0.25 mg, 0.3 mg, 0.35 mg, 0.4 mg, 0.5 mg, 0.6 mg, 0.7 mg, 0.8 mg, 1 mg, 1.6 mg, 2 mg, 2.4 mg, 3 mg, 4 mg, 5 mg, 6 mg, 7 mg and 8 mg of naloxone hydrochloride salt.

For example, specific embodiments of the invention comprise intravenous administration of 0.1 mg naloxone hydrochloride salt with 1.25 mg nalbuphine hydrochloride salt, 0.2 mg of naloxone hydrochloride salt with 1 mg nalbuphine hydrochloride salt, 0.2 mg of naloxone hydrochloride salt with 1.5 mg nalbuphine hydrochloride salt, 0.2 mg of naloxone hydrochloride salt with 2 mg nalbuphine hydrochloride salt, 0.2 mg of naloxone hydrochloride salt with 2.5 mg nalbuphine hydrochloride salt, 0.2 mg of naloxone hydrochloride salt with 3 mg nalbuphine hydrochloride salt, 0.2 mg of naloxone hydrochloride salt with 3.5 mg nalbuphine hydrochloride salt, 0.2 mg of naloxone hydrochloride salt with 4 mg nalbuphine hydrochloride salt, 0.2 mg of naloxone hydrochloride salt with 4.5 mg nalbuphine hydrochloride salt, 0.2 mg of naloxone hydrochloride salt with 5 mg nalbuphine hydrochloride salt, 0.2 mg of naloxone hydrochloride salt with 5.5 mg nalbuphine hydrochloride salt, 0.2 mg of naloxone hydrochloride salt with 6 mg nalbuphine hydrochloride salt, 0.2 mg of naloxone hydrochloride salt with 6.5 mg nalbuphine hydrochloride salt, 0.2 mg of naloxone hydrochloride salt with 7 mg nalbuphine hydrochloride salt 0.2 mg of naloxone hydrochloride salt with 7.5 mg nalbuphine hydrochloride salt, 0.2 mg of naloxone hydrochloride salt with 8 mg nalbuphine hydrochloride salt, 0.2 mg of naloxone hydrochloride salt with 8.5 mg nalbuphine hydrochloride salt, 0.2 mg of naloxone hydrochloride salt with 9 mg nalbuphine hydrochloride salt, 0.2 mg of naloxone hydrochloride salt with 9.5 mg nalbuphine hydrochloride salt, 0.2 mg of naloxone hydrochloride salt with 10 mg nalbuphine hydrochloride salt, 0.4 mg of naloxone hydrochloride salt with 1 mg nalbuphine hydrochloride salt, 0.4 mg of naloxone hydrochloride salt with 1.5 mg nalbuphine hydrochloride salt, 0.4 mg of naloxone hydrochloride salt with 2.0 mg nalbuphine hydrochloride salt, 0.4 mg of naloxone hydrochloride salt with 2.5 mg nalbuphine hydrochloride salt, 0.4 mg of naloxone hydrochloride salt with 3 mg nalbuphine hydrochloride salt, 0.4 mg of naloxone hydrochloride salt with 3.5 mg nalbuphine hydrochloride salt, 0.4 mg of naloxone hydrochloride salt with 4 mg nalbuphine hydrochloride salt, 0.4 mg of naloxone hydrochloride salt with 4.5 mg nalbuphine hydrochloride salt, 0.4 mg of naloxone hydrochloride salt with 5 mg nalbuphine hydrochloride salt, 0.4 mg of naloxone hydrochloride salt with 5.5 mg nalbuphine hydrochloride salt, 0.4 mg of naloxone hydrochloride salt with 6 mg nalbuphine hydrochloride salt, 0.4 mg of naloxone hydrochloride salt with 6.5 mg nalbuphine hydrochloride salt, 0.4 mg of naloxone hydrochloride salt with 7 mg nalbuphine hydrochloride salt, 0.4 mg of naloxone hydrochloride salt with 7.5 mg nalbuphine hydrochloride salt, 0.4 mg of naloxone hydrochloride salt with 8 mg nalbuphine hydrochloride salt, 0.4 mg of naloxone hydrochloride salt with 8.5 mg nalbuphine hydrochloride salt, 0.4 mg of naloxone hydrochloride salt with 9 mg nalbuphine hydrochloride salt, 0.4 mg of naloxone hydrochloride salt with 9.5 mg nalbuphine hydrochloride salt, 0.4 mg of naloxone hydrochloride salt with 10 mg nalbuphine hydrochloride salt, and 0.8 mg of naloxone hydrochloride salt with 10 mg nalbuphine hydrochloride salt. The present invention also encompasses methods comprising intravenous administration of nalbuphine free base, non-hydrochloride salt, prodrug, or mixture thereof in an amount equivalent to nalbuphine hydrochloride salt and/or administration of the opioid antagonist free base, non-hydrochloride salt, prodrug, or mixture thereof in an equivalent amount to the opioid antagonist hydrochloride salt, provided that 5 mg nalbuphine free base is not administered with 0.4 mg naloxone free base.

The present invention also encompass methods comprising administration of pentazocine free base, non-hydrochloride salt, prodrug, or mixture thereof in an equivalent amount to pentazocine hydrochloride salt and/or administration of the opioid antagonist free base, non-hydrochloride salt, prodrug, or mixture thereof in an equivalent amount to the opioid antagonist hydrochloride salt.

In another embodiment of the invention, pentazocine free base, prodrug, non-hydrochloride salt, most preferably in the hydrochloride salt, or mixture thereof is administered intravenously as the κ-opioid receptor agonist with the opioid antagonist to treat, manage, or ameliorate pain. In certain embodiments, the method of the invention comprises administration of an opioid antagonist hydrochloride salt, particularly naloxone hydrochloride salt, and 18 to 120 times greater, 25 to 120 times greater, 18 to 110 times greater, 25 to 110 times greater. 18 to 100 times greater, 25 to 100 times greater, 18 to 95 times greater, 25 to 90 times greater, 30 to 90 times greater, 35 to 90 times greater, 18 to 85 times greater, 20 to 80 times greater, 20 to 60 times greater, 20 to 50 times greater, 25 to 55 times greater, 35 to 80 times greater, 20 to 75 times greater, 25 to 70 times greater, 40 to 100 times greater, 50 to 100 times greater, 55 to 95 times greater, 45 to 90 times greater, 40 to 70 times greater, 18 to 50 times greater, 18 to 40 times greater, 18 to 35 times greater, or 18 to 30 times greater, by weight, pentazocine hydrochloride salt. Non-limiting examples of the methods of the present invention comprise of intravenous administration of 10 mg of pentazocine hydrochloride salt with 0.4 mg of naloxone hydrochloride salt (i.e., 25 times greater, by weight, pentazocine hydrochloride salt than naloxone hydrochloride salt), 15 mg of pentazocine hydrochloride salt with 0.3 mg naloxone hydrochloride salt (i.e., 30 times greater, by weight, pentazocine hydrochloride salt than naloxone hydrochloride salt), and 25 mg of pentazocine hydrochloride salt with 0.5 mg naloxone hydrochloride salt (i.e., 50 times greater pentazocine hydrochloride salt than naloxone hydrochloride salt).

In specific embodiments of the invention, the weight of pentazocine hydrochloride salt administered intravenously is 3 mg to 50 mg, 4 mg to 50 mg, 5 mg to 50 mg, 6 mg to 50 mg, 7 mg to 50 mg, 3 mg to 45 mg, 5 mg to 45 mg, 10 mg to 45 mg, 15 mg to 45 mg, 5 mg to 40 mg, 10 mg to 40 mg, 3 mg to 35 mg, 4 mg to 35 mg, 5 mg to 35 mg, 10 mg to 35 mg, 3 mg to 30 mg, 4 mg to 30 mg, 5 mg to 30 mg, 3 mg to 25 mg, 4 mg to 25 mg, 3 mg to 20 mg, 4 mg to 20 mg, 5 mg to 25 mg, 10 mg to 25 mg, 15 mg to 30 mg, 15 mg to 25 mg, 10 mg to 20 mg, and 10 mg to 15 mg.

In certain embodiments in which pentazocine hydrochloride salt is administered intravenously, naloxone hydrochloride is preferably administered as the opioid antagonist hydrochloride salt in amounts of 0.02 mg to 8 mg, 0.02 to 7 mg, 0.02 mg to 6 mg, 0.02 to 5 mg, 0.02 to 4 mg, 0.02 to 3 mg, 0.02 to 2 mg, 0.02 to 2 mg, 0.1 mg to 8 mg, 0.1 mg to 7 mg, 0.1 mg to 6 mg, 0.1 mg to 5 mg, 0.1 mg to 4 mg, 0.1 mg to 3 mg, 0.1 mg to 2 mg, 0.1 mg to 1 mg, 0.2 mg to 8 mg, 0.2 mg to 7 mg, 0.2 mg to 6 mg, 0.2 mg to 4 mg, 0.2 mg to 3 mg, 0.2 mg to 2 mg, 0.2 mg to 1 mg, 0.4 mg to 8 mg, 0.4 to 7 mg, 0.4 to 6 mg, 0.4 mg to 5 mg, 0.4 mg to 4 mg, 0.4 mg to 3 mg, 0.4 mg to 2 mg, 0.4 mg to 1 mg, 0.5 mg to 8 mg, 0.5 mg to 6 mg, 0.5 mg to 5 mg, 0.5 mg to 4 mg, 0.5 mg to 3 mg, 0.5 mg to 3 mg, 0.5 to 2 mg, 0.5 mg to 1 mg, 1 mg to 8 mg, 1 mg to 6 mg, 1 mg to 5 mg, 1 mg to 4 mg, 1 mg to 3 mg, 1 mg to 2 mg, 5 mg to 8 mg, 4 mg to 7 mg, 3 mg to 5 mg, 0.02 mg to 1 mg, preferably 0.1 mg to 0.8 mg, 0.2 mg to 0.8 mg, 0.3 mg to 0.8 mg, 0.4 mg to 0.8 mg, 0.5 mg to 0.8 mg, 0.1 mg to 0.7 mg, 0.2 mg to 0.7 mg, 0.3 mg to 0.7 mg, 0.4 mg to 0.7 mg, 0.1 mg to 0.6 mg, 0.2 mg to 0.6 mg, 0.3 to 0.6 mg, 0.1 mg to 0.5 mg, 0.15 mg to 0.5 mg, 0.2 mg to 0.5 mg, 0.25 mg to 0.5 mg, 0.3 mg to 0.5 mg, 0.35 mg to 0.5 mg, 0.1 mg to 0.45 mg, 0.15 mg to 0.45 mg, 0.2 mg to 0.45 mg, 0.25 mg to 0.45 mg, 0.1 mg to 0.3 mg, 0.13 mg to 0.3 mg, 0.2 mg to 0.3 mg, 0.1 mg to 0.25 mg, 0.15 mg to 0.25 mg, 0.1 mg to 0.2 mg, 0.1 mg to 0.15 mg. Non-limiting examples of the present invention are methods comprising administration of 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 30 mg, 35 mg, 40 mg, 45 mg, and 50 mg pentazocine hydrochloride salt with 0.1 mg, 0.15 mg, 0.2 mg, 0.25 mg, 0.3 mg, 0.35 mg, 0.4 mg, 0.45 mg, 0.5 mg, 0.55 mg, 0.6 mg 0.7 mg, 0.75 mg, 0.8 mg, 1 mg, 1.6 mg, 2 mg, 2.4 mg, 3 mg, 4 mg, 5 mg, 6 mg, 7 mg and 8 mg naloxone hydrochloride salt.

In another particular embodiment of the invention, butorphanol free base, a pharmaceutically acceptable salt, a prodrug, or a mixture thereof is administered with an opioid antagonist to achieve greater analgesia than that produced by administration of the butorphanol or the antagonist alone. The butorphanol tartrate salt form is preferably administered at 0.3 to 10 times greater, 0.3 to 9 times greater, 0.3 to 8 times greater, 0.5 to 10 times greater, 0.3 to 7 times greater, 0.5 to 7 times greater, 0.3 to 6 times greater. 0.5 to 6 times greater, 0.3 to 5 times greater, 0.5 to 5 times greater, 0.3 to 4 times greater, 0.5 to 4 times greater, 0.3 to 3 times greater, or 0.5 to 3 times greater, by weight, than the amount of opioid antagonist hydrochloride salt administered. Non-limiting examples of the present invention are methods comprising administration of 0.3 mg of butorphanol tartrate salt with 0.3 mg of naloxone hydrochloride salt (i.e., 1 time greater, by weight, of butorphanol tartrate salt than naloxone hydrochloride salt), 0.5 mg of butorphanol tartrate salt with 0.2 mg naloxone hydrochloride salt (i.e., 2.5 times greater, by weight, butorphanol tartrate salt than naloxone hydrochloride salt), and 0.8 mg of butorphanol tartrate salt with 0.4 mg naloxone hydrochloride salt (i.e., 2 times greater, by weight butorphanol tartrate salt than naloxone hydrochloride salt).

In specific embodiments, the method of the invention comprises intravenous administration of butorphanol tartrate salt as the κ-opioid agonist. In more specific embodiments, the weight of butorphanol tartrate salt administered intravenously is 0.2 mg to 2 mg, 0.2 mg to 1.9 mg, 0.2 mg to 1.8 mg, 0.2 mg to 1.7 mg, 0.25 mg to 1.9 mg, 0.25 mg to 1.8 mg, 0.25 mg to 1.75 mg, 0.25 mg to 1.5 mg, 0.25 mg to 1 mg, or 0.2 mg to 1 mg. In certain preferred embodiments comprising intravenous administration of butorphanol tartrate salt, 0.02 mg to 8 mg, 0.02 to 7 mg, 0.02 mg to 6 mg, 0.02 to 5 mg, 0.02 to 4 mg, 0.02 to 3 mg, 0.02 to 2 mg, 0.02 to 2 mg, 0.1 mg to 8 mg, 0.1 mg to 7 mg, 0.1 mg to 6 mg, 0.1 mg to 5 mg, 0.1 mg to 4 mg, 0.1 mg to 3 mg, 0.1 mg to 2 mg, 0.1 mg to 1 mg, 0.2 mg to 8 mg, 0.2 mg to 7 mg, 0.2 mg to 6 mg, 0.2 mg to 4 mg, 0.2 mg to 3 mg, 0.2 mg to 2 mg, 0.2 mg to 1 mg, 0.4 mg to 8 mg, 0.4 to 7 mg, 0.4 to 6 mg, 0.4 mg to 5 mg, 0.4 mg to 4 mg, 0.4 mg to 3 mg, 0.4 mg to 2 mg, 0.4 mg to 1 mg, 0.5 mg to 8 mg, 0.5 mg to 6 mg, 0.5 mg to 5 mg, 0.5 mg to 4 mg, 0.5 mg to 3 mg, 0.5 mg to 3 mg, 0.5 to 2 mg, 0.5 mg to 1 mg, 1 mg to 8 mg, 1 mg to 6 mg, 1 mg to 5 mg, 1 mg to 4 mg, 1 mg to 3 mg, 1 mg to 2 mg, 5 mg to 8 mg, 4 mg to 7 mg, 3 mg to 5 mg, 0.02 mg to 1 mg, preferably 0.1 mg to 0.8 mg, 0.2 mg to 0.8 mg, 0.3 mg to 0.8 mg, 0.4 mg to 0.8 mg, 0.5 mg to 0.8 mg, 0.1 mg to 0.7 mg, 0.2 mg to 0.7 mg, 0.3 mg to 0.7 mg, 0.4 mg to 0.7 mg, 0.1 mg to 0.6 mg, 0.2 mg to 0.6 mg, 0.3 to 0.6 mg, 0.1 mg to 0.5 mg, 0.15 mg to 0.5 mg, 0.2 mg to 0.5 mg, 0.25 mg to 0.5 mg, 0.3 mg to 0.5 mg, 0.35 mg to 0.5 mg, 0.1 mg to 0.45 mg, 0.15 mg to 0.45 mg, 0.2 mg to 0.45 mg, 0.25 mg to 0.45 mg, 0.1 mg to 0.3 mg, 0.13 mg to 0.3 mg, 0.2 mg to 0.3 mg, 0.1 mg to 0.25 mg, 0.15 mg to 0.25 mg, 0.1 mg to 0.2 mg, 0.1 mg to 0.15 mg naloxone hydrochloride salt is administered as the opioid antagonist hydrochloride. Non-limiting examples of the present invention are methods comprising intravenous administration of 0.2 mg, 0.3 mg, 0.4 mg, 0.5 mg, 0.6 mg, 0.7 mg, 0.8 mg, 0.9 mg, 1.0 mg, 1.1 mg, 1.1 mg, 1.2 mg, 1.3 mg, 1.4 mg, 1.5 mg, 1.6 mg, 1.7 mg, 1.8 mg, or 2.0 mg of butorphanol tartrate salt with 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.7 mg, 0.75 mg, 0.8 mg, 1 mg, 1.6 mg, 2 mg, 2.4 mg, 3 mg, 4 mg, 5 mg, 6 mg, 7 mg and 8 mg naloxone hydrochloride salt. The preferred embodiments of the present invention also encompass methods comprising administration of butorphanol free base, non-tartrate salt, prodrug, or mixture thereof in an equivalent amount to butorphanol tartrate salt and/or administration of the opioid antagonist free base, non-hydrochloride salt, prodrug, or mixture thereof in an equivalent amount to the opioid antagonist hydrochloride salt.

The present invention also encompasses methods of treating pain comprising administration by a method other than intravenous (and, in certain embodiments, also other than oral administration for gastro-intestinal uptake) an amount of a κ-opioid receptor antagonist and an amount of an opioid agonist that results in greater analgesia than the administration of the opioid agonist or antagonist alone. The formulation of the specific embodiment of the invention should suit the particular mode of administration.

In certain embodiments of the present invention, the method of treating, managing, and ameliorating pain comprises administration by a method, other than intravenous administration (and in certain embodiments, also other than oral administration for gastro-intestinal uptake) an opioid antagonist free base, salt, prodrug, or mixture thereof and an nalbuphine free base, salt, prodrug, or mixture thereof in equivalent amounts to intravenous administration of the opioid antagonist hydrochloride salt and 6.25 to 49 times greater, 6.25 to 40 times greater, 6.25 to 35 times greater, 6.25 to 30 times greater, 6.25 to 20 times greater, 8 to 35 times greater, 8 to 30 times greater, 8 to 25 times greater, 10 to 30 times greater, 10 to 20 times greater, 15 to 25 times greater, 20 to 25 times greater, 10 to 15 times greater, 9 to 15 times greater, 5 to 10 times greater, 30 to 49 times greater, 20 to 30 times greater, 15 to 20 times greater, 13 to 15 times greater, 11 to 13 times greater, 9 to 11 times greater, 7 to 9 times greater, or 6.25 to 7 times greater, by weight, nalbuphine hydrochloride salt.

Alternatively, nalbuphine free base, salt, prodrug, or mixture thereof is administered by a method other than intravenously in amounts equivalent to 0.02 mg to 8 mg, 0.02 to 7 mg, 0.02 mg to 6 mg, 0.02 to 5 mg, 0.02 to 4 mg, 0.02 to 3 mg, 0.02 to 2 mg, 0.02 to 2 mg, 0.1 mg to 8 mg, 0.1 mg to 7 mg, 0.1 mg to 6 mg, 0.1 mg to 5 mg, 0.1 mg to 4 mg, 0.1 mg to 3 mg, 0.1 mg to 2 mg, 0.1 mg to 1 mg, 0.2 mg to 8 mg, 0.2 mg to 7 mg, 0.2 mg to 6 mg, 0.2 mg to 4 mg, 0.2 mg to 3 mg, 0.2 mg to 2 mg, 0.2 mg to 1 mg, 0.4 mg to 8 mg, 0.4 to 7 mg, 0.4 to 6 mg, 0.4 mg to 5 mg, 0.4 mg to 4 mg, 0.4 mg to 3 mg, 0.4 mg to 2 mg, 0.4 mg to 1 mg, 0.5 mg to 8 mg, 0.5 mg to 6 mg, 0.5 mg to 5 mg, 0.5 mg to 4 mg, 0.5 mg to 3 mg, 0.5 mg to 3 mg, 0.5 to 2 mg, 0.5 mg to 1 mg, 1 mg to 8 mg, 1 mg to 6 mg, 1 mg to 5 mg, 1 mg to 4 mg, 1 mg to 3 mg, 1 mg to 2 mg, 5 mg to 8 mg, 4 mg to 7 mg, 3 mg to 5 mg, 0.02 mg to 1 mg, preferably 0.1 mg to 0.8 mg, 0.2 mg to 0.8 mg, 0.3 mg to 0.8 mg, 0.4 mg to 0.8 mg, 0.5 mg to 0.8 mg, 0.1 mg to 0.7 mg, 0.2 mg to 0.7 mg, 0.3 mg to 0.7 mg, 0.4 mg to 0.7 mg, 0.1 mg to 0.6 mg, 0.2 mg to 0.6 mg, 0.3 to 0.6 mg, 0.1 mg to 0.5 mg, 0.15 mg to 0.5 mg, 0.2 mg to 0.5 mg, 0.25 mg to 0.5 mg, 0.3 mg to 0.5 mg, 0.35 mg to 0.5 mg, 0.1 mg to 0.45 mg, 0.15 mg to 0.45 mg, 0.2 mg to 0.45 mg, 0.25 mg to 0.45 mg, 0.1 mg to 0.3 mg, 0.13 mg to 0.3 mg, 0.2 mg to 0.3 mg, 0.1 mg to 0.25 mg, 0.15 mg to 0.25 mg, 0.1 mg to 0.2 mg, 0.1 mg to 0.15 mg nalbuphine hydrochloride salt or equivalent amount of free base, non-hydrochloride salt, prodrug, or mixture thereof intravenously administered.

An amount of a κ-opioid agonist and an opioid antagonist administered by a method other than intravenous administration and an amount of a κ-opioid opioid agonist and an opioid antagonist administered intravenously are equivalent amounts if both amounts produce approximately the same blood concentration of κ-opioid agonist and opioid antagonist after administration. Any method in the art may be used to measure the blood concentration. For example, but not by way of limitation, blood concentration of κ-opioid agonists and opioid antagonists may be measured by high-performance liquid chromatography. Blood concentration of κ-opioid agonists and antagonists may be measured immediately after administration, less than or at approximately 10 minutes after administration, less than or at approximately 15 minutes after administration, less than or at approximately 20 minutes after administration, less than or at approximately 30 minutes after administration, less than or at approximately 45 minutes after administration, less than or at approximately 1 hour after administration, less than or at approximately 2 hours after administration, less than or approximately 3 hours after administration, less than or approximately 4 hours after administration, less than or approximately 5 hours after administration, or less than or approximately 6 hours after administration.

Depending on the specific non-intravenous mode of administration, a dose for non-intravenous administration may comprise a ratio of κ-opioid agonist to opioid antagonist that is lesser, equal, or greater than the ratio of κ-opioid agonist to opioid agonist in an equivalent dose for intravenous administration. Also depending on the specific non-intravenous mode of administration, a dose for non-intravenous administration may comprise great amounts of κ-opioid agonist and opioid antagonist, by weight, than an equivalent dose for intravenous administration.

In certain embodiments of the invention, the method comprises sublingual administration (or other oral cavity administration), the opioid antagonist hydrochloride salt is administered with 1 to 60 times greater, 1 to 50 times greater, 1 to 45 times greater, 1 to 40 times greater, 5 to 50 times greater, 5 to 40 times greater, 5 to 35 times greater, 10 to 40 times greater, 15 to 40 times greater, 10 to 30 times greater, 15 to 30 times greater, 1 to 30 times greater, 1 to 20 times greater, 1 to 15 times greater, or 1 to 9 times greater nalbuphine hydrochloride salt is administered sublingually. Non-limiting examples of methods of the present invention comprise sublingual administration of 8 mg nalbuphine hydrochloride salt with 0.4 mg naloxone hydrochloride salt (i.e., 20 times greater, by weight, nalbuphine hydrochloride salt than naloxone hydrochloride salt), 15 mg nalbuphine hydrochloride salt with 3 mg naloxone hydrochloride salt (i.e., 5 times greater, by weight, nalbuphine hydrochloride salt than naloxone hydrochloride salt), and 30 mg nalbuphine hydrochloride salt with 4 mg naloxone hydrochloride salt (i.e., 7.5 times greater, by weight, nalbuphine hydrochloride salt than naloxone hydrochloride salt). Alternatively, 5 mg to 65 mg, 5 mg to 60 mg, 5 mg to 55 mg, 5 mg to 50 mg, 5 mg to 40 mg, 5 mg to 35 mg, 6 mg to 55 mg, 6 mg to 50 mg, 6 mg to 45 mg, 6 mg to 40 mg, 6 mg to 30 mg, 7 mg to 40 mg, 7 mg to 35 mg, or 7.5 to 30 mg of nalbuphine hydrochloride salt is administered sublingually.

In certain preferred embodiments of the invention in which the method comprises sublingual administration of nalbuphine hydrochloride salt, naloxone hydrochloride salt is preferably administered sublingually as the opioid antagonist hydrochloride in amounts preferably 0.1 mg to 10 mg, 0.1 mg to 9 mg, 0.1 mg to 8 mg, 0.1 mg to 7 mg, 0.1 mg to 6 mg, 0.2 mg to 10 mg, 0.2 mg to 9 mg, 0.2 mg to 8 mg, 0.2 mg to 7 mg, 0.2 mg to 6 mg, 0.2 mg to 5 mg, 0.3 mg to 10 mg, 0.3 mg to 9 mg, 0.3 mg to 7 mg, 0.3 mg to 6 mg, 0.3 mg to 5 mg, 0.3 mg to 4 mg, 0.4 mg to 4 mg, 0.5 mg to 4 mg, 0.4 mg to 3.5 mg, 0.5 mg to 3.5 mg, 0.6 mg to 3.5 mg, 0.4 mg to 3 mg, 0.5 mg to 3 mg, 0.4 mg to 2.8 mg, 0.6 mg to 2.7 mg, 0.4 mg to 2.5 mg, 0.5 mg to 2.2 mg, 0.4 mg to 2 mg, 0.6 mg to 2 mg, 0.4 mg to 1.5 mg, or 0.4 mg to 1 mg. Non-limiting examples of the present invention are methods comprising administration of 5 mg, 5.5 mg, 6 mg, 6.5 mg, 7 mg, 7.5 mg, 8 mg, 8.5 mg, 9 mg, 9.5 mg 10 mg, 15 mg, 20 mg, 30 mg, 35 mg, 40 mg, 45 mg or 50 mg nalbuphine hydrochloride salt with naloxone hydrochloride salt.

For example, in specific embodiments of the present invention, the method comprises sublingual administration of 0.4 mg of naloxone hydrochloride salt with 5 mg of nalbuphine hydrochloride salt, 0.4 mg of naloxone hydrochloride salt with 6 mg of nalbuphine hydrochloride salt, 0.4 mg of naloxone hydrochloride salt with 7 mg of nalbuphine hydrochloride salt, 0.4 mg of naloxone hydrochloride salt with 8 mg of nalbuphine hydrochloride salt, 0.4 mg of naloxone hydrochloride salt with 9 mg of nalbuphine hydrochloride salt, 0.4 mg of naloxone hydrochloride salt with 10 mg of nalbuphine hydrochloride salt, 0.4 mg of naloxone hydrochloride salt with 15 mg of nalbuphine hydrochloride salt, 0.4 mg of naloxone hydrochloride salt with 20 mg of nalbuphine hydrochloride salt, 0.4 mg of naloxone hydrochloride salt with 25 mg of nalbuphine hydrochloride salt, and 0.4 mg of naloxone hydrochloride salt with 30 mg of nalbuphine hydrochloride salt.

The present invention further encompasses methods comprising administration of equivalent amounts of nalbuphine free base, non-hydrochloride salt, prodrug or mixture thereof to nalbuphine hydrochloride salt and equivalent amounts of the opioid antagonist free base, non-hydrochloride salt, prodrug, or mixture thereof to the opioid hydrochloride salt.

The present invention also encompasses methods comprising administration, other than intravenous administration (and, in certain embodiments, other than oral administration for gastro-intestinal uptake), pentazocine, a prodrug, a pharmaceutically acceptable salt, or mixture thereof with an opioid antagonist, prodrug, a pharmaceutically acceptable salt, or mixture thereof in amounts equivalent to intravenous administration of an opioid antagonist hydrochloride salt, preferably naloxone hydrochloride salt, and 18 to 120 times greater, 25 to 120 times greater, 18 to 110 times greater, 25 to 110 times greater. 18 to 100 times greater, 25 to 100 times greater, 18 to 95 times greater, 25 to 90 times greater, 30 to 90 times greater, 35 to 90 times greater, 18 to 80 times greater, 20 to 80 times greater, 20 to 60 times greater, 20 to 50 times greater, 25 to 55 times greater, 35 to 80 times greater, 20 to 75 times greater, 25 to 70 times greater, 40 to 100 times greater, 50 to 100 times greater, 55 to 95 times greater, 45 to 90 times greater, 40 to 70 times greater, 18 to 50 times greater, 18 to 40 times greater, 18 to 35 times greater, or 18 to 30 times greater, by weight, pentazocine hydrochloride salt than the opioid antagonist hydrochloride salt, preferably naloxone hydrochloride salt. Alternatively, the amount of pentazocine, a prodrug, a pharmaceutically acceptable salt, or mixture thereof administered is an amount equivalent to intravenous administration of 3 mg to 50 mg, 4 mg to 50 mg, 5 mg to 50 mg, 6 mg to 50 mg, 7 mg to 50 mg, 3 mg to 45 mg, 5 mg to 45 mg, 10 mg to 45 mg, 15 mg to 45 mg, 5 mg to 40 mg, 10 mg to 40 mg, 3 mg to 35 mg, 4 mg to 35 mg, 5 mg to 35 mg, 10 mg to 35 mg, 3 mg to 30 mg, 4 mg to 30 mg, 5 mg to 30 mg, 3 mg to 25 mg, 4 mg to 25 mg, 3 mg to 20 mg, 4 mg to 20 mg, 5 mg to 25 mg, 10 mg to 25 mg, 15 mg to 30 mg, 15 mg to 25 mg, 10 mg to 20 mg, and 10 mg to 15 mg pentazocine hydrochloride salt.

In certain embodiments comprising sublingual administration (or other oral cavity administration) of pentazocine, the amount of pentazocine hydrochloride salt 7.5 to 50 times greater, 7.5 to 45 times greater, 10 to 50 times greater, 10 to 40 times greater, 15 to 50 times greater, 15 to 45 times greater, 7.5 to 30 times greater, 7.5 to 25 times greater, 7.5 to 20 times greater, 10 to 30 times greater, or 7.5 to 15 times greater, by weight, than the amount of opioid antagonist hydrochloride salt administered.

Alternatively, the amount of pentazocine hydrochloride salt administered sublingually (or by other oral cavity administration) is 30 mg to 100 mg, 30 to 90 mg, 40 mg to 100 mg, 30 mg to 85 mg, 40 mg to 85 mg, 30 mg to 70 mg, 40 mg to 70 mg, 30 mg to 60 mg, 40 mg to 60 mg, 30 mg to 50 mg, 50 mg to 80, or 30 mg to 45 mg. In embodiments of the invention in which pentazocine hydrochloride salt is administered sublingually (or by other oral cavity administration), naloxone hydrochloride salt is the preferred opioid antagonist hydrochloride salt and is administered in amounts 1 mg to 10 mg, 1 mg to 9 mg, 1 mg to 8 mg, 1 mg to 7 mg, 1 mg to 6 mg, 1 mg to 5 mg, 1 mg to 4 mg, 2 mg to 10 mg, 2 mg to 9 mg; 2 mg to 8 mg, 2 mg to 7 mg, 2 mg to 6 mg, 2 mg to 5 mg, 2 mg to 4 mg, 2 mg to 3.8 mg, 2 mg to 3.6 mg, 2 mg to 3.4 mg, 2 mg to 3.4 mg, 2.2 mg to 3.5 mg, 2 mg to 3.2 mg, 3 mg to 4 mg, or 2 mg to 3 mg. Non-limiting examples of the present invention are methods comprising sublingual administration of 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 50 mg, 55 mg, 60 mg, 65 mg, 70 mg, 75 mg, 80 mg, 85 mg, 90 mg, 95 mg or 100 mg pentazocine hydrochloride salt with 2 mg, 2.1 mg, 2.2 mg, 2.3 mg, 2.4 mg, 2.5 mg, 2.6 mg, 2.7 mg, 2.8 mg, 2.9 g, 3 mg, 3.1 mg, 3.2 mg, 3.3 mg, 3.4 mg, 3.5 mg, 3.6 mg, 3.7 mg, 3.8 mg, 3.9 mg, or 4.0 mg naloxone hydrochloride salt.

The present invention further encompasses methods comprising administration of equivalent amounts of pentazocine free base, non-hydrochloride salt, prodrug or mixture thereof to pentazocine hydrochloride salt and equivalent amounts of the opioid antagonist free base, non-hydrochloride salt, prodrug, or mixture thereof to the opioid hydrochloride salt, provided that if the method of administration is oral for gastro-intestinal uptake, 50 mg pentazocine hydrochloride is not administered with 0.5 mg naloxone hydrochloride.

In certain preferred embodiments, butorphanol, a prodrug, a pharmaceutically acceptable salt, or mixture thereof is administered, by a method other than intravenous administration (and, in certain embodiments, other than oral administration for gastro-intestinal uptake), with an opioid antagonist, pharmaceutically acceptable salt, prodrug, or mixture thereof in amounts equivalent to intravenous administration the opioid antagonist hydrochloride salt, preferably naloxone hydrochloride salt, and 0.3 to 10 times greater, 0.3 to 9 times greater, 0.3 to 8 times greater, 0.5 to 10 times greater, 0.3 to 7 times greater, 0.5 to 7 times greater, 0.3 to 6 times greater. 0.5 to 6 times greater, 0.3 to 5 times greater, 0.5 to 5 times greater, 0.3 to 4 times greater, 0.5 to 4 times greater, 0.3 to 3 times greater, or 0.5 to 3 times greater, by weight, of butorphanol tartrate salt than the opioid antagonist hydrochloride salt.

Alternatively, the amount of butorphanol, a prodrug, a pharmaceutically acceptable salt, or mixture thereof is administered by a method other than intravenous administration (and, in certain embodiments, other than oral administration for gastro-intestinal uptake) in amounts equivalent to 0.2 mg to 2 mg, 0.2 mg to 1.9 mg, 0.2 mg to 1.8 mg, 0.2 mg to 1.7 mg, 0.25 mg to 1.9 mg, 0.25 mg to 1.8 mg, 0.25 mg to 1.75 mg, 0.25 mg to 1.5 mg, 0.25 mg to 1 mg, or 0.2 mg to 1 mg of butorphanol tartrate salt administered intravenously. In certain embodiments comprising sublingual administration (or other oral cavity administration) of butorphanol as the κ-opioid agonist, butorphanol tartrate salt is administered in amounts 0.1 to 60 times greater, 0.1 to 50 times greater, 0.1 to 45 times greater, 0.3 to 40 times greater, or 0.5 to 30 greater, 10 to 60 times greater, 20 to 50 times greater, or 10 to 30 times greater by weight, than the opioid antagonist hydrochloride salt, preferably naloxone hydrochloride salt, administered. Non-limiting examples of the present invention are methods comprising sublingual administration of 0.5 mg butorphanol tartrate salt with 0.25 mg naloxone hydrochloride salt (i.e., 2 times greater, by weight, butorphanol tartrate salt than naloxone hydrochloride salt), 2 mg butorphanol tartrate salt with 0.2 mg naloxone hydrochloride salt (i.e., 10 times greater, by weight, butorphanol tartrate salt than naloxone hydrochloride salt), and 6 mg butorphanol tartrate salt with 0.3 mg naloxone hydrochloride salt (i.e., 20 times greater, by weight, butorphanol tartrate salt than naloxone hydrochloride salt). Alternatively, 0.1 mg to 10 mg, 0.1 mg to 9 mg, 0.1 mg to 8 mg, 0.1 mg to 7 mg, 0.1 mg to 6 mg, 0.2 mg to 9 mg, 0.2 mg to 8 mg, 0.2 mg to 7 mg, 0.2 mg to 6 mg, 0.2 mg to 5.5 mg, 0.3 to 6.5 mg, 0.4 mg to 7 mg, or 0.5 mg to 6 mg of butorphanol tartrate salt is administered sublingually.

If the κ-opioid agonist is butorphanol tartrate salt, then, preferably, 0.1 to 4 mg, 0.1 mg to 3.5 mg, 0.1 mg to 3 mg, 0.1 to 2.5 mg, 0.1 mg to 2 mg, 0.1 mg to 1 mg, 0.3 mg to 0.8 mg, or 0.1 mg to 0.8 mg of naloxone hydrochloride salt is administered. Non-limiting examples of the present invention are methods comprising administration of 0.1 mg, 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, or 7 mg butorphanol tartrate salt with 0.1 mg, 0.2 mg, 0.3 mg, 0.4 mg, 0.5 mg, 0.6 mg, 0.7 mg, or 0.85 mg naloxone hydrochloride salt.

The present invention further encompasses pharmaceutical compositions comprising equivalent amounts of butorphanol free base, non-tartrate salt, prodrug or mixture thereof to butorphanol tartrate salt and equivalent amounts of the opioid antagonist free base, non-hydrochloride salt, prodrug, or mixture thereof to the opioid hydrochloride salt.

### Combinational Therapies

In certain embodiments of the present invention, the composition of the invention can be used in combination therapy with at least one other therapeutic agent. The compound of the invention and the therapeutic agent can act additively or, more preferably, synergistically. In a preferred embodiment, a composition of the invention is administered concurrently with the administration of another therapeutic agent, which can be part of the same composition as or in a different composition from that comprising the combination of 6 opioid receptor agonist and opioid antagonist of the invention. In another embodiment, a combination of the invention is administered prior or subsequent to administration of another therapeutic agent. In one embodiment of combination therapy that involves treatment of chronic pain, the combination therapy involves alternating between administering a composition comprising a composition of the invention and a composition comprising another therapeutic agent, e.g., to minimize the toxicity associated with a particular drug. The duration of administration of the composition of the invention or therapeutic agent can be, e.g., one month, three months, six months, a year, or for more extended periods. In certain embodiments, when a compound of the invention is administered concurrently with another therapeutic agent that potentially produces adverse side effects including, but not limited to, toxicity, the therapeutic agent can advantageously be administered at a dose that falls below the threshold at which the adverse side is elicited.

In certain embodiments, the compositions of the present invention can be combined in dosage forms with non-opioid analgesics, e.g., non-steroidal anti-inflammatory agents, including aspirin, ibuprofen, diclofenac, naproxen, benoxaporfen, flurbiprofen, fenoprofen, flubufen, ketoprofen, indoprofen, piroprofen, carporfen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiaza, clinndanac, oxpinac, mefenamic acid, meclofenamic acid, flufenamic acid, niflumic acid, tolfenamic acid, diflurisal, fluefenisal, piroxicam, sudoxicam, or isocxicam, or pharmaceutically acceptable salts, prodrugs, or mixtures thereof. Other suitable non-opioid analgesic which may be included in dosage forms of the present invention include the following, non-limiting chemical classes of analgesics, antipyretic, nonsteroidal anti-inflammatory drugs (NSAIDs) (e.g., aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), sulindac (CLINORIL™), tolmentin (TOLECTIN™), rofecoxib (VIOXX™), naproxen (ALEVE™, NAPROSYN™), ketoprofen (ACTRON™), and nabumetone (RELAFEN™)); steroidal anti-inflammatory drugs include, but are not limited to, glucocorticoids, dexamethasone (DECADRON™), cortisone, hydrocortisone, prednisone (DELTASON™), prednisolone, triamcinolone, azulfindine, and eicosanoids, such as prostaglandins, thromboxanes, and leukortrienes; salicylic acid derivatives, including aspirin, sodium salicylate, choline magnesium trisalicylate, salsalate, diflunisal, salicylsalicylic acid, sulfasalazine, and olsalazin; para-aminophenol derivatives including acetaminophen and phenacetin; indole and indene acetic acids, including indomethacin, sulindac, and etodolac; heteroaryl acetic acids, including tolmetin, diclorfenac, and ketorolac; anthranilic acids (e.g., fenamates), including mefanamic acid, and meclofenamic acid; enolic acids, including oxicams (e.g., prioxicam or tenoxicam), and pyrazolidinediones (e.g., phenylbutazone or oxyphenthartazone); and alkanones, including nabumetone.

In certain embodiments, the compounds of the present invention can be formulated in a pharmaceutical dosage form in combination with antimigraine agents. Antimigraine agents include, but are not limited to, alpiropride, dihydroergotamine, dolasetron, ergocornine, ergocorninine, ergocryptine, ergot, ergotamine, flumedroxone acetate, fonazine, lisuride, lomerizine, methysergide oxetorone, pizotyline, and mixtures thereof.

In certain embodiments, the compounds of the present invention can be formulated in a pharmaceutical dosage form in combination with antidepressants. Suitable antidepressants include, but are not limited to, binedaline, caroxazone, citalopram, dimethazan, fencamine, indalpine, indeloxazine hydrochloride, nefopam, nomifensine, oxitriptan, oxypertine, paroxetine, sertraline, thiazesim, trazodone, benmoxine, iproclozide, iproniazid, isocarboxazid, nialamide, octamoxin, phenelzine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, and zimeldine.

In certain embodiments of the present invention, the composition of the invention can be combined with antiepileptic drugs, e.g., phenyloin (DILANTIN™), which is usually the drug of choice for the treatment of epilepsy, phenobarbital, primidone (MYSOLINE™), carbamazepine (TEGRETOL™) for complex partial tonic-clonic seizures, and ethosuximide (ZARONTIN™) and clonazepam (KLONOPIN™) for absence seizures.

### Pharmaceutical Kits

The present invention also encompasses pharmaceutical kits for the practice of the methods of this invention. The kits include one or more κ-opioid receptor agonists and one or more opioid receptor antagonists in dosages and amounts used in the methods and compositions of the invention. The κ-opioid receptor agonists and the opioid antagonists may be provided in the free base, pharmaceutically acceptable salt, prodrug, or mixture thereof in dosages and amounts used in the methods and compositions of the invention. The kit may also comprise of additional active and inactive ingredients. The kit may further comprise of devises that are used to administer the compounds of the invention. Examples of such devices include, but are not limited to, syringes, drip bags, patches, and inhalers. In some embodiments the kits may comprise nasal spray bottles or inhalers, or the like, which may either be charged with compositions to be dispensed or which may be accompanied by said compositions in a separate package or container.

In certain embodiments, the kits additionally include instructional materials teaching the methods of treating, managing, or ameliorating pain of the present invention. The instructional material may comprise of written or printed materials or in any medium capable of storing such instructions and communicating them to the end user. Such media include, but are not limited to electronic storage media (e.g., magnetic discs, tapes, cartridges, chips), optical media (e.g., CD ROM), and the like. Such media may include addresses to internet sites that provide such instructional materials.

The following examples are set forth to assist in understanding the invention and should not be construed as specifically limiting the invention described and claimed herein. Such variations, including the substitutions of all equivalents now known or later developed, which would be within the purview of those skilled in the art, and changes in formulations or minor changes in methods or experimental designs, are to be considered to fall within the scope of the invention incorporated herein.

### EXAMPLES

### Example 1

In this clinical trial, patients underwent standardized surgery by the same oral surgeon for removal of third molar (“wisdom”) teeth, including at least one bony impacted mandibular third molar. Prior to surgery patients received intravenous diazepam, nitrous oxide, and a local anesthetic (mepivacaine without vasoconstrictor to obtain a nerve block of short duration). After surgery, each patient was randomly assigned to receive, in an open injection, double-blinded fashion, through an intravenous line, an injection of either naloxone hydrochloride salt or a mixture of naloxone hydrochloride salt and nalbuphine hydrochloride salt (Abbott Laboratories, Abbott Park, Ill.).

Criteria for administration of the test drug were an elapse of a period of at least 80 minutes after the onset of the local anesthetic and a pain rating that was greater than one quarter (2.5 cm) of the maximum possible visual analog scale (VAS) rating (10 cm). Baseline pain intensity was defined as the last VAS pain rating before administration of the test drug or drugs. VAS pain ratings were recorded at 20 minute intervals beginning ten minutes after administration of the test drug, was three hours. For each patient, the magnitude of the analgesic response was defined as the difference between the pain rating at each time point following test drug administration and the baseline VAS pain rating.

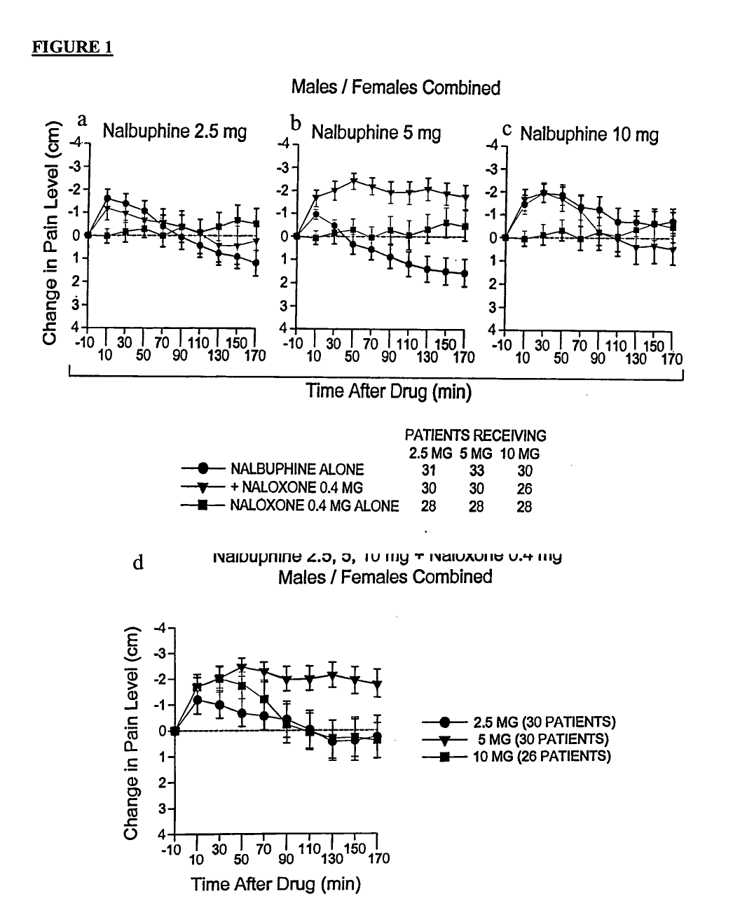

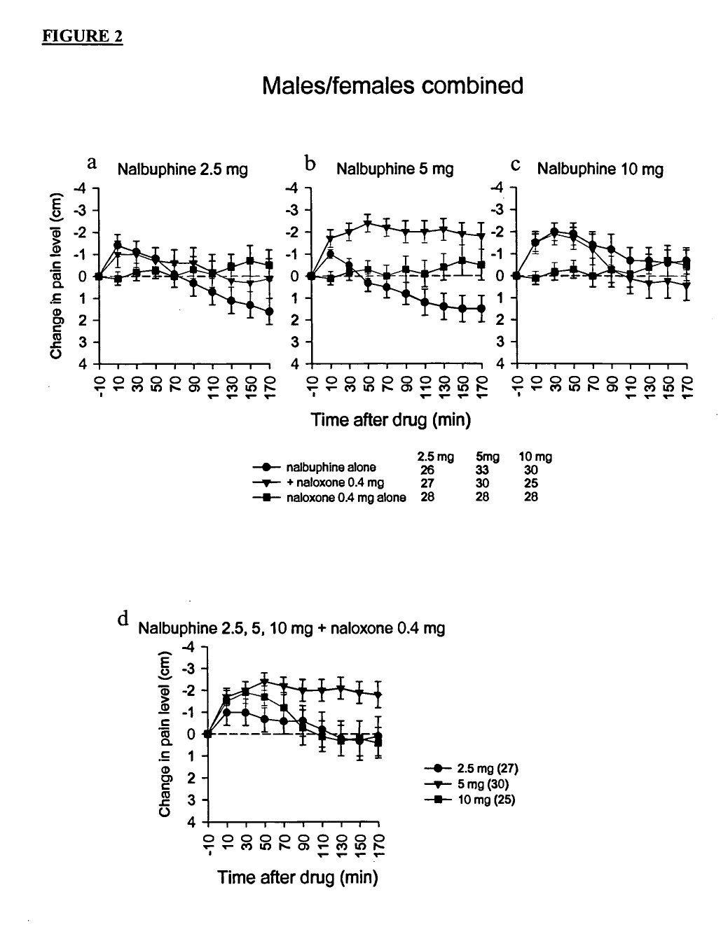

FIGS. 1a-d show comparative effects of administration of 2.5, 5 and 10 mg nalbuphine hydrochloride salt in combination with 0.4 mg of naloxone hydrochloride salt on postoperative pain. FIG. 1a illustrates that the combination of 2.5 mg nalbuphine hydrochloride salt with 0.4 mg naloxone hydrochloride salt, i.e., a ratio of 1:6.52 naloxone to nalbuphine produced more analgesia than the administration of 2.5 mg nalbuphine alone at 70 minutes after administration and produced more analgesia than administration of 0.4 mg naloxone hydrochloride salt for the first 90 minutes after administration. FIG. 1b shows that the combination of 5 mg of nalbuphine hydrochloride salt with 0.4 mg naloxone hydrochloride salt, i.e., a ratio of 1:12.5 opioid antagonist to agonist, produced more analgesia than administration of either nalbuphine hydrochloride salt or naloxone hydrochloride salt alone. FIG. 1c shows that administration of 10 mg of nalbuphine hydrochloride salt with 0.4 mg of naloxone hydrochloride salt, i.e., a ratio of 1:25 opioid antagonist to agonist, produced analgesia similar to administration of 10 mg nalbuphine hydrochloride salt alone for the first 70 minutes after administration and produced more analgesia than administration of 0.4 mg naloxone hydrochloride salt alone during the first 90 minutes after administration. FIG. 2D compares the data presented in FIGS. 2A-C for the analgesic effects of the administration of 2.5, 5, and 10 mg of nalbuphine hydrochloride salt with 0.4 mg of naloxone hydrochloride salt on post operative pain. The combination of 5 mg nalbuphine hydrochloride salt with 0.4 mg naloxone hydrochloride salt, i.e. a ratio of 1:12.5 opioid antagonist to agonist produced more analgesia than the other two combinations. The combination of 10 mg nalbuphine hydrochloride salt with 0.4 mg naloxone hydrochloride salt produced more analgesia than the combination of 2.5 mg nalbuphine hydrochloride salt with 0.4 mg naloxone hydrochloride salt for the first 90 minutes after administration and similar analgesia thereafter.

FIGS. 2a-d shows the comparative analgesic effects of administration of 2.5, 5, and 10 mg of nalbuphine hydrochloride salt with 0.4 mg of naloxone hydrochloride salt in test subjects different than those in FIGS. 1a-d. As shown in FIG. 2a, the combination of 2.5 mg nalbuphine salt with 0.4 mg naloxone salt, i.e., a ratio of 1:6.25 opioid antagonist to agonist, produced more analgesia than administration of nalbuphine hydrochloride salt alone 70 minutes after administration and produced more analgesia than administration of 0.4 mg naloxone hydrochloride salt alone for the first 110 minutes after administration. FIG. 2b illustrates that administration of 5 mg nalbuphine hydrochloride salt with 0.4 mg naloxone hydrochloride salt, i.e., a ratio of 1:12.5 opioid antagonist to agonist, produces more analgesia than administration of either nalbuphine hydrochloride salt or naloxone hydrochloride salt alone. FIG. 2c shows that the combination of 10 mg nalbuphine hydrochloride salt with 0.4 mg naloxone hydrochloride salt, i.e., a ratio of 1:25 opioid antagonist to agonist, has similar analgesic effect as administration of 10 mg nalbuphine hydrochloride salt alone for the first 70 minutes after administration and produces more analgesia than administration of 0.4 mg naloxone hydrochloride salt alone for 90 minutes after administration. FIG. 2d compares the analgesia produced by the combinations of nalbuphine hydrochloride salt and naloxone hydrochloride salt in FIGS. 2a-c. As shown in FIG. 2d, the combination of 5 mg nalbuphine hydrochloride salt with 0.4 mg naloxone hydrochloride salt, i.e. a ratio of 1:12.5 opioid antagonist to agonist produced more analgesia than the other two combinations. The combination of 10 mg nalbuphine hydrochloride salt with 0.4 mg naloxone hydrochloride salt produced more analgesia than the combination of 2.5 mg nalbuphine hydrochloride salt with 0.4 mg naloxone hydrochloride salt for the first 90 minutes after administration and similar analgesia thereafter.

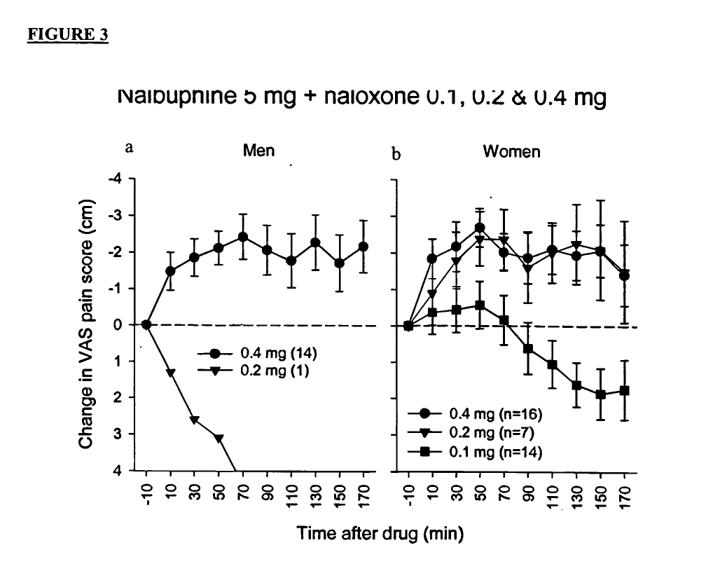

FIGS. 3a and b show the analgesic effects of administration of5 mg nalbuphine hydrochloride salt with 0.1 mg, 0.2 mg, and 0.4 mg naloxone hydrochloride salt on postoperative pain. FIG. 3a shows that in women, the combinations of 5 mg nalbuphine hydrochloride salt with 0.4 mg naloxone hydrochloride salt, i.e., a ratio of 1:12.5 opioid antagonist to agonist, and 5 mg of nalbuphine hydrochloride salt with 0.2 mg naloxone hydrochloride salt, i.e., a ratio of 1:25 opioid antagonist to agonist, produced similar amounts of analgesia, and in comparison to data presented in FIGS. 1b and 2b, more analgesia than administration of 5 mg nalbuphine hydrochloride salt alone. FIG. 3b shows inconclusive data for comparison of the analgesic effects of 5 mg nalbuphine hydrochloride salt with either 0.4 mg and 0.2 mg naloxone hydrochloride salt since only one subject was administered the combination of 5 mg nalbuphine hydrochloride salt with 0.2 mg naloxone hydrochloride salt.

### Example 2

In this clinical trial 67 patients underwent standardized surgery by the same oral surgeon for removal of third molar teeth, including at least one bony impacted mandibular third molar. Prior to surgery, patients received intravenous diazepam, nitrous oxide, and a local anesthetic (mepivacaine without vasoconstrictor to obtain a nerve block of short duration). After surgery, each patient was randomly assigned to receive an injection of nalbuphine hydrochloride salt (Abbott Laboratories, Abbott Park, Ill.) 2.5 mg either alone or combined with naloxone hydrochloride salt (0.4 mg) in an open injection, double-blinded fashion, through an intravenous line.

Criteria for administration of the test drug were an elapse of a period of at least 80 minutes after the onset of the local anesthetic and a pain rating that was greater than one quarter (2.5 cm) of the maximum possible visual analog scale (VAS) rating (10 cm). Baseline pain intensity was defined as the last VAS pain rating before administration of the test drug. VAS pain ratings were recorded at 20 minute intervals beginning ten minutes after administration of the test drug. The duration of the experiment was two hours and fifty minutes, measured from the time of administration of the test drug. For each patient, the magnitude of the analgesic (or anti-analgesic) response was defined as the difference between the pain rating at each time point following test drug administration and the baseline VAS pain rating.

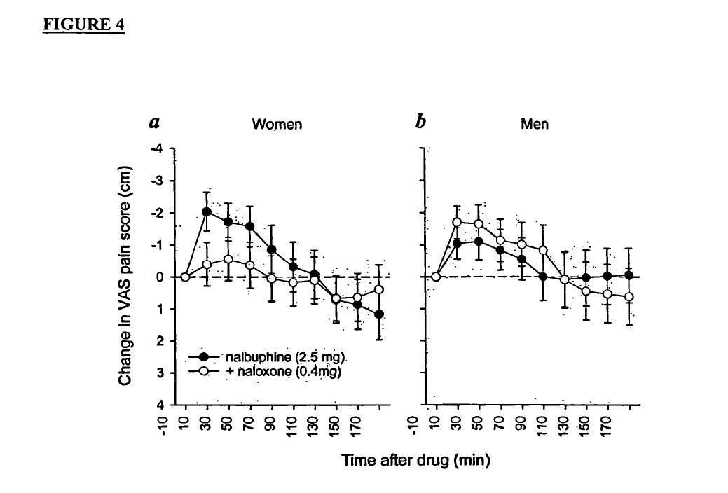

FIG. 4a depicts that in women, nalbuphine hydrochloride salt (2.5 mg) induced brief analgesia and this effect was antagonized by naloxone hydrochloride salt (0.4 g). Kappa-like opioids have been shown to act as agonists at δ-receptors, which produce dysphoria and can antagonize .t-receptor mediated antinociception in mice, and as competitive antagonists at μ-receptors. Since, in the present study, only mild analgesia was observed in the nalbuphine group and this effect was abolished by the addition of naloxone, the lower (2.5 mg) dose of nalbuphine hydrochloride salt in women is apparently insufficient to produce anti-analgesia. FIG. 4b depicts that in men, nalbuphine hydrochloride salt (2.5 mg), either alone or combined with naloxone hydrochloride salt (0.4 mg), failed to produce significant analgesia indicating that this lower dose of nalbuphine is not sufficient to induce either analgesia or anti-analgesia. Our observation that women receiving nalbuphine alone (above) experienced, mild analgesia at the early time points in the study, but that men did not, is consistent with our previous findings that 6-like opioids are more efficacious in producing analgesia in women than in men.

The present results suggest that nalbuphine: 1) acts in males and females to produce both analgesic and anti-analgesic effects, 2) the analgesic effect is greater in females and the anti-analgesic effect is greater in males, and 3) the analgesic effect occurs at a lower dose than the anti-analgesic effect. Since naloxone reverses the side effects of opioids, it is possible that optimal dosing of the partial agonist κ-opioid nalbuphine and opioid antagonist naloxone may allow the presence of a marked enhancement and prolongation of analgesia with minimal side effects.

### Example 3

In this clinical trial, 65 patients underwent standardized surgery by the same oral surgeon for removal of third molar teeth, including at least one bony impacted mandibular third molar. Prior to surgery, patients received intravenous diazepam, nitrous oxide, and a local anesthetic (mepivacaine without vasoconstrictor to obtain a nerve block of short duration). After surgery, each patient was randomly assigned to receive an injection of 2.5 mg of nalbuphine hydrochloride salt (Abbott Laboratories, Abbott Park, Ill.) either alone or combined with 0.2 mg naloxone hydrochloride salt in an open injection, double-blinded fashion, through an intravenous line.

Criteria for administration of the test drug were an elapse of a period of at least 80 minutes after the onset of the local anesthetic and a pain rating that was greater than 30% (3 cm) of the maximum possible visual analog scale (VAS) rating (10 cm). Baseline pain intensity was defined as the last VAS pain rating before administration of the test drug. VAS pain ratings were recorded at 20 minute intervals beginning ten minutes after administration of the test drug. The duration of the experiment, measured from the time of administration of the test drug, was two hours and fifty minutes. For each patient, the magnitude of the analgesic (or anti-analgesic) response was defined as the difference between the pain rating at each time point following test drug administration and the baseline VAS pain rating.

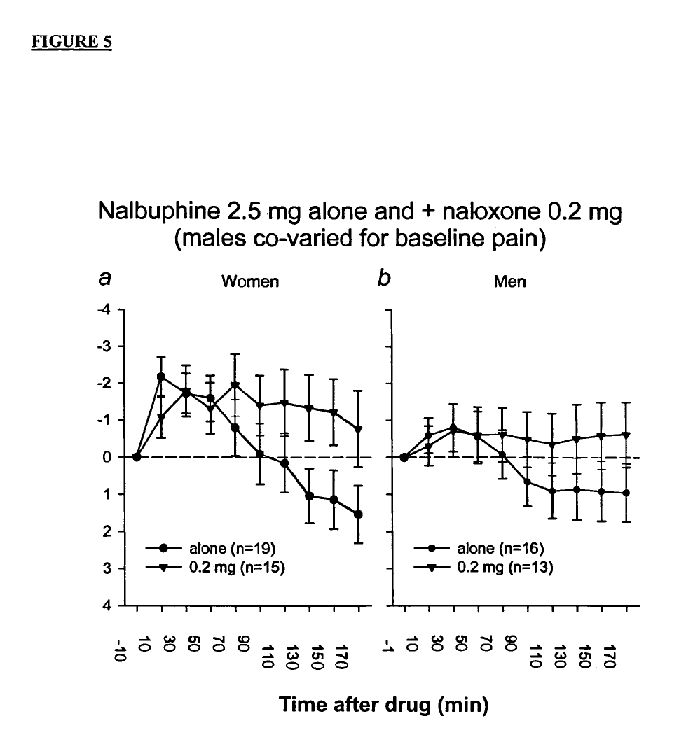

FIGS. 5a and b show that the naloxone (hydrochloride salt) to nalbuphine (hydrochloride salt) ratio of 1:12.5 (i.e. 0.2 mg to 2.5 mg) significantly enhanced the analgesic effect of nalbuphine (hydrochloride salt) (2.5 mg) in both women and men. This enhancement was manifested as a significant prolongation of the initial analgesic effect of administration of nalbuphine alone observed in both sexes.

### Example 4

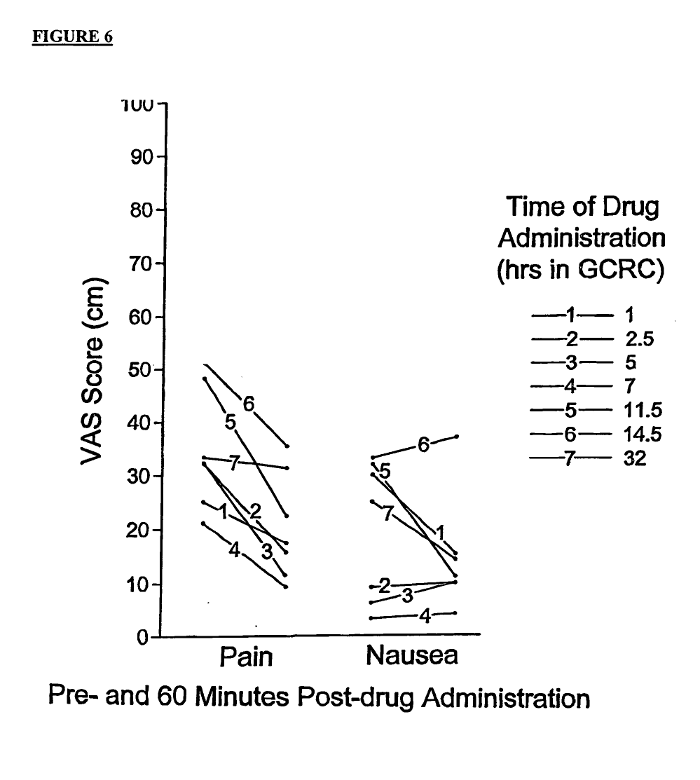

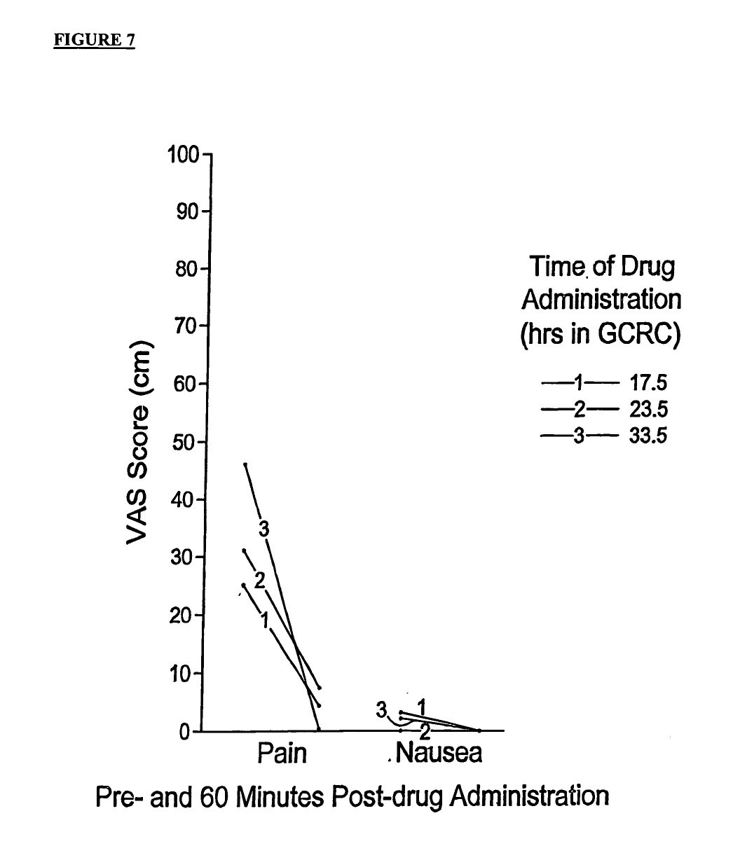

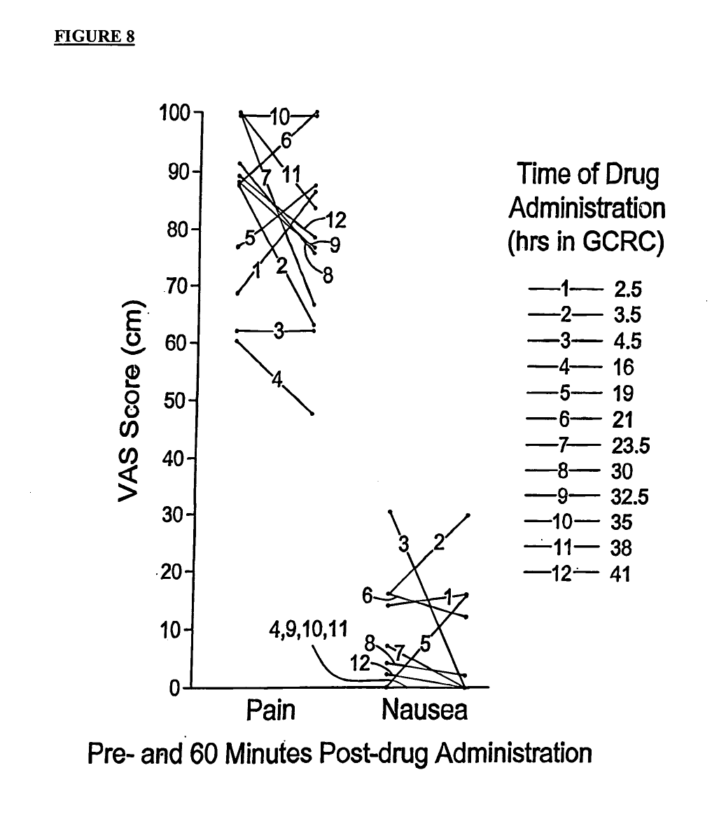

This example describes the treatment of postoperative pain following Le Fort I osteotomy using intravenous administration of a combination of nalbuphine and naloxone. In 11 all three patients, repeated administration produced marked analgesia. While a single administration of nalbuphine hydrochloride salt (5 mg) plus naloxone hydrochloride salt (0.4 mg) produces powerful and long-lasting analgesia in both males and females (Example 1, above), this analgesic could have even greater clinical impact if the efficacy were sustained dining repeated administration. In a preliminary study in three patients who underwent a Le Fort I osteotomy surgical procedure, 5 mg nalbuphine hydrochloride salt plus 0.4 mg naloxone hydrochloride salt were administered when requested by the patient. The VAS pain scores just prior to administration of the drug combination and 60 minutes afterwards are recorded in FIGS. 6, 7, and 8. The same data for the major side-effect, nausea, for these three patients, are also presented. The left end of the line represents the visual analog scale (pain and nausea) rating immediately before drug administration; the right side represents the visual analog scale rating 60 minutes after administration. As can be seen, most of the lines descend to the right, indicating that nausea as well as pain decreased following multiple administrations. Thus, three patients had repeated effective analgesia following most drug administrations over a 48-hour period, with, if anything a decrease in nausea associated with each administration. This study further suggests that early tolerance to the nalbuphine plus naloxone combination does not occur.

### Example 5

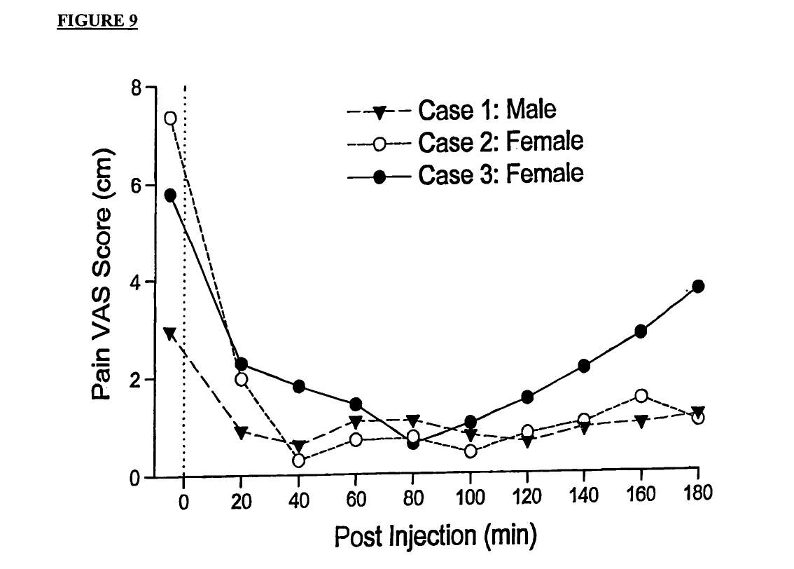

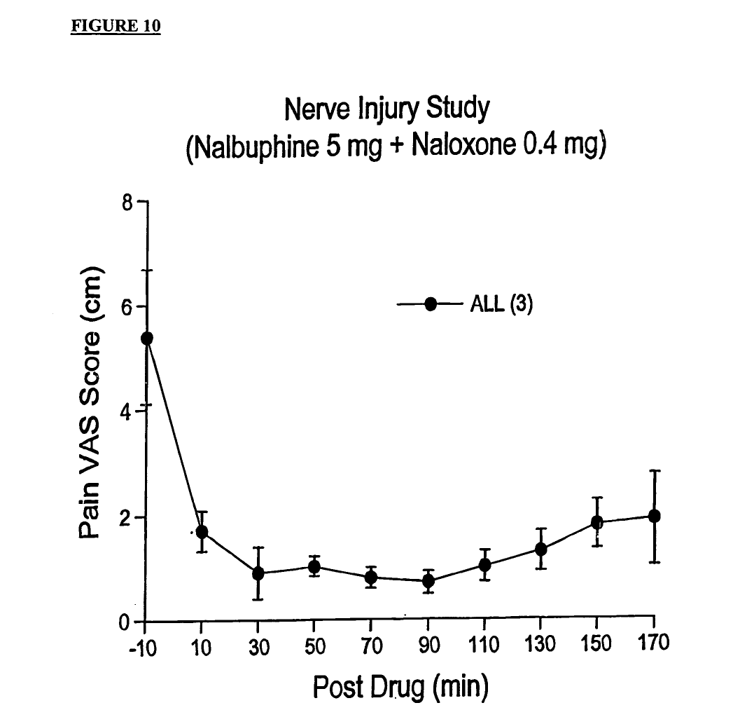

This example describes the treatment of medically refractory trigeminal neuropathy in three patients using intravenous administration of a combination of nalbuphine and naloxone. Painful peripheral neuropathies are frequent complications of chemical and mechanical injuries and metabolic disorders, and are relatively refractory to narcotic analgesics. It has been documented that inferior alveolar nerve block can cause painful peripheral neuropathy. In this example, medically refractory painful trigeminal neuropathy in three patients was treated with intravenous administration of a combination of nalbuphine hydrochloride salt (5 mg) and naloxone hydrochloride salt (0.4 mg). In all patients this combination produced marked analgesia (FIGS. 9 and 10). These findings suggest a novel form of medical management for chronic neuropathic pain. This is extremely important since neuropathic pain is poorly managed by available therapies.

In all three patients this combination produced marked analgesia. This result is believed to represent the first showing of effective treatment of neuropathic pain with a kappa-opioid.

The study involved three patients with painful peripheral neuropathy involving the mandibular division of the trigeminal nerve. One was a 42-year-old man, the second a 40 year-old woman, and the third a 25-year-old woman. Each patient discontinued all medications that had been prescribed to treat pain 2 weeks prior to the test date. They had reported that those medications had had little effect.

The test ingredients were administered via an intravenous catheter. Pain intensity was measured using a 10 cm VAS with the words “no pain” at the extreme left and the words “worst pain imaginable” at the extreme right. FIG. 9 shows the level of pain (on the VAS scale) experienced by each of the three patients individually at times up to about 180 minutes after injection. FIG. 10 is a composite for all three patients, showing the relative change in pain in the same approximate time period. Extremely marked analgesia was achieved for at least a 2.5-hour period.

### Example 6

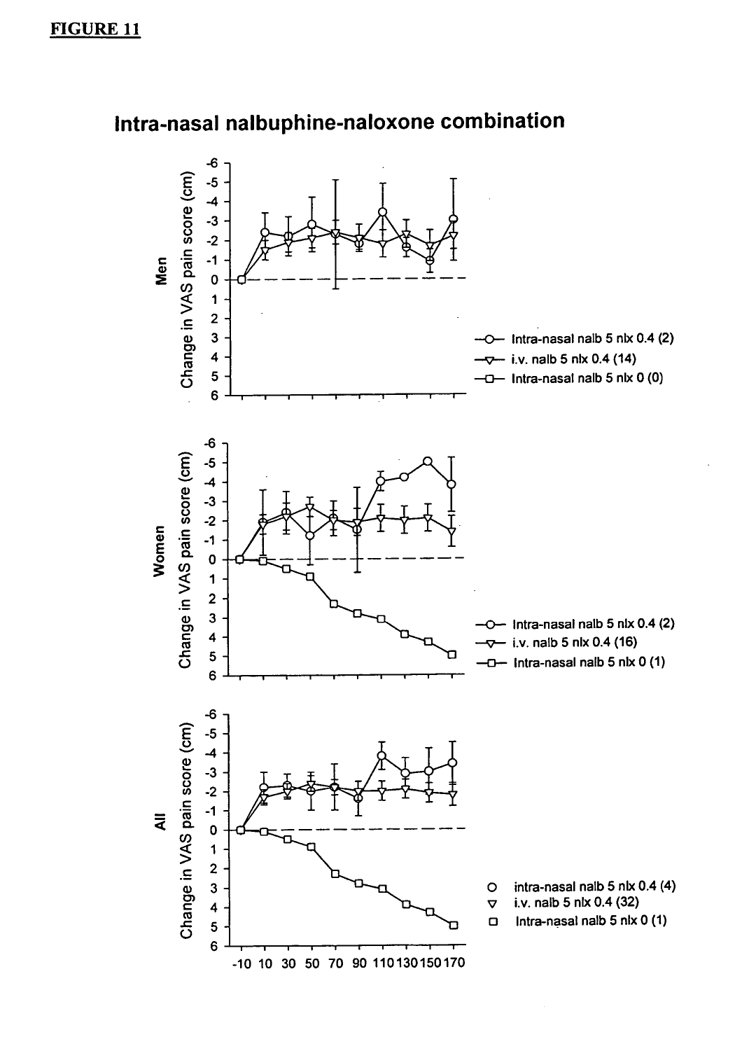

Five patients that had bony impacted mandibular third molar (wisdom) teeth removed under local anesthetic block, were studied. Following dissipation of the nerve block patients developed moderate-to-severe pain. When their pain level was ≧30 on a 10 cm visual analog scale, they patient received either nalbuphine hydrochloride salt (5 mg) plus naloxone hydrochloride salt (0.4 mg) or just the same dose of nalbuphine hydrochloride salt (5 mg), by nasal spray (Pfeiffer nasal spray units were used). The nasal spray contained, in addition to the two hydrochloride salts, citrate, citric acid, HCl and water. Pain ratings were obtained every 20 minutes, the nasal spray administration being half-way between two pain ratings. FIG. 11 shows the response to the nasal spray as change in pain, decrease in pain indicates analgesia. Also included, for comparison, is the response for 32 patients who underwent the same surgical procedure, to intravenous administration of nalbuphine hydrochloride salt (5 mg) plus naloxone hydrochloride salt (0.4 mg).

It is understood that the examples and embodiments described herein are for illustrative purposes only and that various modifications or changes in light thereof will be suggested to persons skilled in the art and are to be included within the spirit and purview of this application and scope of the appended claims.

All publications, patents, and patent applications cited herein are hereby incorporated by reference in their entirety for all purposes.

What is claimed is:

1. A method of treating pain comprising intravenously administering to an individual in need of said treatment (a) 0.1 mg to 0.8 mg of naloxone hydrochloride salt or an equivalent amount of naloxone free base, prodrug, non-hydrochloride salt, or mixture thereof and (b) an amount of nalbuphine free base, hydrochloride salt, prodrug, non-hydrochloride salt or mixture thereof that results in greater analgesia than administration of either said naloxone hydrochloride salt or said nalbuphine alone, provided that 5 mg nalbuphine free base or an equivalent amount of nalbuphine hydrochloride salt, non-hydrochloride salt, prodrug, or mixture thereof, is not intravenously administered with 0.4 mg naloxone free base or equivalent amount of naloxone hydrochloride salt, non-hydrochloride salt, prodrug, or mixture thereof.

2. The method of claim 1 wherein the amount of nalbuphine hydrochloride salt administered is 6.25 to 49 times greater, by weight, than the amount of naloxone hydrochloride salt, or an equivalent amount of nalbuphine free base, prodrug, non-chloride salt, or mixture thereof is administered.

3. The method of claim 1 wherein the amount of nalbuphine hydrochloride salt administered is 10 to 30 times greater, by weight, than the amount of naloxone hydrochloride salt, or an equivalent amount of nalbuphine free base, prodrug, non-chloride salt, or mixture thereof is administered.

4. The method of claim 1 wherein the amount of nalbuphine hydrochloride salt administered is 9 to 15 times greater, by weight, than the amount of naloxone hydrochloride salt, or an equivalent amount of nalbuphine free base, prodrug, non-chloride salt, or mixture thereof is administered.

5. The method of claim 1 wherein 1 mg to 10 mg of nalbuphine hydrochloride salt is administered, or an equivalent amount of nalbuphine free base, prodrug, non-chloride salt, or mixture thereof is administered, provided that 5 mg nalbuphine free base is not administered with 0.4 mg naloxone free base.

6. The method of claim 1 wherein 1 mg to 6 mg of nalbuphine hydrochloride salt is administered, or an equivalent amount of nalbuphine free base, prodrug, non-chloride salt, or mixture thereof is administered, provided that 5 mg nalbuphine free base is not administered with 0.4 mg naloxone free base.

7. The method of any of the claims 1 to 6 wherein 0.1 mg to 0.4 mg naloxone hydrochloride salt or an equivalent amount of naloxone free base, prodrug, non-chloride salt, or mixture thereof is administered.

8. The method of any of the claims 1 to 6 wherein 0.4 mg to 0.8 mg naloxone hydrochloride salt or an equivalent amount of naloxone free base, prodrug, non-chloride salt, or mixture thereof is administered.

9. A method of treating pain comprising administering intravenously to an individual in need of said treatment (a) 0.1 mg to 0.8 mg of naloxone hydrochloride salt or an equivalent amount of naloxone free base, prodrug, non-hydrochloride salt, or mixture thereof and (b) an amount of pentazocine free base, hydrochloride salt, prodrug, non-hydrochloride salt or mixture thereof that results in greater analgesia than administration of either said naloxone hydrochloride salt or said pentazocine hydrochloride salt alone, provided that 60 mg of pentazocine free base or an equivalent amount of pentazocine hydrochloride salt, non-hydrochloride salt, prodrug, or mixture thereof, is not administered with 0.4 mg naloxone free base or equivalent amount of naloxone hydrochloride salt, non-hydrochloride salt, prodrug, or mixture thereof.

10. The method of claim 9 wherein pentazocine hydrochloride salt is administered in an amount 18 to 120 times greater, by weight, than the amount of naloxone hydrochloride salt, or an equivalent amount of pentazocine free base, prodrug, non-hydrochloride salt, or mixture thereof is administered.

11. The method of claim 9 wherein pentazocine hydrochloride salt is administered in an amount 18 to 50 times greater, by weight, than the amount of naloxone hydrochloride salt, or an equivalent amount of pentazocine free base, prodrug, non-hydrochloride salt, or mixture thereof is administered.

12. The method of claim 9 wherein 3 mg to 50 mg pentazocine hydrochloride salt or an equivalent amount of pentazocine free base, prodrug, hydrochloride salt, or mixture thereof is administered.

13. The method of claim 9 wherein 3 mg to 30 mg pentazocine hydrochloride salt or an equivalent amount of pentazocine free base, prodrug, hydrochloride salt, or mixture thereof is administered.

14. A method of treating pain comprising administering intravenously to an individual in need of said treatment (a) 0.1 mg to 0.8 mg of naloxone hydrochloride salt or an equivalent amount of naloxone free base, prodrug, non-hydrochloride salt, or mixture thereof and (b) an amount of butorphanol free base, prodrug, pharmaceutically acceptable salt or mixture thereof that results in greater analgesia than administration of either said nalbuphine hydrochloride salt or said butorphanol alone.

15. The method of claim 14 wherein 0.3 to 10 times greater, by weight, butorphanol tartrate salt than naloxone hydrochloride salt is administered, or equivalent amount of butorphanol free base, non-tartrate salt, prodrug, or mixture thereof and/or naloxone free base, non-hydrochloride salt, prodrug, or mixture thereof is administered.

16. The method of claim 14 wherein 0.5 to 4 times greater, by weight, butorphanol tartrate salt than naloxone hydrochloride salt is administered, or equivalent amount of butorphanol free base, non-tartrate salt, prodrug, or mixture thereof and/or naloxone free base, non-hydrochloride salt, prodrug, or mixture thereof is administered.

17. The method of claim 14 wherein 0.2 mg to 2 mg butorphanol tartrate salt or an equivalent amount of butorphanol free base, non-tartrate salt, prodrug, or mixture thereof is administered.

18. The method of claim 14 wherein 0.2 mg to 1 mg butorphanol tartrate salt or an equivalent amount of butorphanol free base, non-tartrate salt, prodrug, or mixture thereof is administered.

19. A method of treating pain comprising administering, by a method other than intravenous administration, to an individual in need of said treatment (a) 0.1 mg to 0.8 mg of naloxone hydrochloride salt or an equivalent amount of naloxone free base, prodrug, non-hydrochloride salt, or mixture thereof and (b) an amount of nalbuphine free base, prodrug, pharmaceutically acceptable salt or mixture thereof that results in greater analgesia than administration of either said naloxone hydrochloride salt or said nalbuphine alone.

20. The method of claim 19 wherein the nalbuphine free base, salt, prodrug, or mixture thereof, and naloxone are administered in amounts equivalent to intravenous administration of 6.25 to 30 times greater in weight of nalbuphine hydrochloride salt than naloxone hydrochloride salt.

21. The method of claim 19 wherein the nalbuphine free base, salt, prodrug, or mixture thereof, and naloxone are administered in amounts equivalent to intravenous administration of 10 to 20 times greater in weight of nalbuphine hydrochloride salt than naloxone hydrochloride salt.

22. The method of claim 19 wherein the nalbuphine free base, salt, prodrug, or mixture thereof, is administered in an amount equivalent to intravenous administration of 1 mg to 10 mg of nalbuphine hydrochloride salt.

23. The method of claim 19 wherein the nalbuphine free base, salt, prodrug, or mixture thereof, is administered in an amount equivalent to intravenous administration of 2 mg to 8 mg of nalbuphine hydrochloride salt.

24. The method of claim 19 wherein the nalbuphine free base, salt, prodrug, or mixture thereof, is administered in an amount equivalent to intravenous administration of 1 mg to 4 mg of nalbuphine hydrochloride salt.

25. The method of claim 19 wherein the method of administration is mucosal.

26. The method of claim 25 wherein administration is nasal.

27. The method of claim 26 wherein the nasal administration is in the form of a nasal spray or nose drops.

28. The method of claim 19 wherein administration is sublingual.

29. The method of claim 28 wherein 1 to 60 times greater, by weight, nalbuphine hydrochloride salt is administered than naloxone hydrochloride salt, or equivalent amounts thereof, respectively, of nalbuphine free base, non-hydrochloride salt, prodrug, or mixture thereof and naloxone free base, non-hydrochloride salt, prodrug, and mixture thereof.

30. The method of claim 28 wherein 1 to 30 times greater, by weight, nalbuphine hydrochloride salt is administered than naloxone hydrochloride salt, or equivalent amounts thereof, respectively, of nalbuphine free base, non-hydrochloride salt, prodrug, or mixture thereof and naloxone free base, non-hydrochloride salt, prodrug, and mixture thereof.

31. The method of claim 28 wherein 5 mg to 65 mg nalbuphine hydrochloride salt or an equivalent amount of nalbuphine free base, non-hydrochloride salt, prodrug, or mixture thereof is administered.

32. The method of claim 28 wherein 7.5 mg to 30 mg nalbuphine hydrochloride salt or an equivalent amount of nalbuphine free base, non-hydrochloride salt, prodrug, or mixture thereof is administered.

33. The method of claim 28 wherein 0.4 mg to 4 mg naloxone hydrochloride salt or an equivalent amount of naloxone free base, non-hydrochloride salt, prodrug, or mixture thereof is administered.

34. The method of claim 28 wherein 0.4 mg to 2 mg naloxone hydrochloride salt or an equivalent amount of naloxone free base, non-hydrochloride salt, prodrug, or mixture thereof is administered.

35. A method of treating pain comprising administering, by a method other than intravenous administration, to an individual in need of said treatment (a) 0.1 mg to 0.8 mg of naloxone hydrochloride salt or an equivalent amount of naloxone free base, prodrug, non-hydrochloride salt, or mixture thereof and (b) an amount of pentazocine free base, hydrochloride salt, prodrug, non-hydrochloride salt or mixture thereof that results in greater analgesia than administration of either said naloxone hydrochloride salt or said pentazocine hydrochloride salt alone, provided that 50 mg of pentazocine free base or an equivalent amount of pentazocine hydrochloride salt, non-hydrochloride salt, prodrug, or mixture thereof, is not administered orally for gastro-intestinal uptake with 0.5 mg naloxone free base or equivalent amount of naloxone hydrochloride salt, non-hydrochloride salt, prodrug, or mixture thereof.

36. The method of claim 35 wherein the pentazocine free base, salt, prodrug, or mixture thereof, is administered in an amount equivalent to intravenous administration of naloxone hydrochloride salt and 18 to 120 times greater, by weight, of pentazocine hydrochloride salt.

37. The method of claim 35 wherein the pentazocine free base, salt, prodrug, or mixture thereof, is administered in an amount equivalent to intravenous administration of 10 mg to 50 mg of pentazocine hydrochloride salt.

38. The method of claim 35 wherein the pentazocine free base, salt, prodrug, or mixture thereof, is administered in an amount equivalent to intravenous administration of 15 mg to 30 mg of pentazocine hydrochloride salt.

39. The method of claim 35 wherein administration is nasal.

40. The method of claim 39 wherein administration is nasal in the form of a nasal spray or nose drops.

41. The method of claim 35 wherein administration is sublingual.

42. The method of claim 41 wherein 7.5 to 50 times greater, by weight, pentazocine hydrochloride salt is administered than naloxone hydrochloride salt, or equivalent amounts thereof, respectively, of pentazocine or naloxone free base, non-hydrochloride salt, prodrug, or mixture thereof.

43. The method of claim 41 wherein 10 to 30 times greater, by weight, pentazocine hydrochloride salt is administered than naloxone hydrochloride salt, or equivalent amounts thereof, respectively, of pentazocine or naloxone free base, non-hydrochloride salt, prodrug, or mixture thereof.

44. The method of claim 41 wherein 30 mg to 100 mg pentazocine hydrochloride salt or equivalent amount of pentazocine free base, non-hydrochloride salt, prodrug or mixture thereof is administered.

45. The method of claim 41 wherein 30 mg to 60 mg pentazocine hydrochloride salt or equivalent amount of pentazocine free base, non-hydrochloride salt, prodrug or mixture thereof is administered.

46. The method of claim 41 wherein 2 mg to 4 mg naloxone hydrochloride salt or equivalent amount of naloxone free base, non-hydrochloride salt, prodrug or mixture thereof is administered.

47. A method of treating pain comprising administering, by a method other than intravenous administration, to an individual in need of said treatment (a) 0.1 mg to 0.8 mg of naloxone hydrochloride salt or an equivalent amount of naloxone free base, prodrug, non-hydrochloride salt, or mixture thereof and (b) an amount of butorphanol free base, prodrug, pharmaceutically acceptable salt or mixture thereof that results in greater analgesia than administration of either said nalbuphine hydrochloride salt or said butorphanol alone.

48. The method of claim 47 wherein the butorphanol free base, salt, prodrug, or mixture thereof is administered in an amount equivalent to intravenous administration of naloxone hydrochloride salt and 0.3 to 10 times greater, by weight, butorphanol tartrate salt.

49. The method of claim 47 wherein naloxone and butorphanol free base, salt, prodrug, or mixture thereof is administered in an amount equivalent to intravenous administration of naloxone hydrochloride salt and 0.5 to 4 times greater, by weight, butorphanol tartrate salt than naloxone hydrochloride salt.

50. The method of claim 47 wherein butorphanol free base, salt, prodrug, or mixture thereof is administered in an amount equivalent to intravenous administration of 0.2 mg to 2 mg butorphanol tartrate salt.

51. The method of claim 47 wherein butorphanol free base, salt, prodrug, or mixture thereof is administered in an amount equivalent to intravenous administration of 0.2 mg to 1 mg butorphanol tartrate salt.

52. The method of claim 47 wherein the method of administration is mucosal.

53. The method of claim 52 wherein administration is nasal.

54. The method of claim 53 wherein administration is nasal in the form of a nasal spray or nose drops.

55. The method of claim 47 wherein administration is sublingual.

56. The method of claim 55 wherein 0.1 to 60 times greater, by weight, butorphanol tartrate salt is administered than naloxone hydrochloride salt, or equivalent amounts thereof, respectively, of butorphanol and/or naloxone free base, other salt, prodrug, or mixture thereof is administered.

57. The method of claim 55 wherein 10-30 times greater, by weight, butorphanol tartrate salt is administered than naloxone hydrochloride salt, or equivalent amounts thereof, respectively, of butorphanol and/or naloxone free base, other salt, prodrug, or mixture thereof is administered.

58. The method of claim 55 wherein 0.1 mg to 7 mg butorphanol tartrate salt or an equivalent amount of butorphanol free base, other salt, prodrug, or mixture thereof is administered.

59. The method of claim 55 wherein 0.5 mg to 6 mg butorphanol tartrate salt or an equivalent amount of butorphanol free base, other salt, prodrug, or mixture thereof is administered.

60. The method of claim 55 wherein 0.1 to 1 mg naloxone hydrochloride salt or an equivalent amount of naloxone free base, other salt, prodrug, or mixture thereof is administered.

61. A method of treating pain comprising administering intravenously to an individual in need of said treatment (a) 0.1 mg to 0.8 mg of the naloxone hydrochloride salt or an equivalent amount of naloxone free base, prodrug, non-hydrochloride salt, or mixture thereof and (b) an amount of a κ opioid agonist selected from a group consisting of benzomorphan, benzacetamide, phenothiazine, thiazine, and a benzodiazepine, or a pharmaceutically acceptable salt, prodrug, derivative, or mixture thereof, that results in greater analgesia than administration of either said naloxone hydrochloride salt or said K opioid agonist alone.

62. The method of claim 61 wherein the kappa opioid agonist is selected from the group consisting of benzomorphan, benzacetamide, phenothiazine, thiazine, and a benzodiazepine, or a pharmaceutically acceptable salt, prodrug, derivative, or mixture thereof.

63. The method of claim 62 wherein the benzomorphan derivative is selected from the group consisting of ketazocine, ethylketazocine, dezocine, bremazocine, and Tifluadom, or pharmaceutically acceptable salt, prodrug, or mixture thereof.

64. The method of claim 62 wherein the benzacetamide derivative is selected from the group consisting of U50,488, U69,593, U62,606 (spiradoline), PD 117302, CI-977, DuP 747, ICI 197067, ICI 199441, BRL 52537A, and BRL 52656A, or a pharmaceutically acceptable salt, prodrug, or mixture thereof.

65. The method of claim 62 wherein the kappa opioid agonist is the phenothiazine derivative, Rp60180, or a pharmaceutically acceptable salt, prodrug, or mixture thereof.

66. The method of claim 62 wherein the kappa receptor agonist is the thiazine derivative, R084760, or a pharmaceutically acceptable salt, prodrug, or mixture thereof.

67. A method of treating pain comprising administering to an individual in need of said treatment by a method other than intravenous administration (a) 0.1 mg to 0.8 mg of the naloxone hydrochloride salt or an equivalent amount of naloxone free base, prodrug, non-hydrochloride salt, or mixture thereof and (b) an amount of a K opioid agonist selected from a group consisting of benzomorphan, benzacetamide, phenothiazine, thiazine, and a benzodiazepine, or a pharmaceutically acceptable salt, prodrug, derivative, or mixture thereof, that results in greater analgesia than administration of either said naloxone hydrochloride salt or said κ opioid agonist alone.

68. The method of claim 67 wherein the kappa opioid agonist is selected from the group consisting of benzomorphan, benzacetamide, phenothiazine, thiazine, and a benzodiazepine, or a pharmaceutically acceptable salt, prodrug, derivative, or mixture thereof.

69. The method of claim 68 wherein the benzomorphan derivative is selected from the group consisting of ketazocine, ethylketazocine, dezocine, bremazocine, and Tifluadom, or pharmaceutically acceptable salt, prodrug, or mixture thereof.

70. The method of claim 68 wherein the benzacetamide derivative is selected from the group consisting of U50,488, U69,593, U62,606 (spiradoline), PD 117302, CI-977, DuP 747, ICI 197067, ICI 199441, BRL 52537A, and BRL 52656A, or a pharmaceutically acceptable salt, prodrug, or mixture thereof.

71. The method of claim 68 wherein the kappa opioid agonist is the phenothiazine derivative, Rp60180, or a pharmaceutically acceptable salt, prodrug, or mixture thereof.

72. The method of claim 68 wherein the kappa receptor agonist is the thiazine derivative, R084760, or a pharmaceutically acceptable salt, prodrug, or mixture thereof.

73. A pharmaceutical composition comprising (a) 0.1 mg to 0.8 mg of naloxone hydrochloride salt or an equivalent amount of naloxone freebase, prodrug, non-hydrochloride salt, or mixture thereof; (b) an amount of nalbuphine free base, salt, prodrug, or mixture thereof that results in greater analgesia than administration of said nalbuphine or said naloxone alone; and (c) a pharmaceutically acceptable carrier, said pharmaceutical composition being formulated for intravenous administration, provided that the composition does not comprise 5 mg nalbuphine free base with 0.4 mg naloxone free base.

74. The composition of claim 73 wherein the amount of nalbuphine hydrochloride salt is 6.25 to 49 times greater, by weight, than the amount of naloxone hydrochloride salt, or an equivalent amount of nalbuphine free base, prodrug, non-chloride salt, or mixture thereof.

75. The composition of claim 73 wherein the amount of nalbuphine hydrochloride salt is 8 to 30 times greater, by weight, than the amount of naloxone hydrochloride salt, or an equivalent amount of nalbuphine free base, prodrug, non-chloride salt, or mixture thereof.

76. The composition of claim 73 wherein the amount of nalbuphine hydrochloride salt is 10 to 20 times greater, by weight, than the amount of naloxone hydrochloride salt, or an equivalent amount of nalbuphine free base, prodrug, non-chloride salt, or mixture thereof.

77. The composition of claim 73 wherein the amount of nalbuphine hydrochloride salt is 1 mg to 10 mg or an equivalent amount of nalbuphine free base, prodrug, non-chloride salt, or mixture thereof.

78. The composition of claim 73 wherein the amount of nalbuphine hydrochloride salt is 3 mg to 8 mg or an equivalent amount of nalbuphine free base, prodrug, non-chloride salt, or mixture thereof.

79. The composition of claim 73 wherein the amount of nalbuphine hydrochloride salt is 1 mg to 6 mg or an equivalent amount of nalbuphine free base, prodrug, non-chloride salt, or mixture thereof.

80. The composition of claim 73 comprising 0.5 mg to 0.8 mg naloxone hydrochloride salt or an equivalent amount of naloxone free base, prodrug, non-chloride salt, or mixture thereof.

81. The composition of claim 73 comprising 0.1 mg to 0.5 mg naloxone hydrochloride salt or an equivalent amount of naloxone free base, prodrug, non-chloride salt, or mixture thereof is administered, provided that the composition does not comprise 5 mg nalbuphine free base with 0.4 mg naloxone free base.

82. A pharmaceutical composition comprising (a) 0.1 mg to 0.8 mg of naloxone hydrochloride salt or an equivalent amount of naloxone freebase, prodrug, non-hydrochloride salt, or mixture thereof; (b) an amount of pentazocine free base, salt, prodrug, or mixture thereof that results in greater analgesia than administration of said pentazocine or said naloxone hydrochloride salt alone; and (c) a pharmaceutically acceptable carrier, said pharmaceutical composition being formulated for intravenous administration, provided that the composition does not comprise 60 mg of pentazocine with 0.4 mg naloxone.

83. The composition of claim 82 comprising pentazocine hydrochloride salt is administered in an amount 18 to 120 times greater, by weight, than the amount of naloxone hydrochloride salt or an equivalent amount of pentazocine free base, prodrug, non-hydrochloride salt, or mixture thereof, provided that the composition does not comprise 60 mg of pentazocine with 0.4 mg naloxone.

84. The composition of claim 82 comprising pentazocine hydrochloride salt is administered in an amount 18 to 50 times greater, by weight, than the amount of naloxone hydrochloride salt or an equivalent amount of pentazocine free base, prodrug, non-hydrochloride salt, or mixture thereof, provided that the composition does not comprise 60 mg of pentazocine with 0.4 mg naloxone.

85. The composition of claim 82 comprising 3 mg to 50 mg pentazocine hydrochloride salt or an equivalent amount of pentazocine free base, prodrug, hydrochloride salt, or mixture thereof.

86. The composition of claim 82 comprising 3 mg to 30 mg pentazocine hydrochloride salt or an equivalent amount of pentazocine free base, prodrug, hydrochloride salt, or mixture thereof.

87. The composition of claim 82 comprising 10 mg to 20 mg pentazocine hydrochloride salt or an equivalent amount of pentazocine free base, prodrug, hydrochloride salt, or mixture thereof.

88. A pharmaceutical composition comprising (a) 0.1 mg to 0.8 mg of naloxone hydrochloride salt or an equivalent amount of naloxone freebase, prodrug, non-hydrochloride salt, or mixture thereof; (b) an amount of butorphanol free base, salt, prodrug, or mixture thereof that results in greater analgesia than administration of said butorphanol or said naloxone alone; and (c) a pharmaceutically acceptable carrier, said pharmaceutical composition being formulated for intravenous administration.

89. The composition of claim 88 comprising 0.3 to 10 times greater, by weight, butorphanol tartrate salt than naloxone hydrochloride salt, or equivalent amounts of, respectively, butorphanol free base, non-tartrate salt, prodrug, or mixture thereof and naloxone free base, non-hydrochloride salt, prodrug, or mixture thereof.

90. The composition of claim 88 comprising 0.3 to 6 times greater, by weight, butorphanol tartrate salt than naloxone hydrochloride salt, or equivalent amounts of, respectively, butorphanol free base, non-tartrate salt, prodrug, or mixture thereof and naloxone free base, non-hydrochloride salt, prodrug, or mixture thereof.

91. The composition of claim 88 comprising 0.2 mg to 2 mg butorphanol tartrate salt or an equivalent amount of butorphanol free base, non-tartrate salt, prodrug, or mixture thereof.

92. The composition of claim 88 comprising 0.2 mg to 1.2 mg butorphanol tartrate salt or an equivalent amount of butorphanol free base, non-tartrate salt, prodrug, or mixture thereof.

93. A pharmaceutical composition comprising (a) 0.1 mg to 0.8 mg of naloxone hydrochloride salt or an equivalent amount of naloxone freebase, prodrug, non-hydrochloride salt, or mixture thereof; (b) an amount of a κ opioid agonist selected from the group consisting of benzomorphan, benzacetamide, phenothiazine, thiazine, and a benzodiazepine, or a pharmaceutically acceptable salt, prodrug, derivative, or mixture thereof, that results in greater analgesia than administration of said κ-opioid agonist or said naloxone alone; and (c) a pharmaceutically acceptable carrier, said pharmaceutical composition being formulated for intravenous administration.

94. The composition of claim 93 wherein the kappa opioid agonist is selected from the group consisting of benzomorphan, benzacetamide, phenothiazine, thiazine, and a benzodiazepine, or a pharmaceutically acceptable salt, prodrug, derivative, or mixture thereof.

95. The composition of claim 93 wherein the benzodiazepine derivative is selected from the group consisting of ketazocine, ethylketazocine, dezocine, bremazocine, and Tifluadom, or pharmaceutically acceptable salt, prodrug, or mixture thereof.

96. The composition of claim 93 wherein the benzacetamide derivative is selected from the group consisting of U50,488, U69,593, U62,606 (spiradoline), PD 117302, CI-977, DuP 747, ICI 197067, ICI 199441, BRL 52537A, and BRL 52656A, or a pharmaceutically acceptable salt, prodrug, or mixture thereof.

97. The composition of claim 93 wherein the kappa opioid agonist is the phenothiazine derivative, Rp60180, or a pharmaceutically acceptable salt, prodrug, or mixture thereof.

98. The composition of claim 93 wherein the kappa receptor agonist is the thiazine derivative, R084760, or a pharmaceutically acceptable salt, prodrug, or mixture thereof.

99. The composition of claim 93 wherein the kappa opioid agonist is nalbuphine or a hydrochloride salt, non-hydrochloride salt, prodrug, or mixture thereof.

100. A pharmaceutical composition comprising (a) 0.1 mg to 0.8 mg of an naloxone hydrochloride salt or an equivalent amount of naloxone freebase, prodrug, non-hydrochloride salt, or mixture thereof; (b) an amount of nalbuphine free base, salt, prodrug, or mixture thereof that results in greater analgesia than administration of the said nalbuphine or said naloxone alone; and (c) a pharmaceutically acceptable carrier, said pharmaceutical composition being formulated for administration other than intravenous administration.

101. The composition claim 100 comprising nalbuphine free base, salt, prodrug, or mixture thereof, in an amount equivalent to intravenous administration of the naloxone hydrochloride salt and 6.25 to 30 times greater in weight of nalbuphine hydrochloride salt than the naloxone hydrochloride salt.

102. The composition claim 100 comprising nalbuphine free base, salt, prodrug, or mixture thereof, in an amount equivalent to intravenous administration of the naloxone hydrochloride salt and 10 to 20 times greater in weight of nalbuphine hydrochloride salt than the naloxone hydrochloride salt.

103. The composition claim 100 comprising nalbuphine free base, salt, prodrug, or mixture thereof, in an amount equivalent to intravenous administration of 1 mg to 10 mg of nalbuphine hydrochloride salt.

104. The composition claim 100 comprising nalbuphine free base, salt, prodrug, or mixture thereof, in an amount equivalent to intravenous administration of 1 mg to 6 mg of nalbuphine hydrochloride salt.

105. The composition of any of the claims 100 to 104 wherein the composition is formulated for mucosal administration.

106. The composition of claim 105 wherein the composition is formulated for nasal administration.

107. The composition of claim 105 wherein the composition is formulated for nasal administration in the form of a nasal spray or nose drops.

108. The composition of claims 100 wherein the composition is formulated for sublingual administration.

109. The composition of claim 108 comprising 1 to 60 times greater, by weight, nalbuphine hydrochloride salt than naloxone hydrochloride salt, or equivalent amounts thereof, respectively, of nalbuphine free base, non-hydrochloride salt, prodrug, or mixture thereof and naloxone free base, non-hydrochloride salt, prodrug, and mixture thereof.

110. The composition of claim 108 comprising 1 to 30 times greater, by weight, nalbuphine hydrochloride salt is administered than naloxone hydrochloride salt, or equivalent amounts thereof, respectively, of nalbuphine free base, non-hydrochloride salt, prodrug, or mixture thereof and naloxone free base, non-hydrochloride salt, prodrug, and mixture thereof.

111. The composition of claim 108 comprising 5 mg to 65 mg nalbuphine hydrochloride salt or an equivalent amount of nalbuphine free base, non-hydrochloride salt, prodrug, or mixture thereof.

112. The composition of claim 108 comprising 7.5 mg to 30 mg nalbuphine hydrochloride salt or an equivalent amount of nalbuphine free base, non-hydrochloride salt, prodrug, or mixture thereof.

113. The composition of any claims 108 to 112 comprising 0.4 mg to 4 mg naloxone hydrochloride salt or an equivalent amount of naloxone free base, non-hydrochloride salt, prodrug, or mixture thereof.

114. The composition of any claims 108 to 112 comprising 0.4 mg to 2.5 mg naloxone hydrochloride salt or an equivalent amount of naloxone free base, non-hydrochloride salt, prodrug, or mixture thereof.

115. A pharmaceutical composition comprising (a) 0.1 mg to 0.8 mg of naloxone hydrochloride salt or an equivalent amount of naloxone freebase, prodrug, non-hydrochloride salt, or mixture thereof; (b) an amount of pentazocine free base, salt, prodrug, or mixture thereof that results in greater analgesia than administration of said pentazocine or said naloxone alone; and (c) a pharmaceutically acceptable carrier, said pharmaceutical composition being formulated for administration other than intravenous administration, provided that the composition formulated for oral administration does not comprise 50 mg pentazocine hydrochloride salt with 0.5 mg naloxone hydrochloride salt.

116. The composition of claim 115 comprising an amount of pentazocine free base, salt, prodrug, or mixture thereof, equivalent to intravenous administration of the opioid antagonist hydrochloride salt and 18 to 120 times greater, by weight, of pentazocine hydrochloride salt.

117. The composition of claim 115 comprising an amount of pentazocine free base, salt, prodrug, or mixture thereof, equivalent to intravenous administration of 10 mg to 50 mg of pentazocine hydrochloride salt.

118. The composition of claim 115 comprising an amount of pentazocine free base, salt, prodrug, or mixture thereof, equivalent to intravenous administration of 15 mg to 30 mg of pentazocine hydrochloride salt.

119. The composition of claim 115 wherein the composition is formulated for mucosal administration.

120. The composition of claim 119 wherein the composition is formulated for nasal administration.

121. The composition of claim 120 wherein the composition is formulated for nasal administration in the form of a nasal spray or nose drops.

122. The composition of claim 115 wherein the composition is formulated for sublingual administration.

123. The composition of claim 122 comprising 7.5 to 50 times greater, by weight, pentazocine hydrochloride salt than naloxone hydrochloride salt, or equivalent amounts thereof, respectively, of pentazocine or naloxone free base, non-hydrochloride salt, prodrug, or mixture thereof.

124. The composition of claim 122 comprising 10 to 30 times greater, by weight, pentazocine hydrochloride salt than naloxone hydrochloride salt, or equivalent amounts thereof, respectively, of pentazocine or naloxone free base, non-hydrochloride salt, prodrug, or mixture thereof.

125. The composition of claim 122 comprising 30 mg to 100 mg pentazocine hydrochloride salt or equivalent amount of pentazocine free base, non-hydrochloride salt, prodrug or mixture thereof.

126. The composition of claim 122 comprising 30 mg to 60 mg pentazocine hydrochloride salt or equivalent amount of pentazocine free base, non-hydrochloride salt, prodrug or mixture thereof.

127. The composition of claim 122 comprising 2 mg to 4 mg naloxone hydrochloride salt or equivalent amount of naloxone free base, non-hydrochloride salt, prodrug or mixture thereof.

128. A pharmaceutical composition comprising (a) 0.1 mg to 0.8 mg naloxone hydrochloride salt or an equivalent amount of naloxone freebase, prodrug, non-hydrochloride salt, or mixture thereof; (b) an amount of butorphanol free base, salt, prodrug, or mixture thereof that results in greater analgesia than administration of said butorphanol or said naloxone alone; and (c) a pharmaceutically acceptable carrier, said pharmaceutical composition being formulated for administration other than intravenous administration.

129. The composition of claim 128 comprising butorphanol free base, salt, prodrug, or mixture thereof in an amount equivalent to intravenous administration of naloxone hydrochloride salt and 0.3 to 10 times greater, by weight, butorphanol tartrate salt.

130. The composition of claim 128 comprising butorphanol free base, salt, prodrug, or mixture thereof in an amount equivalent to intravenous administration of naloxone hydrochloride salt and 0.3 to 6 times greater, by weight, butorphanol tartrate salt.

131. The composition of claim 128 comprising butorphanol free base, salt, prodrug, or mixture thereof in an amount equivalent to intravenous administration of 0.2 mg to 2 mg butorphanol tartrate salt.

132. The composition of claim 128 comprising butorphanol free base, salt, prodrug, or mixture thereof in an amount equivalent to intravenous administration of 0.2 mg to 1 mg butorphanol tartrate salt.

133. The compositions of claim 128 wherein the composition is formulated for mucosal administration.

134. The composition of claim 133 wherein the composition is formulated for nasal administration.

135. The composition of claim 134 wherein the composition is formulated for nasal administration the form of a nasal spray or nose drops.

136. The composition of claim 128 wherein the composition is formulated for sublingual administration.

137. The composition of claim 136 comprising 0.1 to 60 times greater, by weight, butorphanol tartrate salt than naloxone hydrochloride salt, or equivalent amounts thereof, respectively, of butorphanol and/or naloxone free base, other salt, prodrug, or mixture thereof.

138. The composition of claim 136 comprising 10-30 times greater, by weight, butorphanol tartrate salt is administered than naloxone hydrochloride salt, or equivalent amounts thereof, respectively, of butorphanol and/or naloxone free base, other salt, prodrug, or mixture thereof.

139. The composition of claim 138 comprising 0.1 mg to 7 mg butorphanol tartrate salt or an equivalent amount of butorphanol free base, other salt, prodrug, or mixture thereof.

140. The composition of claim 138 comprising 0.5 mg to 6 mg butorphanol tartrate salt or an equivalent amount of butorphanol free base, other salt, prodrug, or mixture thereof.

141. A pharmaceutical composition comprising (a) 0.1 mg to 0.8 mg of naloxone hydrochloride salt or an equivalent amount of naloxone freebase, prodrug, non-hydrochloride salt, or mixture thereof; (b) an amount of a κ opioid agonist selected from the group consisting of benzomorphan, benzacetamide, phenothiazine, thiazine, and a benzodiazepine, or a pharmaceutically acceptable salt, prodrug, derivative, or mixture thereof, that results in greater analgesia than administration of said κ opioid agonist or said naloxone alone; and (c) a pharmaceutically acceptable carrier, said pharmaceutical composition being formulated for intravenous administration.

142. The method of claim 141 wherein the kappa opioid agonist is selected from the group consisting of benzomorphan, benzacetamide, phenothiazine, thiazine, and a benzodiazepine, or a pharmaceutically acceptable salt, prodrug, derivative, or mixture thereof.

143. The composition of claim 142 wherein the benzodiazepine derivative is selected from the group consisting of ketazocine, ethylketazocine, dezocine, bremazocine, and Tifluadom, or pharmaceutically acceptable salt, prodrug, or mixture thereof.

144. The composition of claim 142 wherein the benzacetamide derivative is selected from the group consisting of U50,488, U69,593, U62,606 (spiradoline), PD 117302, CI-977, DuP 747, ICI 197067, ICI 199441, BRL 52537A, and BRL 52656A, or a pharmaceutically acceptable salt, prodrug, or mixture thereof.

145. The composition of claim 142 wherein the kappa opioid agonist is the phenothiazine derivative, Rp60180, or a pharmaceutically acceptable salt, prodrug, or mixture thereof.

146. The composition of claim 142 wherein the kappa receptor agonist is the thiazine derivative, R084760, or a pharmaceutically acceptable salt, prodrug, or mixture thereof.

147. The composition of claim 142 wherein the kappa opioid agonist is nalbuphine or a hydrochloride salt, non-hydrochloride salt, prodrug, or mixture thereof.

148. A pharmaceutical composition comprising (a) 0.1 mg to 0.8 mg of naloxone hydrochloride salt or an equivalent amount of naloxone freebase, prodrug, non-hydrochloride salt, or mixture thereof; (b) an amount of a K opioid agonist selected from the group consisting of benzomorphan, benzacetamide, phenothiazine, thiazine, and a benzodiazepine, or a pharmaceutically acceptable salt, prodrug, derivative, or mixture thereof, that results in greater analgesia than administration of said κ opioid agonist or said naloxone alone; and (c) a pharmaceutically acceptable carrier, said pharmaceutical composition being formulated for administration by a method other than intravenous administration.

149. The method of claim 148 wherein the kappa opioid agonist is selected from the group consisting of benzomorphan, benzacetamide, phenothiazine, thiazine, and a benzodiazepine, or a pharmaceutically acceptable salt, prodrug, derivative, or mixture thereof.

150. The composition of claim 149 wherein the benzodiazepine derivative is selected from the group consisting of ketazocine, ethylketazocine, dezocine, bremazocine, and Tifluadom, or pharmaceutically acceptable salt, prodrug, or mixture thereof.

151. The composition of claim 149 wherein the benzacetamide derivative is selected from the group consisting of U50,488, U69,593, U62,606 (spiradoline), PD 117302, CI-977, DuP 747, ICI 197067, ICI 199441, BRL 52537A, and BRL 52656A, or a pharmaceutically acceptable salt, prodrug, or mixture thereof.

152. The composition of claim 149 wherein the kappa opioid agonist is the phenothiazine derivative, Rp60180, or a pharmaceutically acceptable salt, prodrug, or mixture thereof.

153. The composition of claim 149 wherein the kappa receptor agonist is the thiazine derivative, R084760, or a pharmaceutically acceptable salt, prodrug, or mixture thereof.

154. A method of treating pain comprising mucosally administering to an individual in need of said treatment (a) 0.1 mg to 0.8 mg of a hydrochloride salt of an opioid antagonist or an equivalent amount of opioid antagonist free base, prodrug, non-hydrochloride salt, or mixture thereof, wherein said opioid antagonist is naloxone, naltrexone, methylnaltrexone, nalmefene, nalorphine, levalorphan, oxylorphan or cyprenorphine, and (b) an amount of nalbuphine free base, hydrochloride salt, prodrug, non-hydrochloride salt or mixture thereof that results in greater analgesia than administration of either said opioid antagonist hydrochloride salt or said nalbuphine alone.

155. A method according to claim 154 wherein the opioid antagonist is a salt of naloxone, naltrexone, methylnaltrexone, nalmefene, nalorphine, levalorphan, oxylorphan, or cyprenorphine.

156. A method according to claim 154 wherein the opioid antagonist is naloxone or a salt or prodrug of naloxone.

157. A method according to claim 154 wherein an opioid antagonist hydrochloride salt is administered and the amount of nalbuphine hydrochloride salt administered is 6.25 to 49 times greater, by weight, than the amount of opioid antagonist hydrochloride salt, or an equivalent amount of nalbuphine free base, prodrug, non-chloride salt, or mixture thereof, is administered.

158. A method according to claim 154 wherein an opioid antagonist hydrochloride salt is administered and the amount of nalbuphine hydrochloride salt administered is 10 to 15 times greater, by weight, than the amount of naloxone hydrochloride salt, or an equivalent amount of nalbuphine free base, prodrug, non-chloride salt, or mixture thereof, is administered.

159. A method according to claim 154 wherein an opioid antagonist hydrochloride salt is administered and the amount of nalbuphine hydrochloride salt administered is 12.5 times greater, by weight, than the amount of naloxone hydrochloride salt, or an equivalent amount of nalbuphine free base, prodrug, non-chloride salt, or mixture thereof, is administered.

160. A method according to claim 154 wherein 2 mg to 10 mg of nalbuphine hydrochloride salt is administered, or an equivalent amount of nalbuphine free base, prodrug, non-chloride salt, or mixture thereof, is administered.

161. A method according to claim 154 wherein 0.1 mg to 0.8 mg of naloxone hydrochloride salt is administered, or an equivalent amount of naloxone free base, prodrug, non-chloride salt, or mixture thereof, is administered.

162. A method according to claim 154 wherein 2 mg to 5 mg of nalbuphine hydrochloride salt is administered, or an equivalent amount of nalbuphine free base, prodrug, non-chloride salt, or mixture thereof, is administered.

163. A method according to claim 154 wherein 0.4 mg of naloxone hydrochloride and 5 mg of nalbuphine hydrochloride are administered.

164. A method according to claim 154 wherein 0.2 mg of naloxone hydrochloride and 2.5 mg of nalbuphine hydrochloride are administered.

165. A method according to claim 154 wherein 0.8 mg of naloxone hydrochloride and 10 mg of nalbuphine hydrochloride are administered.

166. A method according to claim 154 wherein the ingredients are administered intranasally.

167. A method according to claim 154 wherein the ingredients are administered by pulmonary administration.

168. A method of treating pain comprising administering to an individual in need of said treatment (a) 0.1 mg to 0.8 mg of a hydrochloride salt of an opioid antagonist or an equivalent amount of opioid antagonist free base, prodrug, non-hydrochloride salt, or mixture thereof, wherein said opioid antagonist is naloxone, naltrexone, methylnaltrexone, nalmefene, nalorphine, levalorphan, oxylorphan or cyprenorphine, and (b) an amount of a free base, hydrochloride salt, prodrug, non-hydrochloride salt or mixture thereof of a kappa-opioid, that results in greater analgesia than administration of either said opioid antagonist hydrochloride salt or said kappa-opioid, wherein the kappa-opioid is pentazocine, butorphanol, ketazocine, ethylketazocine, dezocine, bremazocine, a benzacetamide derivative, a phenothiazine derivative, a thiazine derivative, or a benzodiazepine derivative, provided that 60 mg of pentazocine free base or an equivalent amount of pentazocine hydrochloride salt, non-hydrochloride salt, prodrug, or mixture thereof, is not administered with 0.4 mg naloxone free base or equivalent amount of naloxone hydrochloride salt, non-hydrochloride salt, prodrug, or mixture thereof.

169. A method according to claim 168 wherein the kappa-opioid is pentazocine and the opioid antagonist is naloxone.

170. A method according to claim 169 wherein pentazocine hydrochloride salt is administered in an amount 18 to 30 times greater, by weight, than the amount of naloxone hydrochloride salt, or an equivalent amount of pentazocine free base, prodrug, non-hydrochloride salt, or mixture thereof is administered.

171. A method according to claim 168 wherein the kappa-opioid is butorphanol and the opioid antagonist is naloxone.

172. A method according to claim 175 wherein 0.3 to 10 times greater, by weight, butorphanol tartrate salt than naloxone hydrochloride salt is administered, or equivalent amount of butorphanol free base, non-tartrate salt, prodrug, or mixture thereof and/or naloxone free base, non-hydrochloride salt, prodrug, or mixture thereof is administered.

173. A method according to claim 168 wherein 0.1 to 0.8 mg naloxone hydrochloride salt or an equivalent amount of naloxone free base, non-hydrochloride salt, prodrug, or mixture thereof is administered

174. A method according to claim 168 wherein the ingredients are administered intravenously.

175. A method according to any of claim 168 wherein the ingredients are administered mucosally.

176. A method according to claim 168 wherein the ingredients are administered intranasally.

177. A method according to claim 168 wherein the ingredients are administered by pulmonary administration.

178. A method of treating pain comprising administering to an individual in need of said treatment (a) from 0.1 to 0.8 mg of a hydrochloride salt of naloxone or an equivalent amount of naloxone free base, prodrug, non-hydrochloride salt, or mixture thereof; and (b) from 1 to 2.5 mg of nalbuphine hydrochloride, or an equivalent amount of nalbuphine free base, prodrug, non-hydrochloride salt or mixture thereof.

179. A method of treating pain comprising administering to an individual in need of said treatment (a) from 0.1 to 0.8 mg of a hydrochloride salt of naloxone or an equivalent amount of naloxone free base, prodrug, non-hydrochloride salt, or mixture thereof; and (b) from 8.5 to 10 mg of nalbuphine hydrochloride, or an equivalent amount of nalbuphine free base, prodrug, non-hydrochloride salt or mixture thereof.

180. A method of treating pain comprising administering to an individual in need of said treatment (a) 0.1 mg of a hydrochloride salt of naloxone or an equivalent amount of naloxone free base, prodrug, non-hydrochloride salt, or mixture thereof; and (b) 1.25 mg of nalbuphine hydrochloride, or an equivalent amount of nalbuphine free base, prodrug, non-hydrochloride salt or mixture thereof.

181. A method of treating pain comprising administering to an individual in need of said treatment (a) 0.2 mg of a hydrochloride salt of naloxone or an equivalent amount of naloxone free base, prodrug, non-hydrochloride salt, or mixture thereof; and (b) 2.5 mg of nalbuphine hydrochloride, or an equivalent amount of nalbuphine free base, prodrug, non-hydrochloride salt or mixture thereof.

182. A method of treating pain comprising administering to an individual in need of said treatment (a) 0.8 mg of a hydrochloride salt of naloxone or an equivalent amount of naloxone free base, prodrug, non-hydrochloride salt, or mixture thereof; and (b) 10 mg of nalbuphine hydrochloride, or an equivalent amount of nalbuphine free base, prodrug, non-hydrochloride salt or mixture thereof.

183. A method according to claim 178 wherein the ingredients are administered intravenously.

184. A method according to claim 178 wherein the ingredients are administered mucosally.

185. A method according to claim 178 wherein the ingredients are administered intranasally.

186. A method according to claim 178 wherein the ingredients are administered by pulmonary administration.

187. A method according to claim 154 wherein the pain is neuropathic pain.

188. A method according to claim 154 wherein the pain is inflammatory pain.

189. A method according to claim 154 wherein the pain is acute pain.

190. A method according to claim 154 wherein the pain is traumatic pain.

191. A method according to claim 154 wherein the pain is post-procedural pain.

192. A method according to claim 154 wherein the pain is infection-related pain.

193. A pharmaceutical composition comprising (a) 0.1 mg to 0.8 mg of a hydrochloride salt of an opioid antagonist or an equivalent amount of opioid antagonist free base, prodrug, non-hydrochloride salt, or mixture thereof, wherein said opioid antagonist is naloxone, naltrexone, methylnaltrexone, nalmefene, nalorphine, levalorphan, oxylorphan or cyprenorphine; (b) an amount of nalbuphine free base, nalbuphine salt, nalbuphine prodrug, or mixture thereof that results in greater analgesia than administration of the nalbuphine ingredient or the opioid antagonist ingredient alone; and (c) a pharmaceutically acceptable carrier, said pharmaceutical composition being formulated for mucosal administration.

194. A composition according to claim 193 wherein the opioid antagonist is a salt of naloxone, naltrexone, methylnaltrexone, nalmefene, nalorphine, levalorphan, oxylorphan, or cyprenorphine.

195. A composition according to claim 193 wherein the opioid antagonist is naloxone or a salt or prodrug of naloxone.

196. A composition according to claim 193 comprising nalbuphine hydrochloride, the amount of nalbuphine hydrochloride salt being 6.25 to 49 times greater, by weight, than the amount of opioid antagonist hydrochloride salt, or an equivalent amount of nalbuphine free base, prodrug, non-hydrochloride salt, or mixture thereof.

197. A composition according to claim 193 comprising nalbuphine hydrochloride, the amount of nalbuphine hydrochloride salt being 10 to 15 times greater, by weight, than the amount of naloxone hydrochloride salt, or an equivalent amount of nalbuphine free base, prodrug, non-hydrochloride salt, or mixture thereof.

198. A composition according to claim 193 comprising nalbuphine hydrochloride, the amount of nalbuphine hydrochloride salt being 12.5 times greater, by weight, than the amount of naloxone hydrochloride salt, or an equivalent amount of nalbuphine free base, prodrug, non-hydrochloride salt, or mixture thereof.

199. A composition according to claim 193 comprising 1.25 mg to 10 mg of nalbuphine hydrochloride salt, or an equivalent amount of nalbuphine free base, prodrug, non-hydrochloride salt, or mixture thereof.

200. A composition according to claim 193 comprising 0.1 mg to 0.8 mg of naloxone hydrochloride salt or an equivalent amount of naloxone free base, prodrug, non-hydrochloride salt, or mixture thereof.

201. A composition according to claim 193 comprising 1 mg to 2.5 mg of nalbuphine hydrochloride salt, or an equivalent amount of nalbuphine free base, prodrug, non-chloride salt, or mixture thereof and 0.1 to 0.8 naloxone hydrochloride salt or an equivalent amount of naloxone free base, prodrug, non-hydrochloride salt or mixture thereof.

202. A composition according to claim 193 comprising 8.5 mg to 10 mg of nalbuphine hydrochloride salt, or an equivalent amount of nalbuphine free base, prodrug, non-chloride salt, or mixture thereof and 0.1 to 0.8 naloxone hydrochloride salt or an equivalent amount of naloxone free base, prodrug, non-hydrochloride salt or mixture thereof.

203. A composition according to claim 193 comprising 0.4 mg of naloxone hydrochloride and 5 mg of nalbuphine hydrochloride.

204. A composition according to claim 193 comprising 0.125 mg of naloxone hydrochloride and 2.5 mg of nalbuphine hydrochloride.

205. A composition according to claim 193 comprising 0.2 mg of naloxone hydrochloride and 2.5 mg of nalbuphine hydrochloride.

206. A composition according to claim 193 comprising 0.8 mg of naloxone hydrochloride and 10 mg of nalbuphine hydrochloride.

207. A composition according to claim 193 formulated for intranasal administration.

208. A composition according to claim 193 formulated for pulmonary administration.

209. A composition for treating pain comprising (a) 0.1 mg to 0.8 mg of a hydrochloride salt of an opioid antagonist or an equivalent amount of opioid antagonist free base, prodrug, non-hydrochloride salt, or mixture thereof, wherein said opioid antagonist is naloxone, naltrexone, methylnaltrexone, nalmefene, nalorphine, levalorphan, oxylorphan or cyprenorphine; (b) an amount of a free base, hydrochloride salt, prodrug, non-hydrochloride salt or mixture thereof of a kappa opioid, that results in greater analgesia than administration of either said opioid antagonist hydrochloride salt or said kappa-opioid, wherein the kappa-opioid is pentazocine, butorphanol, ketazocine, ethylketazocine, dezocine, bremazocine, a benzacetamide derivative, a phenothiazine derivative, a thiazine derivative, or a benzodiazepine derivative; and (c) a pharmaceutically acceptable carrier; provided that the composition does not comprise 60 mg of pentazocine free base or an equivalent amount of pentazocine hydrochloride salt, non-hydrochloride salt, prodrug, or mixture thereof and 0.4 mg naloxone free base or equivalent amount of naloxone hydrochloride salt, non-hydrochloride salt, prodrug, or mixture thereof.

210. A composition according to claim 209 wherein the kappa-opioid is pentazocine and the opioid antagonist is naloxone.

211. A composition according to claim 210 comprising pentazocine hydrochloride salt in an amount 18 to 30 times greater, by weight, than the amount of naloxone hydrochloride salt, or an equivalent amount of pentazocine free base, prodrug, non-hydrochloride salt, or mixture thereof.

212. A composition according to claim 209 wherein the kappa-opioid is butorphanol and the opioid antagonist is naloxone.

213. A composition according to claim 212 comprising 0.3 to 10 times greater, by weight, butorphanol tartrate salt than naloxone hydrochloride salt, or equivalent amount of butorphanol free base, non-tartrate salt, prodrug, or mixture thereof and/or naloxone free base, non-hydrochloride salt, prodrug, or mixture thereof.

214. A composition according to claim 212 comprising 0.1 to 0.8 mg naloxone hydrochloride salt or an equivalent amount of naloxone free base, non-hydrochloride salt, prodrug, or mixture thereof.

215. A composition according to claim 209 formulated for intravenous administration.

216. A composition according to claim 209 formulated for mucosal administration.

217. A composition according to claim 209 formulated for intranasal administration.

218. A composition according to claim 209 formulated for pulmonary administration.

219. A composition for treating pain comprising (a) 0.1 to 0.8 mg of a hydrochloride salt of naloxone or an equivalent amount of naloxone free base, prodrug, non-hydrochloride salt, or mixture thereof; (b) 1-2.5 mg of nalbuphine hydrochloride, or an equivalent amount of nalbuphine free base, prodrug, non-hydrochloride salt or mixture thereof; and (c) a pharmaceutically acceptable carrier.

220. A composition for treating pain comprising (a) 0.1 to 0.8 mg of a hydrochloride salt of naloxone or an equivalent amount of naloxone free base, prodrug, non-hydrochloride salt, or mixture thereof; (b) 8.5 to 10 mg of nalbuphine hydrochloride, or an equivalent amount of nalbuphine free base, prodrug, non-hydrochloride salt or mixture thereof; and (c) a pharmaceutically acceptable carrier.

221. A composition for treating pain comprising (a) 0.1 mg of a hydrochloride salt of naloxone or an equivalent amount of naloxone free base, prodrug, non-hydrochloride salt, or mixture thereof; (b) 1.25 mg of nalbuphine hydrochloride, or an equivalent amount of nalbuphine free base, prodrug, non-hydrochloride salt or mixture thereof; and (c) a pharmaceutically acceptable carrier.

222. A composition for treating pain comprising (a) 0.2 mg of a hydrochloride salt of naloxone or an equivalent amount of naloxone free base, prodrug, non-hydrochloride salt, or mixture thereof; (b) 2.5 mg of nalbuphine hydrochloride, or an equivalent amount of nalbuphine free base, prodrug, non-hydrochloride salt or mixture thereof; and (c) a pharmaceutically acceptable carrier.

223. A composition for treating pain comprising (a) 0.8 mg of a hydrochloride salt of naloxone or an equivalent amount of naloxone free base, prodrug, non-hydrochloride salt, or mixture thereof; (b) 10 mg of nalbuphine hydrochloride, or an equivalent amount of nalbuphine free base, prodrug, non-hydrochloride salt or mixture thereof; and (c) a pharmaceutically acceptable carrier.

224. A composition according to claim 193 formulated for treatment of neuropathic pain.

225. A composition according to claim 193 formulated for treatment of inflammatory pain.

226. A composition according to claim 193 formulated for treatment of acute pain.

227. A composition according to claim 193 formulated for treatment of traumatic pain.

228. A composition according to claim 193 formulated for treatment of post-procedural pain.

229. A composition according to claim 193 formulated for treatment of infection-related pain.

230. A method of treating pain comprising mucosally administering to an individual in need of said treatment (a) an amount of an opioid antagonist free base, prodrug, salt, or mixture thereof equivalent to intravenous administration of 0.1 mg to 0.8 mg of a hydrochloride salt of an opioid antagonist, wherein said opioid antagonist is naloxone, naltrexone, methyltrexone, nalmefene, nalorphine, levalorphan, oxylorphan or cyprenorphine, and (b) an amount of nalbuphine free base, hydrochloride salt, prodrug or mixture thereof that results in greater analgesia than administration of either said opioid antagonist free base, prodrug, salt, or mixture thereof or said nalbuphine alone.

231. The method of claim 230 wherein the nalbuphine free base, salt, prodrug, or mixture thereof, and opioid antagonist hydrochloride free base, prodrug, salt, or mixture thereof are administered in amounts equivalent to intravenous administration of 6.25 to 30 times greater in weight of nalbuphine hydrochloride salt than the opioid antagonist hydrochloride salt.

232. The method of claim 230 wherein the nalbuphine free base, salt, prodrug, or mixture thereof, and opioid antagonist hydrochloride free base, prodrug, salt, or mixture thereof are administered in amounts equivalent to intravenous administration of 6.25 to 25 times greater in weight of nalbuphine hydrochloride salt than opioid antagonist hydrochloride salt.

233. The method of claim 230 wherein the nalbuphine free base, salt, prodrug, or mixture thereof, and opioid antagonist hydrochloride free base, prodrug, salt, or mixture thereof are administered in amounts equivalent to intravenous administration of 6.25 to 20 times greater in weight of nalbuphine hydrochloride salt than opioid antagonist hydrochloride salt.

234. The method of claim 230 wherein the nalbuphine free base, salt, prodrug, or mixture thereof, and opioid antagonist hydrochloride free base, prodrug, salt, or mixture thereof are administered in amounts equivalent to intravenous administration of 10 to 20 times greater in weight of nalbuphine hydrochloride salt than opioid antagonist hydrochloride salt.

235. The method of claim 230 wherein the nalbuphine free base, salt, prodrug, or mixture thereof, and opioid antagonist hydrochloride free base, prodrug, salt, or mixture thereof are administered in amounts equivalent to intravenous administration of 5 to 10 times greater in weight of nalbuphine hydrochloride salt than opioid antagonist hydrochloride salt.

236. The method of claim 230 wherein the nalbuphine free base, salt, prodrug, or mixture thereof, is administered in an amount equivalent to intravenous administration of 1 mg to 30 mg of nalbuphine hydrochloride salt.

237. The method of claim 230 wherein the nalbuphine free base, salt, prodrug, or mixture thereof, is administered in an amount equivalent to intravenous administration of 1 mg to 20 mg of nalbuphine hydrochloride salt.

238. The method of claim 230 wherein the nalbuphine free base, salt, prodrug, or mixture thereof, is administered in an amount equivalent to intravenous administration of 1 mg to 10 mg of nalbuphine hydrochloride salt.

239. The method of claim 230 wherein the nalbuphine free base, salt, prodrug, or mixture thereof, is administered in an amount equivalent to intravenous administration of 1 mg to 6 mg of nalbuphine hydrochloride salt.

240. The method of claim 230 wherein the nalbuphine free base, salt, prodrug, or mixture thereof, is administered in an amount equivalent to intravenous administration of 1 mg to 5 mg of nalbuphine hydrochloride salt.

241. The method of any of claims 230-240 wherein the opioid antagonist is naloxone.

242. The method of claim 241 wherein the amount of naloxone free base, salt, or prodrug is equivalent to intravenous administration of 0.1 mg naloxone hydrochloride salt and the amount of nalbuphine free base, salt, or prodrug is equivalent to intravenous administration of 1.25 mg nalbuphine hydrochloride salt.

243. The method of claim 241 wherein the amount of naloxone free base, salt, or prodrug is equivalent to intravenous administration of 0.2 mg naloxone hydrochloride salt and the amount of nalbuphine free base, salt, or prodrug is equivalent to intravenous administration of 2.5 mg nalbuphine hydrochloride salt are administered.

244. The method of claim 241 wherein the amount of naloxone free base, salt, or prodrug is equivalent to intravenous administration of 0.4 mg naloxone hydrochloride salt and the amount of nalbuphine free base, salt, or prodrug is equivalent to intravenous administration of 5 mg nalbuphine hydrochloride salt are administered.

245. The method of claim 241 wherein the amount of naloxone free base, salt, or prodrug is equivalent to intravenous administration of 0.8 mg naloxone hydrochloride salt and the amount of nalbuphine free base, salt, or prodrug is equivalent to intravenous administration of 10 mg nalbuphine hydrochloride salt are administered.

246. The method of claim 230 wherein the method of mucosal administration is intranasally.

247. The method of claim 230 wherein the method of mucosal administration is pulmonary.

248. A method of treating pain comprising mucosally administering to an individual in need of said treatment (a) a first amount of an opioid antagonist as a free base, prodrug, salt, or mixture thereof, wherein said opioid antagonist is naloxone, naltrexone, methyltrexone, nalmefene, nalorphine, levalorphan, oxylorphan or cyprenorphine, and (b) a second amount of nalbuphine as a free base, salt, prodrug or mixture, wherein said first amount and said second amount are equivalent to intravenous administration of a weight ratio of 1 to 12.5 of said opioid antagonist free base and nalbuphine free base and wherein said administration results in greater analgesia than administration of either said opioid antagonist or said nalbuphine alone.

249. The method of claim 248 wherein said second amount is equivalent to intravenous administration of 1 mg to 30 mg of nalbuphine hydrochloride salt.

250. The method of claim 248 wherein said second amount is equivalent to intravenous administration of 1 mg to 20 mg of nalbuphine hydrochloride salt.

251. The method of claim 248 wherein said second amount is equivalent to intravenous administration of 1 mg to 10 mg of nalbuphine hydrochloride salt.

252. The method of claim 248 wherein said second amount is equivalent to intravenous administration of 1 mg to 5 mg of nalbuphine hydrochloride salt.

253. The method of claim 248 wherein the opioid antagonist is naloxone hydrochloride salt or an equivalent amount of free base, non-hydrochloride salt, prodrug, or mixture thereof.

254. The method of claim 253 wherein said first amount is an amount of naloxone free base, salt, or prodrug equivalent to intravenous administration of 0.1 mg naloxone hydrochloride salt and said second amount is equivalent to intravenous administration of 1.25 mg nalbuphine hydrochloride salt.

255. The method of claim 253 wherein said first amount is an amount of naloxone free base, salt, or prodrug equivalent to intravenous administration of 0.2 mg naloxone hydrochloride salt and said second amount is equivalent to intravenous administration of 2.5 mg nalbuphine hydrochloride salt.

256. The method of claim 253 wherein said first amount is an amount of naloxone free base, salt, or prodrug equivalent to intravenous administration of 0.4 mg naloxone hydrochloride salt and said second amount is equivalent to intravenous administration of 5 mg nalbuphine hydrochloride salt.

257. The method of claim 253 wherein said first amount is an amount of naloxone free base, salt, or prodrug equivalent to intravenous administration of 0.8 mg naloxone hydrochloride salt and said second amount is equivalent to intravenous administration of 10 mg nalbuphine hydrochloride salt.

258. The method of claim 253 wherein said first amount is an amount of naloxone free base, salt, or prodrug equivalent to intravenous administration of 1.6 mg naloxone hydrochloride salt and said second amount is equivalent to intravenous administration of 20 mg nalbuphine hydrochloride salt.

259. The method of claim 253 wherein said first amount is an amount of naloxone free base, salt, or prodrug equivalent to intravenous administration of 2.0 mg naloxone hydrochloride salt and said second amount is equivalent to intravenous administration of 30 mg nalbuphine hydrochloride salt.

260. The method of claim 253 wherein the method of mucosal administration is intranasally.

261. The method of claim 253 wherein the method of mucosal administration is pulmonary.

262. A method of treating pain comprising administering to an individual in need of said treatment (a) a first amount of an opioid antagonist as a free base, prodrug, salt, or mixture thereof, wherein said opioid antagonist is naloxone, naltrexone, methyltrexone, nalmefene, nalorphine, levalorphan, oxylorphan or cyprenorphine, and (b) a second amount of nalbuphine as a free base, salt, prodrug or mixture, wherein said first amount and said second amount are equivalent to intravenous administration of a weight ratio of said opioid antagonist free base and said nalbuphine free base that is 1:6.25 to 1:49 and wherein said administration results in greater analgesia than administration of either said opioid antagonist or said nalbuphine alone, provided that 5 mg nalbuphine free base or an equivalent amount of nalbuphine hydrochloride salt, non-hydrochloride salt, prodrug, or mixture thereof, is not intravenously administered with 0.4 mg naloxone free base or equivalent amount of naloxone hydrochloride salt, non-hydrochloride salt, prodrug, or mixture thereof.

263. A method of treating pain comprising administering to an individual in need of said treatment (a) a first amount of an opioid antagonist as a free base, prodrug, salt, or mixture thereof, wherein said opioid antagonist is naloxone, naltrexone, methyltrexone, nalmefene, nalorphine, levalorphan, oxylorphan or cyprenorphine, and (b) a second amount of nalbuphine as a free base, salt, prodrug or mixture, wherein said first amount and said second amount are equivalent to intravenous administration of a weight ratio of said opioid antagonist free base and said nalbuphine free base that is 1:6.25 to 1:7 and wherein said administration results in greater analgesia than administration of either said opioid antagonist or said nalbuphine alone.

264. A method of treating pain comprising administering to an individual in need of said treatment (a) a first amount of an opioid antagonist as a free base, prodrug, salt, or mixture thereof, wherein said opioid antagonist is naloxone, naltrexone, methyltrexone, nalmefene, nalorphine, levalorphan, oxylorphan or cyprenorphine, and (b) a second amount of nalbuphine as a free base, salt, prodrug or mixture, wherein said first amount and said second amount are equivalent to intravenous administration of a weight ratio of said opioid antagonist free base and said nalbuphine free base that is 1:7 to 1:9 and wherein said administration results in greater analgesia than administration of either said opioid antagonist or said nalbuphine alone.

265. A method of treating pain comprising administering to an individual in need of said treatment (a) a first amount of an opioid antagonist as a free base, prodrug, salt, or mixture thereof, wherein said opioid antagonist is naloxone, naltrexone, methyltrexone, nalmefene, nalorphine, levalorphan, oxylorphan or cyprenorphine, and (b) a second amount of nalbuphine as a free base, salt, prodrug or mixture, wherein said first amount and said second amount are equivalent to intravenous administration of a weight ratio of said opioid antagonist free base and said nalbuphine free base that is 1:9 to 1:11 and wherein said administration results in greater analgesia than administration of either said opioid antagonist or said nalbuphine alone.

266. A method of treating pain comprising administering to an individual in need of said treatment (a) a first amount of an opioid antagonist as a free base, prodrug, salt, or mixture thereof, wherein said opioid antagonist is naloxone, naltrexone, methyltrexone, nalmefene, nalorphine, levalorphan, oxylorphan or cyprenorphine, and (b) a second amount of nalbuphine as a free base, salt, prodrug or mixture, wherein said first amount and said second amount are equivalent to intravenous administration of a weight ratio of said opioid antagonist free base and said nalbuphine free base that is 1:11 to 1:13 and wherein said administration results in greater analgesia than administration of either said opioid antagonist or said nalbuphine alone, provided that 5 mg nalbuphine free base or an equivalent amount of nalbuphine hydrochloride salt, non-hydrochloride salt, prodrug, or mixture thereof, is not intravenously administered with 0.4 mg naloxone free base or equivalent amount of naloxone hydrochloride salt, non-hydrochloride salt, prodrug, or mixture thereof.

267. A method of treating pain comprising administering to an individual in need of said treatment (a) a first amount of an opioid antagonist as a free base, prodrug, salt, or mixture thereof, wherein said opioid antagonist is naloxone, naltrexone, methyltrexone, nalmefene, nalorphine, levalorphan, oxylorphan or cyprenorphine, and (b) a second amount of nalbuphine as a free base, salt, prodrug or mixture, wherein said first amount and said second amount are equivalent to intravenous administration of a weight ratio of said opioid antagonist free base and said nalbuphine free base that is 1:13 to 1:15 and wherein said administration results in greater analgesia than administration of either said opioid antagonist or said nalbuphine alone.

268. A method of treating pain comprising administering to an individual in need of said treatment (a) a first amount of an opioid antagonist as a free base, prodrug, salt, or mixture thereof, wherein said opioid antagonist is naloxone, naltrexone, methyltrexone, nalmefene, nalorphine, levalorphan, oxylorphan or cyprenorphine, and (b) a second amount of nalbuphine as a free base, salt, prodrug or mixture, wherein said first amount and said second amount are equivalent to intravenous administration of a weight ratio of said opioid antagonist free base and said nalbuphine free base that is 1:15 to 1:20 and wherein said administration results in greater analgesia than administration of either said opioid antagonist or said nalbuphine alone.

269. A method of treating pain comprising administering to an individual in need of said treatment (a) a first amount of an opioid antagonist as a free base, prodrug, salt, or mixture thereof, wherein said opioid antagonist is naloxone, naltrexone, methyltrexone, nalmefene, nalorphine, levalorphan, oxylorphan or cyprenorphine, and (b) a second amount of nalbuphine as a free base, salt, prodrug or mixture, wherein said first amount and said second amount are equivalent to intravenous administration of a weight ratio of said opioid antagonist free base and said nalbuphine free base that is 1:20 to 1:30 and wherein said administration results in greater analgesia than administration of either said opioid antagonist or said nalbuphine alone.

270. A method of treating pain comprising administering to an individual in need of said treatment (a) a first amount of an opioid antagonist as a free base, prodrug, salt, or mixture thereof, wherein said opioid antagonist is naloxone, naltrexone, methyltrexone, nalmefene, nalorphine, levalorphan, oxylorphan or cyprenorphine, and (b) a second amount of nalbuphine as a free base, salt, prodrug or mixture, wherein said first amount and said second amount are equivalent to intravenous administration of a weight ratio of said opioid antagonist free base and said nalbuphine free base that is 1:30 to 1:49 and wherein said administration results in greater analgesia than administration of either said opioid antagonist or said nalbuphine alone.

271. The method of claim 262, wherein the second amount of nalbuphine hydrochloride salt is 1 mg to 5 mg, or an equivalent amount of nalbuphine free base, prodrug, non-hydrochloride salt, or a mixture of one or more thereof.

272. The method of claim 262, wherein the second amount of nalbuphine hydrochloride salt is 1 mg to 10 mg, or an equivalent amount of nalbuphine free base, prodrug, non-hydrochloride salt, or a mixture of one or more thereof.

273. The method of claim 262, wherein the second amount of nalbuphine hydrochloride salt is 1 mg to 20 mg, or an equivalent amount of nalbuphine free base, prodrug, non-hydrochloride salt, or a mixture of one or more thereof.

274. The method of claim 262, wherein the second amount of nalbuphine hydrochloride salt is 1 mg to 30 mg, or an equivalent amount of nalbuphine free base, prodrug, non-hydrochloride salt, or a mixture of one or more thereof.

275. The method of claim 262, wherein the method of administration is intranasal.

276. The method of claim 262, wherein the method of administration is intravenous.

277. The method of claim 262, wherein the method of administration is pulmonary.

278. The method of claim 262, wherein the method of administration is transdermal.

279. The method of claim 262, wherein the method of administration is mucosal.

280. A method of treating pain comprising mucosally administering to an individual in need of said treatment (a) 0.02 mg to 8 mg of a hydrochloride salt of an opioid antagonist or an equivalent amount of opioid antagonist free base, prodrug, non-hydrochloride salt, or mixture thereof, wherein said opioid antagonist is naloxone, naltrexone, methylnaltrexone, nalmefene, nalorphine, levalorphan, oxylorphan or cyprenorphine, and (b) an amount of nalbuphine free base, hydrochloride salt, prodrug, non-hydrochloride salt or mixture thereof that results in greater analgesia than administration of either said opioid antagonist hydrochloride salt or said nalbuphine alone.

281. A pharmaceutical composition comprising (a) 0.02 mg to 8 mg of a hydrochloride salt of an opioid antagonist or an equivalent amount of opioid antagonist free base, prodrug, non-hydrochloride salt, or mixture thereof, wherein said opioid antagonist is naloxone, naltrexone, methylnaltrexone, nalmefene, nalorphine, levalorphan, oxylorphan or cyprenorphine; (b) an amount of nalbuphine free base, nalbuphine salt, nalbuphine prodrug, or mixture thereof that results in greater analgesia than administration of the nalbuphine ingredient or the opioid antagonist ingredient alone; and (c) a pharmaceutically acceptable carrier, said pharmaceutical composition being formulated for mucosal administration.

282. A method of treating pain comprising mucosally administering to an individual in need of said treatment (a) an amount of an opioid antagonist free base, prodrug, salt, or mixture thereof equivalent to intravenous administration of 0.02 mg to 8 mg of a hydrochloride salt of an opioid antagonist, wherein said opioid antagonist is naloxone, naltrexone, methyltrexone, nalmefene, nalorphine, levalorphan, oxylorphan or cyprenorphine, and (b) an amount of nalbuphine free base, hydrochloride salt, prodrug or mixture thereof that results in greater analgesia than administration of either said opioid antagonist free base, prodrug, salt, or mixture thereof or said nalbuphine alone.

283. A method of treating pain comprising administering to an individual in need of said treatment (a) from 0.02 to 8 mg of a hydrochloride salt of naloxone or an equivalent amount of naloxone free base, prodrug, non-hydrochloride salt, or mixture thereof; and (b) from 1 to 2.5 mg of nalbuphine hydrochloride, or an equivalent amount of nalbuphine free base, prodrug, non-hydrochloride salt or mixture thereof.

284. A method of treating pain comprising administering to an individual in need of said treatment (a) from 0.02 to 8 mg of a hydrochloride salt of naloxone or an equivalent amount of naloxone free base, prodrug, non-hydrochloride salt, or mixture thereof; and (b) from 8.5 to 30 mg of nalbuphine hydrochloride, or an equivalent amount of nalbuphine free base, prodrug, non-hydrochloride salt or mixture thereof.

285. A pharmaceutical composition for treating pain comprising (a) 0.02 mg to 8 mg of a hydrochloride salt of an opioid antagonist or an equivalent amount of opioid antagonist free base, prodrug, non-hydrochloride salt, or mixture thereof, wherein said opioid antagonist is naloxone, naltrexone, methylnaltrexone, nalmefene, nalorphine, levalorphan, oxylorphan or cyprenorphine, (b) an amount of nalbuphine free base, hydrochloride salt, prodrug, non-hydrochloride salt or mixture thereof that results in greater analgesia than administration of either said opioid antagonist hydrochloride salt or said nalbuphine alone; and (c) a pharmaceutically acceptable carrier; provided that said composition formulated for intravenous administration does not comprise 5 mg nalbuphine free base with 0.4 mg naloxone free base.

286. A composition for treating pain comprising (a) 0.02 mg to 8 mg of a hydrochloride salt of an opioid antagonist or an equivalent amount of opioid antagonist free base, prodrug, non-hydrochloride salt, or mixture thereof, wherein said opioid antagonist is naloxone, naltrexone, methylnaltrexone, nalmefene, nalorphine, levalorphan, oxylorphan or cyprenorphine; (b) an amount of a free base, hydrochloride salt, prodrug, non-hydrochloride salt or mixture thereof of a kappa opioid, that results in greater analgesia than administration of either said opioid antagonist hydrochloride salt or said kappa-opioid, wherein the kappa-opioid is pentazocine, butorphanol, ketazocine, ethylketazocine, dezocine, bremazocine, a benzacetamide derivative, a phenothiazine derivative, a thiazine derivative, or a benzodiazepine derivative; and (c) a pharmaceutically acceptable carrier; provided that said composition formulated for intravenous administration does not comprise 60 mg of pentazocine free base or an equivalent amount of pentazocine hydrochloride salt, non-hydrochloride salt, prodrug, or mixture thereof and 0.4 mg naloxone free base or equivalent amount of naloxone hydrochloride salt, non-hydrochloride salt, prodrug, or mixture thereof and provided that said composition formulated for oral administration does not comprise 50 mg of pentazocine free base or an equivalent amount of pentazocine hydrochloride salt, non-hydrochloride salt, prodrug, or mixture thereof and 0.5 mg naloxone free base or equivalent amount of naloxone hydrochloride salt, non-hydrochloride salt, prodrug, or mixture thereof.
